(19) United States
(12) Patent Application Publication
(10) Pub. No.: US 2010/0285047 A1
(43) Pub. Date: Nov. 11, 2010

(54) THERAPEUTICS AND DIAGNOSTICS FOR GROUP A STREPTOCOCCI

(76) Inventors: Kowthar Salim, London (CA); Joyce De Azavedo, Toronto (CA); Dennis Cvitkovitch, Oakville (CA)

Correspondence Address: HOWSON & HOWSON LLP, 501 OFFICE CENTER DRIVE, SUITE 210, FORT WASHINGTON, PA, 19034, US

(21) Appl. No.: 12/667,107

(22) PCT Filed: Jul. 3, 2008
(86) PCT No.: PCT/CA2008/001246

§ 371 Date: Jul. 19, 2010

Related U.S. Application Data
(60) Provisional application No. 60/958,218, filed on Jul. 3, 2007.
(60) Provisional application No. 60/999,307, filed on Oct. 17, 2007.

(51) Int. Cl. A61K 39/09; G01N 33/50; G01N 33/53; C12Q 1/68; C07K 16/12; A61P 31/04; A61P 37/04

(52) U.S. Cl. 424/190.1; 424/244.1; 436/86; 436/501; 436/94; 435/6; 530/389.5

(57) ABSTRACT

Immunogenic compositions and vaccines are described comprising GAS Markers. Methods for detecting GAS diseases in a subject are also described comprising measuring GAS markers in a sample from the subject. The invention further provides kits for carrying out the methods of the invention and therapeutic applications for GAS diseases employing GAS markers, polynucleotides encoding the markers, and/or binding agents for the markers.

### FIELD OF THE INVENTION

The invention relates to compositions and methods for the diagnosis, treatment, prevention and amelioration of diseases caused by Group A Streptococcus.

### BACKGROUND OF THE INVENTION

Group A Streptococcus (GAS), also known as Streptococcus pyogenes, cause several types of disease in humans, including strep throat, scarlet fever, impetigo, cellulitis-erysipelas, rheumatic fever, acute glomerular nephritis, endocarditis, and necrotizing fasciitis and it is associated with significant morbidity and mortality worldwide (Carapetis, J. R., Steer, A. C. et al). The development of effective and safe vaccines against streptococcal infections has been ongoing (Bisno, A. L., Rubin, F. A. et al). A useful vaccine against GAS would reduce health care costs and numerous physician visits.

A number of group A Streptococcus vaccine candidates have been identified, such as M proteins (Bessen, D. et al; Fischetti, V. A. 1989 Infect. Immun. 64:1495-1501; Lancefield, R. C. 1962, J. Immun. 89:307-313), C5a peptidase (Cleary, P. P., Matsuka, Y. V. et al; Kapur, V. et al. 1994 Infect Immun. 65:2080-2087), cysteine protease (Dale, J. B., et al, Microb. Pathogenesis. 16:443-450) and lipoteichoic acid (Dale, J. B., et al., 1996 J. Infect. Dis. 169:319-323; Lancefield, R. C. 1962; Clin. Microbiol. 2:285-314). However, there are difficulties associated with a vaccine strategy involving the M protein, such as the large number of serologic M types, and the observation that some M proteins contain epitopes that cross-react with human tissues. Thus, a need still exists for a flexible, effective, and multivalent vaccine against GAS.

### SUMMARY OF THE INVENTION

The invention provides markers and marker sets that distinguish Group A Streptococcus diseases (GAS diseases). A marker set may comprise or consist of a plurality of GAS polypeptides and/or polynucleotides selected from the polynucleotide and polypeptide markers set out in Tables 3, 4 and 5 (hereinafter “GAS markers”). GAS markers and marker sets can be used for diagnosis, monitoring (i.e. monitoring progression or therapeutic treatment), prognosis, treatment, or classification of a GAS disease. While the GAS markers are presented together in a group in Tables 3, 4 and 5, each of the sequences can be separately considered and claimed.

An aspect of the invention provides a composition of matter comprising a purified polypeptide consisting essentially of one or more of the polypeptides in Tables 3, 4 and 5, or a fragment thereof. The purified polypeptide may further comprise a carrier or be linked to an indicator reagent (e.g. detectable substance), an amino acid spacer, an amino acid linker, a signal sequence, a stop transfer sequence, a transmembrane domain, a protein purification ligand or a combination thereof.

The levels of markers or marker sets in a sample may be determined by methods as described herein and generally known in the art.

In an aspect, the invention provides a method for characterizing or classifying a patient sample comprising detecting a difference in the expression of a first plurality of GAS markers relative to a control, the first plurality of GAS markers consisting of one or more markers set out in Tables 3, 4 and 5.

In an embodiment of the invention, a method is provided for diagnosing a GAS disease in a patient comprising:

(a) obtaining a sample from a patient;

(b) detecting in the sample at least one GAS marker; and

(c) comparing the detected amount with an amount detected for a standard.

The term “detect” or “detecting” includes assaying or otherwise establishing the presence or absence of the target markers, subunits thereof, or combinations of reagent bound targets, and the like, or assaying for, ascertaining, establishing, or otherwise determining one or more factual characteristics of a GAS disease. The term encompasses diagnostic, prognostic, and monitoring applications for the markers.

The invention also provides a method of assessing whether a patient is afflicted with or has a pre-disposition for a GAS disease the method comprising comparing:
(a) levels of GAS polypeptide or polynucleotide markers associated with a GAS disease in a sample from the patient; and
(b) normal levels of GAS markers in samples of the same type obtained from control patients not afflicted with the disease, wherein altered levels of the markers relative to the corresponding normal levels of markers is an indication that the patient is afflicted with a GAS disease.

In an aspect of a method of the invention for assessing whether a patient is afflicted with or has a pre-disposition for a GAS disease, higher levels of the markers in a sample relative to the corresponding normal levels is an indication that the patient is afflicted with or has a pre-disposition for a GAS disease.

In another aspect of a method of the invention for assessing whether a patient is afflicted with or has a pre-disposition for a GAS disease, lower levels of GAS markers in a sample relative to the corresponding normal levels is an indication that the patient is afflicted with a GAS disease.

In a further aspect, a method for screening a subject for a GAS disease is provided comprising (a) obtaining a biological sample from a subject; (b) detecting the amount of GAS markers in said sample; and (c) comparing said amount of markers detected to a predetermined standard, where detection of a level of markers that differs significantly from the standard indicates a GAS disease.

In an embodiment, a significant difference between the levels of GAS marker levels in a patient and normal levels is an indication that the patient is afflicted with or has a predisposition to a GAS disease.

In a particular embodiment the amount of GAS marker(s) detected is greater than that of a standard and is indicative of a GAS disease. In another particular embodiment, the amount of GAS marker(s) detected is lower than that of a standard and is indicative of a GAS disease.

In particular, the invention provides a non-invasive method for detection, diagnosis or prediction of a GAS disease in a subject comprising: obtaining a sample of blood, plasma, serum, urine or saliva or a tissue sample from the subject; subjecting the sample to a procedure to detect GAS markers in the blood, plasma, serum, urine, saliva or tissue; detecting, diagnosing, and predicting GAS disease by comparing the levels of GAS markers to the levels of marker(s) or polynucleotide(s) obtained from a control subject with no GAS disease.

In aspect, the invention provides a method for monitoring the progression of a GAS disease in a patient the method comprising:
(a) detecting GAS markers in a sample from the patient at a first time point;
(b) repeating step (a) at a subsequent point in time; and
(c) comparing the levels detected in (a) and (b), and therefrom monitoring the progression of the GAS disease.

The invention contemplates a method for determining the effect of an environmental factor on a GAS disease comprising comparing GAS markers in the presence and absence of the environmental factor.

The invention further relates to a method of assessing the efficacy of a therapy for inhibiting a GAS disease in a patient. A method of the invention comprises comparing: (a) levels of GAS markers in a first sample from the patient obtained from the patient prior to providing at least a portion of the therapy to the patient; and (b) levels of GAS markers in a second sample obtained from the patient following therapy.

In an embodiment, a significant difference between the levels of GAS markers in the second sample relative to the first sample is an indication that the therapy is efficacious for inhibiting GAS disease. In a particular embodiment, the method is used to assess the efficacy of a therapy for inhibiting GAS disease, where lower levels of GAS markers in the second sample relative to the first sample, is an indication that the therapy is efficacious for inhibiting the disease. The “therapy” may be any therapy for treating GAS disease, including but not limited to antibiotics. Therefore, the method can be used to evaluate a patient before, during, and after therapy.

Certain methods of the invention employ binding agents (e.g. antibodies) that specifically recognize GAS markers. In an embodiment, the invention provides methods for determining the presence or absence of GAS disease in a patient, comprising the steps of (a) contacting a biological sample obtained from a patient with one or more binding agent that specifically binds to one or more GAS markers; and (b) detecting in the sample an amount of marker that binds to the binding agent, relative to a predetermined standard or cut-off value, and therefrom determining the presence or absence of GAS disease in the patient.

In another embodiment, the invention relates to a method for diagnosing and monitoring a GAS disease in a subject by quantitating one or more GAS markers associated with the disease in a biological sample from the subject comprising (a) reacting the biological sample with one or more binding agent specific for the GAS markers (e.g. an antibody) that are directly or indirectly labelled with a detectable substance; and (b) detecting the detectable substance.

In another aspect the invention provides a method for using an antibody to detect expression of one or more GAS marker in a sample, the method comprising: (a) combining antibodies specific for one or more GAS marker with a sample under conditions which allow the formation of antibody marker complexes; and (b) detecting complex formation, wherein complex formation indicates expression of the marker in the sample. Expression may be compared with standards and is diagnostic of a GAS disease.

Embodiments of the methods of the invention involve (a) reacting a biological sample from a subject with antibodies specific for one or more GAS markers which are directly or indirectly labelled with an enzyme; (b) adding a substrate for the enzyme wherein the substrate is selected so that the substrate, or a reaction product of the enzyme and substrate forms fluorescent complexes; (c) quantitating one or more GAS markers in the sample by measuring fluorescence of the fluorescent complexes; and (d) comparing the quantitated levels to levels obtained for other samples from the subject patient, or control subjects.

In another embodiment the quantitated levels are compared to levels quantitated for control subjects without a GAS disease (e.g. uninfected individuals) wherein an increase in GAS marker levels compared with the control subjects is indicative of GAS disease.

A particular embodiment of the invention comprises the following steps
(a) incubating a biological sample with first antibodies specific for one or more GAS markers which are directly or indirectly labeled with a detectable substance, and second antibodies specific for one or more GAS markers which are immobilized;
(b) detecting the detectable substance thereby quantitating GAS markers in the biological sample; and
(c) comparing the quantitated GAS markers with levels for a predetermined standard.

The standard may correspond to levels quantitated for samples from control subjects without a GAS disease (uninfected individuals) or from other samples of the subject. In an embodiment, increased levels of GAS markers as compared to the standard may be indicative of a GAS disease.

GAS marker levels can be determined by constructing an antibody microarray in which binding sites comprise immobilized antibodies (preferably monoclonal antibodies) specific to a substantial fraction of marker-derived GAS marker polypeptides of interest.

Other methods of the invention employ one or more polynucleotides capable of hybridizing to one or more polynucleotides encoding GAS markers. Thus, methods for detecting GAS markers can be used to monitor a GAS disease by detecting polynucleotide markers associated with the disease. Thus, the present invention relates to a method for diagnosing and monitoring a GAS disease in a sample from a subject comprising isolating nucleic acids, preferably mRNA, from the sample; and detecting GAS marker polynucleotides associated with the disease in the sample. The presence of different levels of GAS marker polynucleotides in the sample compared to a standard or control may be indicative of disease, disease stage, and/or a positive prognosis i.e. longer progression-free and overall survival.

The invention provides methods for determining the presence or absence of a GAS disease in a subject comprising detecting in the sample levels of nucleic acids that hybridize to one or more GAS marker polynucleotides, comparing the levels with a predetermined standard or cut-off value, and therefrom determining the presence or absence of GAS disease in the subject. In an embodiment, the invention provides methods for determining the presence or absence of a GAS disease in a subject comprising (a) contacting a sample obtained from the subject with oligonucleotides that hybridize to one or more GAS marker polynucleotides; and (b) detecting in the sample a level of nucleic acids that hybridize to the polynucleotides relative to a predetermined cut-off value, and therefrom determining the presence or absence of GAS disease in the subject.

Within certain embodiments, the amount of polynucleotides that are mRNA are detected via polymerase chain reaction using, for example, oligonucleotide primers that hybridize to one or more GAS marker polynucleotides, or complements of such polynucleotides. Within other embodiments, the amount of mRNA is detected using a hybridization technique, employing oligonucleotide probes that hybridize to one or more GAS marker polynucleotides, or complements thereof.

When using mRNA detection, the method may be carried out by combining isolated mRNA with reagents to convert to cDNA according to standard methods; treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of one or more GAS polynucleotide markers in the sample. For mRNA the analyzing step may be accomplished using Northern Blot analysis to detect the presence of GAS markers. The analysis step may be further accomplished by quantitatively detecting the presence of GAS markers in the amplification product, and comparing the quantity of markers detected against a panel of expected values for the known presence of the markers in samples from uninfected individuals derived using similar primers.

Therefore, the invention provides a method wherein mRNA is detected by (a) isolating mRNA from a sample and combining the mRNA with reagents to convert it to cDNA; (b) treating the converted cDNA with amplification reaction reagents and nucleic acid primers that hybridize to one or more GAS marker polynucleotides to produce amplification products; (d) analyzing the amplification products to detect an amount of mRNA encoding the GAS markers; and (e) comparing the amount of mRNA to an amount detected against a panel of expected values for normal samples (derived using similar nucleic acid primers).

In particular embodiments of the invention, the methods described herein utilize the GAS marker polynucleotides placed on a microarray so that the expression status of each of the markers is assessed simultaneously.

In a particular aspect, the invention provides a microarray comprising a defined set of genes (e.g., at least 5, 10, 15 or 20 of the genes in Tables 3, 4 and 5). The invention further relates to the use of the microarray as a prognostic tool to predict a GAS disease.

In an embodiment, the invention provides for oligonucleotide arrays comprising GAS marker sets described herein. The microarrays provided by the present invention may comprise probes to markers able to distinguish a GAS disease. In particular, the invention provides oligonucleotide arrays comprising probes to a subset or subsets of gene markers up to a full set of markers which distinguish GAS disease.

The invention provides a method of detecting antibodies that specifically bind GAS or a GAS marker polypeptide. The method can comprise reacting one or more GAS marker polypeptide, in particular a polypeptide in Tables 3, 4 and 5 with a test sample suspected of comprising antibodies specific for a GAS marker polypeptide under conditions that allow polypeptide/antibody complexes to form and detecting polypeptide/antibody complexes. The detection of polypeptide/antibody complexes is an indication that antibodies specific for a GAS marker polypeptide are present in the test sample, and the absence of the polypeptide/antibody complexes is an indication that antibodies specific for GAS marker polypeptides are not present in the test sample. The antibodies can be fragments of antibodies. In aspects of this method of the invention, the amount of antibodies in the test sample can be determined. In aspects of this method of the invention the polypeptide can be attached to a carrier or support. In aspects of this method of the invention the polypeptide can be attached to a detectable substance. In aspects of this method of the invention the polypeptide/antibody complexes can be detected using a labeled anti-species antibody. The method can comprise an assay selected from the group consisting of a microtiter plate assay, a reversible flow chromatographic binding assay, a lateral flow immunoassay, an enzyme linked immunosorbent assay, a radioimmunoassay, a hemaglutination assay, a western blot assay, a fluorescence polarization immunoassay and an indirect immunofluorescence assay.

The invention provides a method of detecting a GAS disease or infection in a subject. The method comprises obtaining a sample from the subject, contacting one or more GAS marker polypeptide or purified GAS marker polypeptide with the sample under conditions that allow polypeptide/antibody complexes to form; and detecting polypeptide/antibody complexes. The detection of polypeptide/antibody complexes is an indication that the mammal has a GAS disease and the absence of polypeptide/antibody complexes is an indication that the mammal does not have a GAS disease. In a method of the invention for detecting antibodies specific for GAS marker polypeptides, the GAS marker polypeptides or antigens comprise one or more epitopes (i.e., antigenic determinants).

In an aspect, the invention provides a method of detecting presence or absence of an antibody specific for a GAS marker polypeptide in a test sample comprising: contacting a test sample with a purified immunogenic GAS marker polypeptide, wherein the polypeptide specifically binds an antibody specific for a GAS marker polypeptide under conditions that allow formation of an immunocomplex between the antibody and the polypeptide; and detecting an immunocomplex, wherein detection of the immunocomplex indicates the presence of antibody specific for a GAS marker polypeptide in the test sample.

The invention also relates to kits for carrying out the methods of the invention, in particular diagnostic methods of the invention. In an embodiment, a kit is for assessing whether a patient is afflicted with a GAS disease and it comprises reagents for assessing one or more GAS markers or antibodies specific for GAS markers. The invention further provides kits comprising marker sets described herein. In an aspect the kit contains a microarray ready for hybridization to target GAS markers, plus software for the data analyses.

The invention also provides a diagnostic composition comprising one or more GAS marker. A composition is also provided comprising a probe that specifically hybridizes to a GAS marker or a fragment thereof, or an antibody specific for GAS markers or a fragment thereof. In another aspect, a composition is provided comprising one or more GAS marker polynucleotide specific primer pairs capable of amplifying the polynucleotides using polymerase chain reaction methodologies. The probes, primers or antibodies can be labeled with a detectable substance.

The invention provides an immunogenic composition for protecting mammals, in particular humans, against infection by Group A Streptococcus. An immunogenic composition of the invention comprises an immunogenic amount of a region of a GAS marker. In a composition of the invention, the region of a GAS marker defines an epitope which induces the formation of bactericidal antibodies against GAS. In an aspect, an immunogenic composition is provided for protecting mammals against infection by Group A Streptococcus comprising an effective amount of a region of at least one Group A Streptococcus marker listed in Tables 3, 4 and 5 that defines an epitope which induces the formation of bactericidal antibodies against GAS. In aspects of the invention the region of the GAS marker is immunoreactive and found in the most prevalent GAS serotypes associated with a selected disease.

The region of a GAS marker present in the immunogenic compositions of the invention may be in the form of a polypeptide or part of a polypeptide (e.g. an epitope). Thus, in an aspect of the invention the immunogenic composition comprises a polypeptide encoded by at least one GAS marker in Tables 3, 4 and 5, or a portion, isoform, homolog, variant, or precursor of the polypeptide, including modified forms of the polypeptide and derivative. An immunogenic protein may also be a chimeric or fusion polypeptide or conjugate.

In embodiments of the invention an immunogenic composition comprises synthetic peptides about 5 to 200, 10 to 150, 10 to 100, 20 to 100, 10 to 50 or 20 to 25 amino acids in length which are portions of one or more GAS marker. In embodiments, the synthetic peptides are serotype specific peptides. In embodiments, the synthetic peptides comprise an epitope of a GAS marker. Synthetic peptides may be used, for example, individually, in a mixture, or in a polypeptide or protein. For example, a polypeptide or protein can be created by fusing or linking the peptides to each other, synthesizing the polypeptide or protein based on the peptide sequences, and linking or fusing the peptides to a backbone.

Immunogenic compositions of the invention are preferably recognized by GAS marker specific antibodies and are capable of eliciting functional opsonic antibodies and/or anti-attachment antibodies without eliciting tissue cross-reactive antibodies.

Immunogenic compositions of the invention may be useful for raising antibodies which have application for prophylactic and diagnostic purposes. Therefore, the invention also provides isolated antibodies that specifically bind to a GAS marker, and in particular antibodies elicited in response to an immunogenic composition or vaccine of the invention. An antibody may be a monoclonal or polyclonal antibody or an antibody fragment (e.g., Fab or F(ab′)₂ fragment). In an aspect, the invention provides antibodies specific for a GAS marker that can be used therapeutically to destroy or inhibit a GAS disease or to block a GAS marker associated with a GAS disease. In an aspect, GAS markers may be used in various immunotherapeutic methods to promote immune-mediated destruction or inhibition of GAS expressing GAS markers. In an aspect, the invention relates to compositions comprising antibodies specific for one or more GAS markers, peptides derived therefrom, or chemically produced (synthetic) peptides, and a pharmaceutically acceptable carrier, excipient, or diluent.

An immunogenic composition of the invention may be useful as a vaccine and the invention contemplates a vaccine comprising an immunogenic composition of the invention.

In an aspect, the invention contemplates vaccines for stimulating or enhancing in a subject to whom the vaccine is administered production of antibodies directed against GAS markers, peptides derived therefrom, or chemically produced (synthetic) peptides, or any combination of these molecules.

An immunogenic composition of this invention may be capable of eliciting active and passive protection against infection by Group A Streptococcus. For passive protection, immunogenic antibodies can be produced by immunizing a human with a vaccine comprising an immunogenic composition of the invention and then recovering the immunogenic antibodies from the human. Thus, the invention contemplates a composition for passive immunization comprising antibodies specific for GAS markers.

In aspects of the invention, an immunogenic composition or vaccine of the invention may be used to inhibit or reduce the growth of Group A Streptococcal bacteria, in particular, S. pyogenes, in blood and/or reduce phagocytic resistance. Accordingly, the invention contemplates the use of GAS markers, peptides derived therefrom, or chemically produced (synthetic) peptides, or any combination of these molecules, for use as vaccines or in the preparation of vaccines to prevent a GAS disease and/or to treat a GAS disease.

An immunogenic composition or vaccine may further comprise additional components, including but not limited to, carriers, diluents, excipients, vehicles (e.g., encapsulated, liposomes), and other immune-stimulatory molecules (e.g., adjuvants, other vaccines). In an aspect, a vaccine further comprises an adjuvant such as aluminum hydroxide, aluminum phosphate, monophosphoryl lipid A, QS21 or stearyl tyrosine.

A polypeptide in an immunogenic composition or vaccine may be conjugated to a native or recombinant bacterial protein such as tetanus toxoid, cholera toxin, diphtheria toxoid, or CRM₁₉₇.

In an aspect, the invention provides methods of immunizing a mammal against infection by Group A Streptococcus by administering an immunogenic amount of a composition of the invention. In an aspect, an immunogenic composition of the invention is used to provide protection against infection by Group A Streptococcus in those populations most at risk of contracting GAS infections and disease namely adults, pregnant women and, in particular, infants and children.

A method for treating or preventing a GAS disease in a patient is also provided comprising administering to a patient in need thereof antibodies specific for one or more GAS markers associated with a GAS disease. The method comprises administering to the subject a vaccine of the invention in a dose effective for stimulating or enhancing production of the antibodies.

The invention further provides a method for treating, preventing, or delaying recurrence of a GAS disease. The method comprises administering to the subject a composition or vaccine of the invention in a dose effective for treating, preventing, or delaying recurrence of a GAS disease.

In further aspects, the invention also relates to methods for using the immunogenic compositions, vaccines, or antibodies and methods for tailoring vaccines. In aspects, the invention also relates to methods for using the immunogenic compositions, vaccines, or antibodies for treating a GAS disease or in the preparation of a medicament for treating a GAS disease.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### Glossary

In accordance with the present invention there may be employed conventional biochemistry, enzymology, molecular biology, microbiology, and recombinant DNA techniques within the skill of the art. Such techniques are explained fully in the literature. See for example, Sambrook et al, Molecular Cloning: A Laboratory Manual, Third Edition (2001) Cold Spring Harbor Laboratory Press, Cold Spring Harbor, N.Y); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

Unless defined otherwise, all technical and scientific terms used herein have the same meaning as commonly understood by one of ordinary skill in the art to which this invention belongs.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”. The term “about” means plus or minus 0.1 to 50%, 5-50%, or 10-40%, preferably 10-20%, more preferably 10% or 15%, of the number to which reference is being made.

The term “sample” means a material known or suspected of expressing or containing one or more GAS markers. A test sample can be used directly as obtained from the source or following a pretreatment to modify the character of the sample. The sample can be a biological sample derived from any biological source, such as tissues, extracts, or cell cultures, including cells, cell lysates, and physiological fluids, such as, for example, blood, plasma, serum, saliva, sputum, ocular lens fluid, cerebrospinal fluid, sweat, urine, feces, amniotic fluid, milk, ascites fluid, synovial fluid, peritoneal fluid, lavage fluid, wound exudates, and the like. The sample can be obtained from animals, preferably mammals, most preferably humans. A sample can also be an environmental sample or a laboratory sample. A sample can be treated prior to use, such as preparing plasma from blood, diluting viscous fluids, and the like. Methods of treatment can involve filtration, distillation, extraction, concentration, inactivation of interfering components, the addition of reagents, and the like. Depending upon the type of test sample, it can be diluted with a suitable buffer reagent, concentrated, or contacted with a solid phase without any manipulation. For example, prior to testing serum or plasma samples can be diluted, or specimens such as urine can be concentrated. In an embodiment the sample is a human physiological fluid. In a particular embodiment, the sample is human serum, urine or plasma.

The terms “subject”, “individual” or “patient” refer to a warm-blooded animal such as a mammal. In particular, the terms refer to a human. The term also includes domestic animals bred for food or as pets, including horses, cows, sheep, poultry, fish, pigs, cats, dogs, and zoo animals. A subject, individual or patient may be afflicted with or suspected of having or being pre-disposed to a GAS disease or at risk of developing a GAS disease. A subject suspected of suffering from a GAS disease or an infection by a GAS displays one or more symptoms of a GAS disease or a GAS infection, or may have come into contact with a person suffering from a GAS disease. A subject at risk of developing a GAS disease is a subject that is exposed to a condition or suffers from a condition that increases the risk of developing a GAS disease or being infected with a GAS.

Methods herein for administering an agent or composition to subjects/individuals/patients contemplate treatment as well as prophylactic use. Typical subjects for treatment or diagnosis include persons susceptible to, suffering from or that have suffered a GAS disease.

The terms “peptide”, “polypeptide” and “protein” are used interchangeably and as used herein refer to more than one amino acid joined by a peptide bond.

“Optional” or “optionally” means that the subsequently described element, event or circumstance may or may not occur, and that the description includes instances where said element, event, or circumstance occurs and instances where it does not.

The term “effective amount” or “effective dose” refers to a non-toxic but sufficient amount of an agent (e.g. antibody) to provide the desired biological effect. The exact amount required will vary from subject to subject, depending on the species, age and general condition of the subject, the particular agent used, its mode of administration, and the like. An appropriate effective amount or effective dose may be determined by one of ordinary skill in the art using routine experimentation.

“Pharmaceutically acceptable” refers to a material that is not biologically or otherwise undesirable, i.e., the material may be administered to an individual without causing any undesirable biological effects or interacting in a deleterious manner with any of the other components of a composition in which it is contained.

“Synthetic” refers to items, e.g., peptides, which are not naturally occurring, in that they are isolated, synthesized or otherwise manipulated by man.

“Immunogenic” as used herein encompasses materials which are capable of producing an immune response.

“Composition” includes any composition of matter, including peptides, polypeptides, proteins, mixtures, vaccines, antibodies, or markers of the present invention.

A “GAS disease” means a disease associated with a Group A Streptococcus, including without limitation streptococcal sore throat (strep throat, pharyngitis), streptococcal skin infections (impetigo, cellulitis, erysipelas), cellulitis and arthritis, peritonitis, scarlet fever, rheumatic fever, postpartum fever, wound infections, pneumonia, invasive group A strep infection, acute glomerulonephritis, necrotizing fasciitis and streptococcal toxic shock syndrome. In aspects of the invention the GAS disease is associated with a clinical strain listed in Table 1. In particular aspects, the GAS disease is a disease listed in Table 2.

A “GAS marker” includes a polypeptide associated with GAS described herein (GAS marker polypeptide”), namely the polypeptides listed in Tables 3, 4 and 5. A “GAS Marker” also includes a polynucleotide associated with GAS described herein (“GAS marker polynucleotide”), namely polynucleotides listed in Tables 3, 4 and 5.

A Gas marker polypeptide includes the native-sequence polypeptide, isoforms, chimeric polypeptides, all homologs, fragments, precursors, complexes, and modified forms and derivatives thereof. A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” includes a polypeptide having at least about 45%, 50%, 55%, 60%, 65%, 70%, 75%, 80%, 85%, 90%, 95%, 97%, 98%, or 99% amino acid sequence identity, particularly at least about 70-80%, more particularly at least about 85%, still more particularly at least about 90%, most particularly at least about 95% amino acid sequence identity with a native-sequence polypeptide. Variants include, for instance, polypeptides wherein one or more amino acid residues are added to, or deleted from, the N- or C-terminus of the full-length or mature sequences of the polypeptide, including variants from other species, but excludes a native-sequence polypeptide. In aspects of the invention variants retain the immunogenic activity of the corresponding native-sequence polypeptide.

Percent identity of two amino acid sequences, or of two nucleic acid sequences is defined as the percentage of amino acid residues or nucleotides in a candidate sequence that are identical with the amino acid residues or nucleotides in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

A variant may be created by introducing substitutions, additions, or deletions into a polynucleotide encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides comprising amino acid sequences sufficiently identical to or derived from the amino acid sequence of a native polypeptide which include fewer amino acids than the full length polypeptides. A portion of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally occurring allelic variant may contain conservative amino acid substitutions from the native polypeptide sequence or it may contain a substitution of an amino acid from a corresponding position in a polypeptide homolog, for example, a murine polypeptide.

A polypeptide variant may be identified by modifying a GAS marker polypeptide sequence and evaluating the antigenic properties of the modified polypeptide using for example, an immunohistochemical assay, an enzyme linked immunosorbant assay (ELISA), a radioimmunoassay (RIA) or a western blot assay.

A GAS marker polypeptide may comprise a biologically functional equivalent of at least about 5, 10, 15, 20, 25, 50, 100, 150 or 200 amino acids of a GAS marker polypeptide of Table 3, 4 or 5. A biologically equivalent polypeptide is a polypeptide that reacts substantially the same as a GAS marker polypeptide in an assay such as an immunohistochemcial assay, an ELISA, an RIA or western blot assay, i.e. it has 90-110% of the activity of the original polypeptide. In an aspect of a competition assay of the invention, the biologically equivalent polypeptide reduces binding of the polypeptide to a corresponding reactive antigen or antibody by about 80%, 85% 90%, 95%, 99% or 100%.

A GAS marker polypeptide includes truncated amino acid sequences preferably comprising or consisting essentially of at least one epitope. The truncated sequences can be used as reagents in methods of the invention or as subunit antigens in compositions for antiserum production or vaccines. Truncated sequences can be produced by various known treatments of native polypeptides or by making synthetic or recombinant polypeptides comprising a GAS marker polypeptide sequence. Polypeptides comprising truncated sequences can be made up entirely of GAS marker polypeptide sequences (one or more epitopes, either contiguous or noncontiguous), or GAS marker polypeptide sequences and heterologous sequences in a chimeric or fusion protein. Examples of heterologous sequences include sequences that provide for secretion from a recombinant host, enhance immunological reactivity of the GAS marker polypeptide epitopes or facilitate the coupling of the polypeptide to an immunoassay support or vaccine carrier. [See for example, U.S. Pat. Nos. 4,772,840 and 4,629,783 and EPO Publication Nos. 116201 and 259149.] The size of truncated GAS marker polypeptides can vary, but preferably the minimum size is a sequence sufficient to provide a GAS marker polypeptide epitope and the maximum size is not substantially greater than that required to provide the desired epitope. Generally, the truncated amino acid sequence ranges from about 5 to about 100 amino acids in length. In aspects of the invention, the sequence will be a maximum of about 50 amino acids in length, preferably a maximum of about 30 amino acids. It preferred aspects of the invention, sequences of at least about 10, 12, or 15 amino acids up to a maximum of about 20 to 25 amino acids are selected.

In aspects of the invention, in particular methods involving detecting antibodies specific for GAS marker polypeptides, the GAS marker polypeptides can comprise or consist essentially of one or more epitopes (i.e., antigenic determinants of the polypeptides). Epitopes include without limitation linear epitopes, sequential epitopes or conformational epitopes. An epitope could comprise amino acids in a spatial conformation which is unique to the epitope. Generally an epitope consists of at least 5 amino acids, and more usually consists of at least 8-10 amino acids. Epitopes within a GAS marker polypeptide can be identified by methods known in the art such as immunoassays. [See for example, the methods described in U.S. Pat. No. 4,554,101 and Jameson & Wolf, CABIOS 4:181-186 (1988).] By way of example, a GAS marker polypeptide can be isolated and screened, and a series of short peptides and overlapping peptides, which together span an entire polypeptide sequence, can be prepared by proteolytic cleavage. By starting with various polypeptide fragments, each fragment can be tested for the presence of epitopes recognized in an ELISA. For example, in an ELISA assay a GAS marker polypeptide, such as a 100-mer polypeptide fragment, can be attached to a solid support or carrier. Labeled antibodies are added to the solid support and allowed to bind to the unlabeled antigen fragments, under conditions where non-specific absorption is blocked, and any unbound antibody and other polypeptides are washed away. Antibody binding is detected by, for example, a reaction that converts a colorless substrate into a colored reaction product. Progressively smaller and overlapping fragments can then be tested to map an epitope of interest. A computer analysis of a GAS marker polypeptide sequence can also be carried out to identify potential epitopes and the oligopeptides can be prepared comprising the identified regions for screening.

A “chimeric protein” or “fusion protein” comprises all or part (preferably biologically active) of a GAS marker polypeptide operably linked to a heterologous polypeptide (i.e., a polypeptide other than a GAS marker polypeptide). Within the fusion protein, the term “operably linked” is intended to indicate that a GAS marker polypeptide and the heterologous polypeptide are fused in-frame to each other. The heterologous polypeptide can be fused to the N-terminus or C-terminus of a GAS marker polypeptide. A useful fusion protein is a GST fusion protein in which a GAS marker polypeptide is fused to the C-terminus of GST sequences. Chimeric and fusion proteins can be produced by standard recombinant DNA techniques.

A GAS marker polypeptide may be prepared by recombinant or synthetic methods, or isolated from a variety of sources, or by any combination of these and similar techniques. In aspects of the invention, a GAS marker polypeptide including truncations or fragments thereof can be produced recombinantly. A polynucleotide encoding a GAS marker polypeptide can be introduced into a recombinant expression vector, which can be expressed in a suitable expression host cell system using techniques well known in the art. A variety of bacterial, yeast, plant, mammalian, and insect expression systems are available in the art and any such expression system can be used. Optionally, a polynucleotide encoding a polypeptide can be translated in a cell-free translation system. A polypeptide can be chemically synthesized using standard techniques and equipment for preparing synthetic peptides. By way of example, the polypeptides/peptides may be prepared using a 9600 Millegen/Biosearch synthesizer or a 40 well multiple peptide synthesizer (MPS 396, Advanced Chem Tech, Louisville, Ky.), and purified by reverse HPLC and characterized by electrospray ionization spectrometry. A GAS marker polypeptide can also be obtained from GAS cells.

A GAS marker polynucleotide includes polynucleotides that encode a GAS marker polypeptide listed in Tables 3, 4 and 5 or a polynucleotide listed in Tables 3, 4 and 5. The polynucleotide markers include complementary nucleic acid sequences, and nucleic acids that are substantially identical to these sequences (e.g. having at least about 45%, 50%, 55%, 60%, 65%, 70%, 75%, 80%, 85%, 90%, 95%, 97%, 98%, or 99% sequence identity). Polynucleotide markers also include sequences that differ from a native sequence due to degeneracy in the genetic code. Polynucleotide markers also include nucleic acids that hybridize under stringent conditions, preferably high stringency conditions to a GAS polynucleotide marker. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

Polynucleotide markers also include truncated nucleic acids or nucleic acid fragments and variant forms of the nucleic acids that arise by alternative splicing of an mRNA corresponding to a DNA. A truncated polynucleotide marker or fragment can comprise about 5, 10, 15, 20, 25, 30, 35, 40, 50, 75, 100, 150 or 200 nucleotides. In aspects of the invention, the GAS marker polynucleotides are cloned fragments of GAS genes identified in Table 3, 4 or 5.

Polynucleotide markers are intended to include DNA and RNA (e.g. mRNA) and can be either double stranded or single stranded. A polynucleotide may, but need not, include additional coding or non-coding sequences, or it may, but need not, be linked to other molecules and/or carrier or support materials. The polynucleotide markers for use in the methods of the invention may be of any length suitable for a particular method. In certain applications the term refers to antisense polynucleotides (e.g. mRNA or DNA strand in the reverse orientation to sense polynucleotide markers). GAS marker polynucleotides include unmodified forms of the polynculeotides as well as known modifications, including without limitation, labels which are known in the art, methylation, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example proteins (including for e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc).

GAS marker polynucleotides can be cloned into an expression vector comprising regulatory elements (e.g. origins of replication, promoters, enhancers) that control expression of the polynucleotides in host cells. Examples of expression vectors include without limitation a plasmid, such as pBR322, pUC, or ColE1, an adenovirus vector, such as an adenovirus Type 2 vector or Type 5 vector, Sindbis virus, simian virus 40, alphavirus vectors, poxvirus vectors, cytomegalovirus, retroviral vectors, such as murine sarcoma virus, mouse mammary tumor virus, Moloney murine leukemia virus and Rous sarcoma virus. In addition, minichromosomes (e.g., MC and MC1), bacteriophages, phagemids, yeast artificial chromosomes, bacterial artificial chromosomes, virus particles, virus-like particles, cosmids and replicons can also be used.

GAS marker polynucleotides can be isolated from nucleic acid sequences present in samples, such as blood, serum, plasma, urine, feces, cerebrospinal fluid, amniotic fluid, wound exudate, or tissue from an infected subject. GAS marker polynucleotides can also be synthesized in the laboratory using automatic synthesizers or the polynucleotides can be amplified from either genomic DNA or cDNA encoding the polypeptides.

Statistically different levels”, “significantly altered levels”, or “significant difference” in levels of markers in a patient sample compared to a control or standard (e.g. normal levels or levels in other samples from a patient) may represent levels that are higher or lower than the standard error of the detection assay. In particular embodiments, the levels may be 1.5, 2, 3, 4, 5, or 6 times higher or lower than the control or standard.

“Microarray” and “array,” refer to nucleic acid or nucleotide arrays or protein or peptide arrays that can be used to detect biomolecules associated with a GAS disease, for instance to measure gene expression. A variety of arrays are made in research and manufacturing facilities worldwide, some of which are available commercially. By way of example, spotted arrays and in situ synthesized arrays are two kinds of nucleic acid arrays that differ in the manner in which the nucleic acid materials are placed onto the array substrate. A widely used in situ synthesized oligonucleotide array is GeneChip™ made by Affymetrix, Inc. Oligonucleotide probes that are 20- or 25-bases long can be synthesized in silico on the array substrate. These arrays can achieve high densities (e.g., more than 40,000 genes per cm²). Generally spotted arrays have lower densities, but the probes, typically partial cDNA molecules, are much longer than 20- or 25-mers. Examples of spotted cDNA arrays include LifeArray made by Incyte Genomics and DermArray made by IntegriDerm (or Invitrogen). Pre-synthesized and amplified cDNA sequences are attached to the substrate of spotted arrays. Protein and peptide arrays also are known (see for example, Zhu et al., Science 293:2101 (2001).

“Binding agent” refers to a substance such as a polypeptide or antibody that specifically binds to one or more GAS markers. A substance “specifically binds” to one or more GAS markers if it reacts at a detectable level with one or more GAS markers, and does not react detectably with peptides containing an unrelated or different sequence. Binding properties may be assessed using an ELISA, which may be readily performed by those skilled in the art (see for example, Newton et al, Develop. Dynamics 197: 1-13, 1993).

A binding agent may be a ribosome, with or without a peptide component, an aptamer, an RNA molecule, or a polypeptide. A binding agent may be a polypeptide that comprises one or more GAS marker sequence, a peptide variant thereof, or a non-peptide mimetic of such a sequence.

An aptamer includes a DNA or RNA molecule that binds to nucleic acids and proteins. An aptamer that binds to a protein (or binding domain) of a GAS marker can be produced using conventional techniques, without undue experimentation. (For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)).

Antibodies for use in the present invention include but are not limited to monoclonal antibodiesm, polyclonal antibodies, immunologically active fragments (e.g. Fab, (Fab)₂ Fab′, and Fav′-SH fragments), antibody heavy chains, humanized antibodies, antibody light chains, genetically engineered single chain F, molecules (Ladner et al, U.S. Pat. No. 4,946,778), chimeric antibodies, for example, antibodies which contain the binding specificity of murine antibodies, but in which the remaining portions are of human origin, or derivatives, such as enzyme conjugates or labelled derivatives. An antibody can be any antibody class, including IgG, IgM, IgA, IgD and IgE. In an embodiment of the invention, antibodies are reactive against a GAS marker if they bind with a Kₐ of greater than or equal to 10⁻⁷ M.

Antibodies including monoclonal and polyclonal antibodies, fragments and chimeras, may be prepared using methods known to those skilled in the art. Isolated native or recombinant GAS markers may be utilized to prepare antibodies. An antibody can be made in vivo in suitable laboratory animals or in vitro using recombinant procedures or chemical techniques. (See, for example, Kohler et al. (1975) Nature 256:495-497; Kozbor et al. (1985) J. Immunol. Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; Pound (1998) Immunochemical Protocols, Humana Press, Totowa, N.J. for the preparation of phagemid or B-lymphocyte immunoglobulin libraries to identify antibodies; U.S. Pat. No. 4,676,980 for methods for chemically constructing antibodies; U.S. Pat. No. 5,482,856, Jones et al, Nature 321:522, 1986; Reichmann et al., Nature 332:323, 1988, and Presta, Curr. Op Struct. Biol. 2:593 1992, for methods for producing chimeric antibodies). Antibodies specific for a GAS marker may also be obtained from scientific or commercial sources.

Antibodies against GAS marker polypeptides comprising epitopes can also be readily produced. By way of example, hybridomas producing antibodies specific for GAS marker polypeptides derived from normal B cells obtained from a mammal immunized with GAS marker polypeptides can be identified using RIA or ELISA and isolated by cloning or limited dilution. The clones can be further screened to identify clones producing antibodies specific for GAS marker polypeptides. Monoclonal antibodies can be screened for specificity using procedures known in the art such as an ELISA. Isotopes of monoclonal antibodies can be selected from an initial fusion or prepared from a parental hybridoma secreting a different isotype using a sib selection technique to isolate class-switch variants. [See, for example, Steplewski et al, PNAS USA 82:8653, 1985 and Spria et al, J. Immunolog. Meth. 74:307, 1984.]

### Markers

The invention provides a set of markers correlated with GAS disease. A set of these markers identified as useful for detection, diagnosis, prevention and therapy of GAS disease comprises 1, 2, 3, 4, 5, 6, 7, 8, 9, 10, 15, 20 or more of the polynucleotides and polypeptides listed in Tables 3, 4 and 5. The invention provides a marker set that distinguish GAS disease and uses therefore comprising or consisting of one or more polypeptides or polynucleotides listed in Tables 3, 4 and 5.

In an aspect, the invention provides a method for classifying a GAS disease comprising detecting a difference in the expression of a first plurality of GAS markers relative to a control, the first plurality of GAS markers consisting of one or more polypeptides or polynucleotides listed in Tables 3, 4 and 5. In an aspect, the control comprises markers derived from a pool of samples from individual patients with no GAS disease.

Any of the markers provided herein may be used alone or with other markers of GAS disease, or with markers for other phenotypes or conditions.

### Nucleic Acid Methods/Assays

As noted herein a GAS disease may be detected based on the amount/level of GAS marker polynucleotides in a sample. Techniques for detecting polynucleotides such as polymerase chain reaction (PCR) and hybridization assays are well known in the art.

Probes may be used in hybridization techniques to detect GAS marker polynucleotides. The technique generally involves contacting and incubating nucleic acids (e.g. recombinant DNA molecules, cloned genes) obtained from a sample from a patient or other cellular source with a probe under conditions favorable for the specific annealing of the probes to complementary sequences in the nucleic acids. After incubation, the non-annealed nucleic acids are removed, and the presence of nucleic acids that have hybridized to the probe if any are detected.

Nucleotide probes for use in the detection of nucleic acid sequences in samples may be constructed using conventional methods known in the art. Suitable probes may be based on nucleic acid sequences encoding at least 5 sequential amino acids from regions of a GAS marker, preferably they comprise 10-200, more particularly 10-30, 10-40, 20-50, 40-80, 50-150, 80-120 nucleotides in length.

The probes may comprise DNA or DNA mimics (e.g., derivatives and analogues) corresponding to a portion of an organism's genome, or complementary RNA or RNA mimics. Mimics are polymers comprising subunits capable of specific, Watson-Crick-like hybridization with DNA, or of specific hybridization with RNA. The nucleic acids can be modified at the base moiety, at the sugar moiety, or at the phosphate backbone.

DNA can be obtained using standard methods such as polymerase chain reaction (PCR) amplification of genomic DNA or cloned sequences. (See, for example, in Innis et al., eds., 1990, PCR Protocols: A Guide to Methods and Applications, Academic Press Inc., San Diego, Calif.). Computer programs known in the art can be used to design primers with the required specificity and optimal amplification properties, such as Oligo version 5.0 (National Biosciences). Controlled robotic systems may be useful for isolating and amplifying nucleic acids.

A nucleotide probe may be labeled with a detectable substance such as a radioactive label that provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect GAS marker polynucleotides. The nucleotide probes may also be useful in the diagnosis of a GAS disease involving one or more GAS markers, in monitoring the progression of such disorder, or monitoring a therapeutic treatment.

The detection of GAS marker polynucleotides may involve the amplification of specific gene sequences using an amplification method such as polymerase chain reaction (PCR), followed by the analysis of the amplified molecules using techniques known to those skilled in the art. Suitable primers can be routinely designed by one of skill in the art.

By way of example, at least two oligonucleotide primers may be employed in a PCR based assay to amplify a portion of a polynucleotide encoding one or more GAS marker derived from a sample, wherein at least one of the oligonucleotide primers is specific for (i.e. hybridizes to) a polynucleotide encoding the GAS marker. The amplified cDNA is then separated and detected using techniques well known in the art, such as gel electrophoresis.

In order to maximize hybridization under assay conditions, primers and probes employed in the methods of the invention generally have at least about 60%, preferably at least about 75%, and more preferably at least about 90% identity to a portion of a polynucleotide encoding a GAS marker; that is, they are at least 10 nucleotides, and preferably at least 20 nucleotides in length. In an embodiment the primers and probes are at least about 10-40 nucleotides in length.

Hybridization and amplification techniques described herein may be used to assay qualitative and quantitative aspects of GAS marker polynucleotide expression. For example, RNA may be isolated from a cell type or tissue known to express a GAS marker polynucleotide and tested utilizing the hybridization (e.g. standard Northern analyses) or PCR techniques referred to herein.

The primers and probes may be used in the above-described methods in situ i.e. directly on tissue sections (fixed and/or frozen) of patient tissue obtained from biopsies or resections.

In an aspect of the invention, a method is provided employing reverse transcriptase-polymerase chain reaction (RT-PCR), in which PCR is applied in combination with reverse transcription. Generally, RNA is extracted from a sample tissue using standard techniques (for example, guanidine isothiocyanate extraction as described by Chomcynski and Sacchi, Anal. Biochem. 162:156-159, 1987) and is reverse transcribed to produce cDNA. The cDNA is used as a template for a polymerase chain reaction. The cDNA is hybridized to a set of primers, at least one of which is specifically designed against a GAS marker sequence. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with a suspected GAS disease and an individual who is not afflicted with a GAS disease. The reaction may be performed on several dilutions of cDNA spanning at least two orders of magnitude. A statistically significant difference in expression in several dilutions of the subject sample as compared to the same dilutions of the non-disease sample may be considered positive for the presence of a GAS disease.

In an embodiment, the invention provides methods for determining the presence or absence of a GAS disease in a subject comprising (a) contacting a sample obtained from the subject with oligonucleotides that hybridize to GAS marker polynucleotides; and (b) detecting in the sample a level of nucleic acids that hybridize to the polynucleotides relative to a predetermined cut-off value, and therefrom determining the presence or absence of a GAS disease in the subject. In an aspect, the GAS marker polynucleotides are one or more of the polynucleotides listed in Tables 3, 4 and 5.

The invention provides a method wherein an GAS marker mRNA is detected by (a) isolating mRNA from a sample and combining the mRNA with reagents to convert it to cDNA; (b) treating the converted cDNA with amplification reaction reagents and nucleic acid primers that hybridize to one or more GAS marker polynucleotides, to produce amplification products; (d) analyzing the amplification products to detect amounts of mRNA encoding GAS markers; and (e) comparing the amount of mRNA to an amount detected against a panel of expected values for normal subjects derived using similar nucleic acid primers.

GAS marker-positive samples or alternatively higher levels in patients compared to a control (e.g. non-infected individual) may be indicative of disease, late stage disease, and/or that the patient is not responsive to therapy.

In another embodiment, the invention provides methods for diagnosing or determining the presence or absence of a GAS disease in a subject comprising (a) contacting a sample obtained from the subject with oligonucleotides that hybridize to one or more GAS marker polynucleotides; and (b) detecting in the sample levels of nucleic acids that hybridize to the oligonucleotides relative to a predetermined cut-off value, and therefrom determining the presence or absence of a GAS disease in the subject.

In particular, the invention provides a method wherein a GAS marker mRNA is detected by (a) isolating mRNA from a sample and combining the mRNA with reagents to convert it to cDNA; (b) treating the converted cDNA with amplification reaction reagents and nucleic acid primers that hybridize to the GAS marker to produce amplification products; (d) analyzing the amplification products to detect an amount of the GAS marker mRNA; and (e) comparing the amount of mRNA to an amount detected against a panel of expected values for healthy individuals derived using similar nucleic acid primers.

Marker-positive samples or alternatively higher levels, in particular significantly higher levels of a GAS marker in patients compared to a control (e.g. normal) are indicative of a GAS disease.

Oligonucleotides or longer fragments derived from GAS marker polynucleotides may be used as targets in a microarray. The microarray can be used to simultaneously monitor the expression levels of large numbers of genes and to identify genetic variants and mutations. The information from the microarray may be used to determine gene function, to diagnose a disorder, and to develop and monitor the activities of therapeutic agents.

The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505; Heller, R. A. et al. (1997) Proc. Natl. Acad. Sci. 94:2150-2155; and Heller, M. J. et al. (1997) U.S. Pat. No. 5,605,662.)

Thus, the invention also includes an array comprising one or more GAS marker polynucleotides and optionally other markers. The array can be used to assay expression of GAS marker polynucleotides in the array. The invention allows the quantitation of expression of one or more GAS marker polynucleotides.

Microarrays typically contain at separate sites nanomolar quantities of individual genes, cDNAs, or ESTs on a substrate (e.g., nitrocellulose or silicon plate), or photolithographically prepared glass substrate. The arrays are hybridized to cDNA probes using conventional techniques with gene-specific primer mixes. The target polynucleotides to be analyzed are isolated, amplified and labeled, typically with fluorescent labels, radiolabels or phosphorous label probes. After hybridization is completed, the array is inserted into the scanner, where patterns of hybridization are detected. Data are collected as light emitted from the labels incorporated into the target, which becomes bound to the probe array. Probes that completely match the target generally produce stronger signals than those that have mismatches. The sequence and position of each probe on the array are known, and thus by complementarity, the identity of the target nucleic acid applied to the probe array can be determined.

Microarrays are prepared by selecting polynucleotide probes and immobilizing them to a solid support or surface. The probes may comprise DNA sequences, RNA sequences, copolymer sequences of DNA and RNA, DNA and/or RNA analogues, or combinations thereof. The probe sequences may be full or partial fragments of genomic DNA, or they may be synthetic oligonucleotide sequences synthesized either enzymatically in vivo, enzymatically in vitro (e.g., by PCR), or non-enzymatically in vitro.

The probe or probes used in the methods of the invention can be immobilized to a solid support or surface which may be either porous or non-porous. For example, the probes can be attached to a nitrocellulose or nylon membrane or filter covalently at either the 3′ or the 5′ end of the polynucleotide probe. The solid support may be a glass or plastic surface. In an aspect of the invention, hybridization levels are measured to microarrays of probes consisting of a solid support on the surface of which are immobilized a population of polynucleotides, such as a population of DNA or DNA mimics, or, alternatively, a population of RNA or RNA mimics. A solid support may be a nonporous or, optionally, a porous material such as a gel.

In accordance with embodiments of the invention, a microarray is provided comprising a support or surface with an ordered array of hybridization sites or “probes” each representing one of the markers described herein. The microarrays can be addressable arrays, and in particular positionally addressable arrays. Each probe of the array is typically located at a known, predetermined position on the solid support such that the identity of each probe can be determined from its position in the array. In preferred embodiments, each probe is covalently attached to the solid support at a single site.

Microarrays used in the present invention are preferably (a) reproducible, allowing multiple copies of a given array to be produced and easily compared with each other; (b) made from materials that are stable under hybridization conditions; (c) small, (e.g., between 1 cm² and 25 cm², between 12 cm² and 13 cm², or 3 cm²; and (d) comprise a unique set of binding sites that will specifically hybridize to the product of a single gene in a cell (e.g., to a specific mRNA, or to a specific cDNA derived therefrom). However, it will be appreciated that larger arrays may be used particularly in screening arrays, and other related or similar sequences will cross hybridize to a given binding site.

In accordance with an aspect of the invention, the microarray is an array in which each position represents one of the GAS marker polynucleotides described herein. Each position of the array can comprise a DNA or DNA analogue based on genomic DNA to which a particular RNA or cDNA transcribed from a genetic marker can specifically hybridize. A DNA or DNA analogue can be a synthetic oligomer or a gene fragment. In an embodiment, probes representing each of the GAS markers is present on the array.

Probes for the microarray can be synthesized using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 10 and about 500 bases, 20-100 bases, or 40-70 bases in length. Synthetic nucleic acid probes can include non-natural bases, such as, without limitation, inosine. Nucleic acid analogues such as peptide nucleic acid may be used as binding sites for hybridization. (see, e.g., Egholm et al., 1993, Nature 363:566-568; U.S. Pat. No. 5,539,083).

Probes can be selected using an algorithm that takes into account binding energies, base composition, sequence complexity, cross-hybridization binding energies, and secondary structure (see Friend et al., International Patent Publication WO 01/05935, published Jan. 25, 2001).

Positive control probes, (e.g., probes known to be complementary and hybridize to sequences in the target polynucleotides), and negative control probes, (e.g., probes known to not be complementary and hybridize to sequences in the target polynucleotides) are typically included on the array. Positive controls can be synthesized along the perimeter of the array or synthesized in diagonal stripes across the array. A reverse complement for each probe can be next to the position of the probe to serve as a negative control.

The probes can be attached to a solid support or surface, which may be made from glass, plastic (e.g., polypropylene, nylon), polyacrylamide, nitrocellulose, gel, or other porous or nonporous material. The probes can be printed on surfaces such as glass plates (see Schena et al., 1995, Science 270:467-470). This method may be particularly useful for preparing microarrays of cDNA (See also, DeRisi et al., 1996, Nature Genetics 14:457-460; Shalon et al., 1996, Genome Res. 6:639-645; and Schena et al., 1995, Proc. Natl. Acad. Sci. U.S.A. 93:10539-11286).

High-density oligonucleotide arrays containing thousands of oligonucleotides complementary to defined sequences, at defined locations on a surface can be produced using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). Using these methods oligonucleotides (e.g., 60-mers) of known sequence are synthesized directly on a surface such as a derivatized glass slide. The array produced may be redundant, with several oligonucleotide molecules per RNA.

Microarrays can be made by other methods including masking (Maskos and Southern, 1992, Nuc. Acids. Res. 20:1679-1684). In an embodiment, microarrays of the present invention are produced by synthesizing polynucleotide probes on a support wherein the nucleotide probes are attached to the support covalently at either the 3′ or the 5′ end of the polynucleotide.

The invention provides microarrays comprising a disclosed marker set. In one embodiment, the invention provides a microarray for distinguishing GAS disease samples comprising a positionally-addressable array of polynucleotide probes bound to a support, the polynucleotide probes comprising a plurality of polynucleotide probes of different nucleotide sequences, each of the different nucleotide sequences comprising a sequence complementary and hybridizable to a plurality of genes, the different nucleotide sequences selected from the group consisting of the polynucleotides listed in Tables 3, 4 and 5.

The invention provides gene marker sets that distinguish GAS disease and uses thereof. In an aspect, the invention provides a method for classifying a GAS disease comprising detecting a difference in the expression of a first plurality of genes relative to a control, the first plurality of genes consisting of 2, 3, 4, 5, 6, 7, 8, 9, 10, 15, 20 or 25 or more of the genes listed in Tables 3, 4 and 5. In another specific aspect, the control comprises nucleic acids derived from a pool of samples from individual control patients.

### Protein Methods

Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of a GAS disease in a subject may be determined by (a) contacting a sample from the subject with a binding agent; (b) detecting in the sample a level of a GAS marker polypeptide that binds to the binding agent; and (c) comparing the level of protein with a predetermined standard or cut-off value.

In particular embodiments of the invention, the binding agent is an antibody. Antibodies specifically reactive with one or more GAS marker polypeptide, or derivatives, such as enzyme conjugates or labeled derivatives, may be used to detect one or more GAS marker polypeptide in various samples (e.g. biological materials). They may be used as diagnostic or prognostic reagents and they may be used to detect abnormalities in the levels of one or more GAS marker polypeptide, and/or temporal, tissue, cellular, or subcellular location of one or more GAS marker polypeptide. Antibodies may also be used to screen potentially therapeutic compounds in vitro to determine their effects on GAS diseases involving one or more GAS marker proteins and other conditions. In vitro immunoassays may also be used to assess or monitor the efficacy of particular therapies.

In an aspect, the invention provides a method for monitoring or diagnosing a GAS disease in a subject by quantitating one or more GAS marker polypeptides in a biological sample from the subject comprising reacting the sample with antibodies specific for one or more GAS marker polypeptides, which are directly or indirectly labeled with detectable substances and detecting the detectable substances. In a particular embodiment of the invention, GAS marker polypeptides are quantitated or measured.

In an aspect of the invention, a method for diagnosing or detecting a GAS disease is provided comprising:
(a) obtaining a sample suspected of containing one or more GAS marker polypeptides associated with a GAS disease;
(b) contacting said sample with antibodies that specifically bind to the GAS marker polypeptides under conditions effective to bind the antibodies and form complexes;
(c) measuring the amount of GAS marker polypeptides present in the sample by quantitating the amount of the complexes; and
(d) comparing the amount of GAS marker polypeptides present in the samples with the amount of GAS marker polypeptides in a control, wherein a change or significant difference in the amount of GAS marker polypeptides in the sample compared with the amount in the control is indicative of a GAS disease.

In an embodiment, the invention contemplates a method for monitoring the progression of a GAS disease in an individual, comprising:
(a) contacting antibodies which bind to one or more GAS marker polypeptides with a sample from the individual so as to form complexes comprising the antibodies and one or more GAS marker polypeptides in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of disease, disease stage, and/or progression of the disease in said individual.

The amount of complexes may also be compared to a value representative of the amount of the complexes from an individual not afflicted with a GAS disease at different stages. A significant difference in complex formation may be indicative of advanced disease or an unfavourable prognosis.

In embodiments of the methods of the invention, selected GAS markers detected in samples and higher levels, in particular significantly higher levels, compared to a control (e.g. normal) is indicative of a GAS disease.

Antibodies may be used to detect and quantify one or more GAS marker polypeptides in a sample in order to diagnose and treat a GAS disease. Immunohistochemical methods for the detection of antigens in tissue samples are well known in the art. For example, immunohistochemical methods are described in Taylor, Arch. Pathol. Lab. Med. 102:112 (1978). Briefly, in the context of the present invention, a tissue sample obtained from a subject suspected of having a GAS disease is contacted with antibodies, preferably monoclonal antibodies recognizing one or more GAS marker polypeptides. The site at which the antibodies are bound is determined by selective staining of the sample by standard immunohistochemical procedures. The same procedure may be repeated on the same sample using other antibodies that recognize one or more GAS marker polypeptides. Alternatively, a sample may be contacted with antibodies against one or more GAS marker polypeptides simultaneously, provided that the antibodies are labeled differently or are able to bind to a different label.

Antibodies may be used in any known immunoassays that rely on the binding interaction between antigenic determinants of one or more GAS marker polypeptide and the antibodies. Immunoassay procedures for in vitro detection of antigens in fluid samples are also well known in the art. [See for example, Paterson et al., Int. J. Can. 37:659 (1986) and Burchell et al., Int. J. Can. 34:763 (1984) for a general description of immunoassay procedures]. Qualitative and/or quantitative determinations of one or more GAS marker in a sample may be accomplished by competitive or non-competitive immunoassay procedures in either a direct or indirect format. Detection of one or more GAS marker polypeptide using antibodies can be done utilizing immunoassays which are run in either the forward, reverse or simultaneous modes. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. These terms are well understood by those skilled in the art. A person skilled in the art will know, or can readily discern, other immunoassay formats without undue experimentation.

According to an embodiment of the invention, an immunoassay for detecting one or more GAS marker polypeptides in a biological sample comprises contacting binding agents that specifically bind to GAS marker polypeptides in the sample under conditions that allow the formation of first complexes comprising a binding agent and GAS marker polypeptides and determining the presence or amount of the complexes as a measure of the amount of GAS marker polypeptides contained in the sample. In a particular embodiment, the binding agents are labeled differently or are capable of binding to different labels.

An antibody microarray in which binding sites comprise immobilized, preferably monoclonal, antibodies specific to a substantial fraction of GAS marker polypeptides of interest can be utilized in the present invention. Antibody arrays can be prepared using methods known in the art [(see for example, Zhu et al., Science 293:2101 (2001) and reference 20].

Antibodies specific for one or more GAS marker polypeptides may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, and acetylcholinesterase), biotinyl groups (which can be detected by marked avidin e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

One of the ways an antibody can be detectably labeled is to link it directly to an enzyme. The enzyme when later exposed to its substrate will produce a product that can be detected. Examples of detectable substances that are enzymes are horseradish peroxidase, beta-galactosidase, luciferase, alkaline phosphatase, acetylcholinesterase, malate dehydrogenase, ribonuclease, urease, catalase, glucose-6-phosphate, staphylococcal nuclease, delta-5-steriod isomerase, yeast alcohol dehydrogenase, alpha-glycerophosphate, triose phosphate isomerase, asparaginase, glucose oxidase, and acetylcholine esterase.

For increased sensitivity in an immunoassay system a fluorescence-emitting metal atom such as Eu (europium) and other lanthanides can be used. These can be attached to the desired molecule by means of metal-chelating groups such as DTPA or EDTA.

A bioluminescent compound may also be used as a detectable substance. Bioluminescence is a type of chemiluminescence found in biological systems where a catalytic protein increases the efficiency of the chemiluminescent reaction. The presence of a bioluminescent molecule is determined by detecting the presence of luminescence. Examples of bioluminescent detectable substances are luciferin, luciferase and aequorin.

Indirect methods may also be employed in which the primary antigen-antibody reaction is amplified by the introduction of a second antibody, having specificity for the antibody reactive against one or more GAS marker polypeptides. By way of example, if the antibody having specificity against one or more GAS marker polypeptides is a rabbit IgG antibody, the second antibody may be goat anti-rabbit gamma-globulin labelled with a detectable substance as described herein.

Methods for conjugating or labelling the antibodies discussed above may be readily accomplished by one of ordinary skill in the art. (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988 re methods for conjugating or labelling the antibodies with enzyme or ligand binding partner).

Cytochemical techniques known in the art for localizing antigens using light and electron microscopy may be used to detect one or more GAS marker polypeptides. Generally, antibodies may be labeled with detectable substances and one or more GAS marker polypeptides may be localised in tissues and cells based upon the presence of the detectable substances.

In the context of the methods of the invention, the sample, binding agents (e.g. antibodies specific for one or more GAS marker polypeptides), or one or more GAS marker polypeptides may be immobilized on a carrier, substrate or support. Examples of suitable carriers, substrates or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. A carrier, support or substrate can comprise microtiter wells, magnetic beads, non-magnetic beads, columns, matrices, membranes, fibrous mats composed of synthetic or natural fibers (e.g., glass or cellulose-based materials or thermoplastic polymers, such as, polyethylene, polypropylene, or polyester), sintered structures composed of particulate materials (e.g., glass or various thermoplastic polymers), or cast membrane films composed of nitrocellulose, nylon, polysulfone or the like (generally synthetic in nature. The support material may have any possible configuration including spherical (e.g. bead), cylindrical (e.g. inside surface of a test tube or well, or the external surface of a rod), or flat (e.g. sheet, test strip). Thus, the carrier, substrate or support may be in the shape of, for example, a tube, test plate, well, beads, disc, sphere, film, sheet, etc. A support or substrate material may be coated onto or bonded or laminated to appropriate inert carriers, such as paper, glass, plastic films, or fabrics. The immobilized sample, binding agents (e.g. antibodies specific for one or more GAS marker polypeptides), or one or more GAS marker polypeptides may be prepared by reacting the material with a suitable insoluble carrier using known chemical or physical methods, for example, cyanogen bromide coupling. An antibody may be indirectly immobilized using a second antibody specific for the antibody. For example, mouse antibody specific for a GAS marker may be immobilized using sheep anti-mouse IgG Fc fragment specific antibody coated on the carrier or support.

Where a radioactive label is used as a detectable substance, one or more GAS marker polypeptides may be localized by radioautography. The results of radioautography may be quantitated by determining the density of particles in the radioautographs by various optical methods, or by counting the grains.

Time-resolved fluorometry may be used to detect a signal. For example, the method described in Christopoulos TK and Diamandis EP Anal Chem 1992:64:342-346 may be used with a conventional time-resolved fluorometer.

In accordance with an embodiment of the invention, a method is provided wherein one or more GAS marker polypeptides antibodies are directly or indirectly labelled with enzymes, substrates for the enzymes are added wherein the substrates are selected so that the substrates, or a reaction product of an enzyme and substrate, form fluorescent complexes with a lanthanide metal (e.g. europium, terbium, samarium, and dysprosium, preferably europium and terbium). A lanthanide metal is added and one or more GAS markers are quantitated in the sample by measuring fluorescence of the fluorescent complexes. Enzymes are selected based on the ability of a substrate of the enzyme, or a reaction product of the enzyme and substrate, to complex with lanthanide metals such as europium and terbium. Suitable enzymes and substrates that provide fluorescent complexes are described in U.S. Pat. No. 5,312,922 to Diamandis. Examples of suitable enzymes include alkaline phosphatase and β-galactosidase. Preferably, the enzyme is alkaline phosphatase.

Examples of enzymes and substrates for enzymes that provide such fluorescent complexes are described in U.S. Pat. No. 5,312,922 to Diamandis. By way of example, when the antibody is directly or indirectly labelled with alkaline phosphatase the substrate employed in the method may be 4-methylumbelliferyl phosphate, 5-fluorosalicyl phosphate, or diflunisal phosphate. The fluorescence intensity of the complexes is typically measured using a time-resolved fluorometer e.g. a CyberFluor 615 Immunoanalyzer (Nordion International; Kanata, Ontario).

One or more GAS marker polypeptides antibodies may also be indirectly labelled with an enzyme. For example, the antibodies may be conjugated to one partner of a ligand binding pair, and the enzyme may be coupled to the other partner of the ligand binding pair. Representative examples include avidin-biotin, and riboflavin-riboflavin binding protein. In an embodiment, the antibodies are biotinylated, and the enzyme is coupled to streptavidin. In another embodiment, an antibody specific for a GAS marker polypeptide antibody is labeled with an enzyme.

In accordance with an embodiment, the present invention provides means for determining one or more GAS marker polypeptides in a sample by measuring one or more GAS marker polypeptides by immunoassay. It will be evident to a skilled artisan that a variety of immunoassay methods can be used to measure one or more GAS marker polypeptides. In general, an immunoassay method may be competitive or noncompetitive. Competitive methods typically employ an immobilized or immobilizable antibody to one or more GAS marker polypeptides and a labeled form of one or more GAS marker polypeptides. Sample GAS marker polypeptides and labeled GAS marker polypeptides compete for binding to antibodies to GAS marker polypeptides. After separation of the resulting labeled GAS marker polypeptides that have become bound to antibodies (bound fraction) from that which has remained unbound (unbound fraction), the amount of the label in either bound or unbound fraction is measured and may be correlated with the amount of GAS marker polypeptides in the test sample in any conventional manner, e.g., by comparison to a standard curve.

In an aspect, a non-competitive method is used for the determination of one or more GAS marker polypeptides, with the most common method being the “sandwich” method. In this assay, two antibodies to GAS marker polypeptides are employed. One of the antibodies to GAS marker polypeptides is directly or indirectly labeled (sometimes referred to as the “detection antibody”) and the other is immobilized or immobilizable (sometimes referred to as the “capture antibody”). The capture and detection antibodies can be contacted simultaneously or sequentially with the test sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally it is measured in the capture antibody phase since it comprises GAS markers bound by (“sandwiched” between) the capture and detection antibodies. In an embodiment, the label may be measured without separating the capture antibodies and liquid test mixture.

In a typical two-site immunometric assay for GAS marker polypeptides, one or both of the capture and detection antibodies are polyclonal antibodies or one or both of the capture and detection antibodies are monoclonal antibodies (i.e. polyclonal/polyclonal, monoclonal/monoclonal, or monoclonal/polyclonal). The label used in the detection antibody can be selected from any of those known conventionally in the art. The label may be an enzyme or a chemiluminescent moiety, but it can also be a radioactive isotope, a fluorophor, a detectable ligand (e.g., detectable by a secondary binding by a labeled binding partner for the ligand), and the like. In a particular aspect, the antibody is labelled with an enzyme which is detected by adding a substrate that is selected so that a reaction product of the enzyme and substrate forms fluorescent complexes. The capture antibody may be selected so that it provides a means for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody may comprise an antibody covalently or noncovalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, the capture antibody may be immobilized using a species specific antibody for the capture antibody that is bound to the solid phase.

Antibodies specific for a GAS marker polypeptide may be used to isolate GAS organisms or GAS marker antigens by immunoaffinity columns. The antibodies may be conjugated to a substrate, support or carrier for example by adsorption or covalent linkage with or without a spacer group. The immobilized antibodies can be used to bind GAS organisms or GAS marker antigens from a sample. The GAS organisms or antigens can be recovered from the substrate, support or carrier using methods known to a skilled artisan (e.g., change in pH).

GAS markers can be used to detect antibodies or antibody fragments specific for GAS markers or a GAS disease in a test sample. In aspects of the invention, the sample is a biological sample including, for example, sera, blood, cells, plasma, or tissue from a mammal such as a horse, cat, dog or human. A test sample can be untreated, precipitated, fractionated, separated, diluted, concentrated, or purified before combining with a GAS marker polypeptide. Generally, the methods comprise contacting a GAS marker polypeptide with a test sample under conditions that allow a polypeptide/antibody complex or an immunocomplex, to form. A GAS marker polypeptide specifically binds to an antibody specific for a GAS marker polypeptide located in the sample.

Methods (i.e., assays) and conditions that can be used to detect antibody/polypeptide complex binding or immunocomplexes are known to persons skilled in the art and are discussed herein. For example, a method of the invention for detecting antibodies can comprise an assay selected from the group consisting of a microtiter plate assay, a reversible flow chromatographic binding assay, a lateral flow immunoassay, an enzyme linked immunosorbent assay, a radioimmunoassay, a hemaglutination assay, a western blot assay, a fluorescence polarization immunoassay and an indirect immunofluorescence assay.

Assays can use supports, substrates or carriers or can be performed by immunoprecipitation or any other methods that do not utilize solid phases. Where a solid phase is used, a GAS marker polypeptide is directly or indirectly attached to a solid support, substrate or a carrier described herein, for example, a microtiter well, magnetic bead, non-magnetic bead, column, matrix, membrane, fibrous mat composed of synthetic or natural fibers, sintered structure composed of particulate materials, or cast membrane film composed of nitrocellulose, nylon, polysulfone or the like. In an aspect of the invention, one or more GAS marker polypeptides are coated on a solid phase or substrate. A test sample suspected of containing an anti-GAS marker polypeptide antibody or fragment thereof is incubated with an indicator reagent comprising a detectable substance or label conjugated to an antibody or antibody fragment specific for a GAS marker polypeptide for a time and under conditions sufficient to form antigen/antibody complexes of either antibodies of the test sample to the GAS marker polypeptides of the solid phase or the indicator reagent conjugated to an antibody specific for GAS marker polypeptides to the GAS marker polypeptides of the solid phase. The reduction in binding of the indicator reagent to the solid phase can be quantitatively measured. A measurable reduction in the signal compared to the signal generated from a confirmed negative test sample indicates the presence of antibody to the GAS marker polypeptides in the test sample. This type of assay can quantitate the amount of antibodies specific for GAS marker polypeptides in a test sample.

In another aspect, one or more GAS marker polypeptides are coated onto a support or substrate and a GAS marker polypeptide is conjugated to a detectable substance or label and added to a test sample. This mixture is applied to the support or substrate. If GAS marker polypeptide antibodies are present in the test sample they will bind the polypeptide conjugated to the detectable substance and to the GAS marker polypeptide immobilized on the support. The polypeptide/antibody/detectable complex can then be detected. This type of assay can quantitate the amount of antibodies specific for GAS marker polypeptides in a test sample.

In another aspect, one or more GAS marker polypeptides are coated onto a support or substrate and the test sample is applied to the support or substrate and incubated. The solid support is washed to remove unbound components from the sample. If antibodies specific for GAS marker polypeptides are present in the test sample, they will bind to the polypeptide coated on the solid phase. This polypeptide/antibody complex can be detected using a second species-specific antibody that is conjugated to a detectable substance. The polypeptide/antibody/anti-species antibody detectable substance complex can then be detected. This type of assay can quantitate the amount of antibodies specific for GAS marker polypeptides in a test sample.

The formation of a polypeptide/antibody complex or a polypeptide/antibody/detectable substance complex can be detected by radiometric, colormetric, fluorometric, size-separation, or precipitation methods. A polypeptide/antibody complex can also be detected by the addition of a secondary antibody that is coupled to a detectable substance. Indicator reagents comprising detectable substances (labels) associated with an immunocomplex can be detected using the methods described above and include chromogenic agents, catalysts such as enzyme conjugates fluorescent compounds such as fluorescein and rhodamine, chemiluminescent compounds such as dioxetanes, acridiniums, phenanthridiniums, ruthenium, and luminol, radioactive elements, direct visual labels, as well as cofactors, inhibitors, magnetic particles, and the like.

The formation of a polypeptide/antibody complex is indicative of the presence of antibodies specific for GAS marker polypeptides in a test sample and therefore the methods of the invention can be used to diagnose GAS diseases or infections in a subject. The methods of the invention can also indicate the amount or quantity of antibodies specific for GAS marker polypeptides in a test sample. The amount of antibody present can be proportional to the signal generated, in particular the signal generated with indicator reagents where the detectable substance is an enzyme.

The above-described immunoassay methods and formats are intended to be exemplary and are not limiting.

### Computer Systems

Analytic methods contemplated herein can be implemented by use of computer systems and methods described below and known in the art. Thus, the invention provides computer readable media comprising one or more GAS markers, and optionally other markers. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on one or more GAS markers, and optionally other markers.

A variety of data processor programs and formats can be used to store information on one or more GAS markers and other markers on computer readable medium. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of data processor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining whether a patient has a GAS disease, comprising determining the presence or absence of one or more GAS markers, and optionally other markers, and based on the presence or absence of the one or more GAS markers and optionally other markers, determining a GAS disease, and optionally recommending a procedure or treatment.

In an aspect of the invention a method is provided for detecting a GAS disease using a computer having a processor, memory, display, and input/output devices, the method comprising the steps of:
(a) creating records of one or more GAS markers, and optionally other markers of GAS disease in a sample suspected of containing GAS markers;
(b) providing a database comprising records of data comprising one or more GAS markers, and optionally other markers; and
(c) using a code mechanism for applying queries based upon a desired selection criteria to the data file in the database to produce reports of records of step (a) which provide a match of the desired selection criteria of the database of step (b), the presence of a match being a positive indication that the markers of step (a) have been isolated from a sample of an individual with a GAS disease.

In an aspect of the invention, the computer systems, components, and methods described herein are used to monitor disease or determine the stage of disease.

### Kits

The invention also contemplates kits for carrying out the methods of the invention. Kits may typically comprise two or more components required for performing a diagnostic assay. Components include but are not limited to compounds, reagents, containers, and/or equipment.

The methods described herein may be performed by utilizing pre-packaged diagnostic kits comprising one or more specific GAS marker polynucleotide or antibody described herein, which may be conveniently used, e.g., in clinical settings to screen and diagnose patients and to screen and identify those individuals exhibiting a predisposition to developing a GAS disease.

In an embodiment, a container with a kit comprises a binding agent as described herein. By way of example, the kit may contain antibodies or antibody fragments which bind specifically to epitopes of one or more GAS marker polypeptides and optionally other markers, antibodies against the antibodies labelled with an enzyme; and a substrate for the enzyme. The kit may also contain microtiter plate wells, standards, assay diluent, wash buffer, adhesive plate covers, and/or instructions for carrying out a method of the invention using the kit.

In an aspect of the invention, the kit includes antibodies or fragments of antibodies which bind specifically to an epitope of one or more GAS marker polypeptide and means for detecting binding of the antibodies to their epitopes, either as concentrates (including lyophilized compositions), which may be further diluted prior to use or at the concentration of use, where the vials may include one or more dosages. Where the kits are intended for in vivo use, single dosages may be provided in sterilized containers, having the desired amount and concentration of agents. Containers that provide a formulation for direct use, usually do not require other reagents, as for example, where the kit contains a radiolabelled antibody preparation for in vivo imaging.

A kit may be designed to detect the level of GAS marker polynucleotides in a sample. In an embodiment, the polynucleotides encode one or more GAS marker polynucleotides listed in Tables 3, 4 and 5. Such kits generally comprise at least one oligonucleotide probe or primer, as described herein, that hybridizes to a polynucleotide encoding one or more GAS marker polypeptide. Such an oligonucleotide may be used, for example, within a PCR or hybridization procedure. Additional components that may be present within the kits include a second oligonucleotide and/or a diagnostic reagent or container to facilitate detection of a polynucleotide encoding one or more GAS marker polypeptide.

The invention provides a kit containing a microarray described herein ready for hybridization to target GAS polynucleotide markers, plus software for the analysis of the results. The software to be included with the kit comprises data analysis methods, in particular mathematical routines for marker discovery, including the calculation of correlation coefficients between clinical categories and marker expression. The software may also include mathematical routines for calculating the correlation between sample marker expression and control marker expression, using array-generated fluorescence data, to determine the clinical classification of the sample.

The reagents suitable for applying the screening methods of the invention to evaluate compounds may be packaged into convenient kits described herein providing the necessary materials packaged into suitable containers.

The invention contemplates a kit for assessing the presence of GAS, wherein the kit comprises antibodies specific for one or more GAS markers, or primers or probes for polynucleotides encoding same, and optionally probes, primers or antibodies specific for other markers associated with a GAS disease.

The invention comprises assay kits (e.g., articles of manufacture) for detecting anti-GAS marker polypeptide antibodies or antibody fragments in a sample. A kit comprises one or more GAS marker polypeptides and means for determining binding of the polypeptides to antibodies or antibody fragments in the sample. A kit can comprise a device containing one or more GAS marker polypeptides and instructions for use of the one or more polypeptides e.g., the identification of a GAS disease or GAS infection in a mammal. The kit can also comprise packaging material comprising a label that indicates that the one or more polypeptides of the kit can be used for the identification of a GAS disease or infection. Other components such as buffers, controls, and the like, known to those of ordinary skill in art, can be included in such test kits. The polypeptides, antibodies, assays, and kits of the invention are useful, for example, in the diagnosis of individual cases of GAS disease or infection in a patient, as well as epidemiological studies of GAS outbreaks.

The invention relates to a kit for assessing the suitability of each of a plurality of test compounds for inhibiting a GAS disease in a patient. The kit comprises reagents for assessing one or more GAS markers, and optionally a plurality of test agents or compounds.

### Therapeutic Applications

One or more GAS markers may be targets for immunotherapy. Immunotherapeutic methods include the use of antibody therapy, in vivo vaccines, and ex vivo immunotherapy approaches.

In one aspect, the invention provides one or more antibodies specific for one or more GAS marker polypeptide that may be used systemically to treat a GAS disease associated with the marker. In particular, the GAS disease is strep throat, scarlet fever, impetigo, cellulitis-erysipelas, rheumatic fever, acute glomerular nephritis, endocarditis, or necrotizing fasciitis and one or more GAS marker antibodies may be used systemically to treat such disease.

Thus, the invention provides a method of treating a patient susceptible to, or having a disease that expresses one or more GAS marker polypeptide comprising administering to the patient an effective amount of an antibody that binds specifically to one or more GAS marker polypeptide.

One or more GAS marker antibodies may also be used in a method for selectively inhibiting the growth or, or killing GAS expressing one or more GAS marker comprising reacting one or more GAS marker antibody immunoconjugate or immunotoxin with the cell in an amount sufficient to inhibit the growth of, or kill GAS.

By way of example, unconjugated antibodies to GAS marker polypeptides may be introduced into a patient such that the antibodies bind to GAS expressing GAS marker polypeptides and mediate growth inhibition of such GAS (including the destruction thereof). In addition to unconjugated antibodies to GAS marker polypeptides, one or more GAS marker polypeptide antibodies conjugated to therapeutic agents (e.g. immunoconjugates) may also be used therapeutically to deliver the agent directly to one or more GAS expressing GAS marker polypeptides and thereby destroy the GAS. Examples of such agents include abrin, ricin A, Pseudomonas exotoxin, or diphtheria toxin.

In the practice of a method of the invention, GAS marker polypeptide antibodies capable of inhibiting the growth of GAS expressing GAS marker polypeptides are administered in a therapeutically effective amount to patients with a GAS disease. The invention may provide a specific and effective treatment for a GAS disease. The antibody therapy methods of the invention may be combined with other therapies including antibiotics.

GAS marker polypeptide antibodies useful in treating a GAS disease include those that are capable of initiating a potent immune response against the disease and those that are capable of direct cytotoxicity. In this regard, GAS marker polypeptide antibodies may elicit cell lysis by either complement-mediated or antibody-dependent cell cytotoxicity (ADCC) mechanisms, both of which require an intact Fc portion of the immunoglobulin molecule for interaction with effector cell Fc receptor sites or complement proteins.

GAS marker polypeptide antibodies that exert a direct biological effect on GAS may also be useful in the practice of the invention. Such antibodies may not require the complete immunoglobulin to exert the effect. Potential mechanisms by which such directly cytotoxic antibodies may act include inhibition of cell growth. The mechanism by which a particular antibody exerts an effect may be evaluated using any number of in vitro assays designed to determine ADCC, antibody-dependent macrophage-mediated cytotoxicity (ADMMC), complement-mediated cell lysis, and others known in the art.

The methods of the invention contemplate the administration of single GAS marker antibodies as well as combinations, or “cocktails”, of different individual antibodies such as those recognizing different epitopes of other markers. Such cocktails may have certain advantages inasmuch as they contain antibodies that bind to different epitopes of GAS markers. Such antibodies in combination may exhibit synergistic therapeutic effects. In addition, the administration of one or more GAS marker polypeptide specific antibodies may be combined with other therapeutic agents, including but not limited to antibiotics. The GAS marker specific antibodies may be administered in their “naked” or unconjugated form, or may have therapeutic agents conjugated to them.

The GAS marker polypeptide specific antibodies used in the methods of the invention may be formulated into pharmaceutical compositions comprising a carrier suitable for the desired delivery method. Suitable carriers include any material which when combined with the antibodies retains the function of the antibody and is non-reactive with the subject's immune systems. Examples include any of a number of standard pharmaceutical carriers such as sterile phosphate buffered saline solutions, bacteriostatic water, and the like (see, generally, Remington: The Science and Practice of Pharmacy, 21^{st} Edition. University of the Sciences in Philadelphia (Editor), Mack Publishing Company).

One or more GAS marker polypeptide specific antibody formulations may be administered via any route capable of delivering the antibodies to the disease site. Routes of administration include, but are not limited to, intravenous, intraperitoneal, intramuscular, intradermal, and the like. Preferably, the route of administration is by intravenous injection. Antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration such as intravenous injection (IV), at an effective dose. Dosages will depend upon various factors generally appreciated by those of skill in the art, including the type of disease and the severity, stage of the disease, the binding affinity and half life of the antibodies used, the degree of GAS marker expression in the patient, the extent of GAS markers, the desired steady-state antibody concentration level, frequency of treatment, and the influence of any therapeutic agents used in combination with the treatment method of the invention. Daily doses may range from about 0.1 to 100 mg/kg. Doses in the range of 10-500 mg antibodies per week may be effective and well tolerated, although even higher weekly doses may be appropriate and/or well tolerated. A determining factor in defining the appropriate dose is the amount of a particular antibody necessary to be therapeutically effective in a particular context. Repeated administrations may be required to achieve disease inhibition or regression. Direct administration of one or more GAS marker antibodies is also possible and may have advantages in certain situations.

Patients may be evaluated for serum GAS markers in order to assist in the determination of the most effective dosing regimen and related factors. Assay methods described herein, or similar assays, may be used for quantitating circulating GAS marker levels in patients prior to treatment. Such assays may also be used for monitoring throughout therapy, and may be useful to gauge therapeutic success in combination with evaluating other parameters such as serum levels of GAS markers.

The invention further provides vaccines formulated to contain one or more GAS marker or fragment thereof. In an embodiment, the invention provides a method of vaccinating an individual against one or more GAS marker polypeptide comprising the step of inoculating the individual with the marker or fragment thereof that lacks activity, wherein the inoculation elicits an immune response in the individual thereby vaccinating the individual against the marker.

Viral gene delivery systems may be used to deliver one or more GAS marker polynucleotides. Various viral gene delivery systems which can be used in the practice of this aspect of the invention include, but are not limited to, vaccinia, fowlpox, canarypox, adenovirus, influenza, poliovirus, adeno-associated virus, lentivirus, and sindbus virus (Restifo, 1996, Curr. Opin. Immunol. 8: 658-663). In aspects of the invention, vectors derived from retroviruses, adenovirus, herpes or vaccinia viruses, or from various bacterial plasmids, may be used to deliver polynucleotides encoding GAS marker polypeptides to a targeted site. Methods well known to those skilled in the art may be used to construct recombinant vectors that will express antisense polynucleotides for GAS marker polypeptides. (See, for example, the techniques described in Sambrook et al (supra) and Ausubel et al [5]). Methods for introducing vectors into cells or tissues include those methods discussed herein and which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art. Non-viral delivery systems may also be employed by using naked DNA encoding one or more GAS marker polypeptide or fragment thereof introduced into the patient (e.g., intramuscularly) to induce a response.

Anti-idiotypic GAS marker polypeptide specific antibodies can also be used in therapy as a vaccine for inducing an immune response to GAS that express one or more GAS markers. The generation of anti-idiotypic antibodies is well known in the art and can readily be adapted to generate anti-idiotypic GAS marker polypeptide specific antibodies that mimic an epitope on one or more GAS marker polypeptides (see, for example, Wagner et al., 1997, Hybridoma 16: 33-40; Foon et al., 1995, J Clin Invest 96: 334-342). Such an antibody can be used in anti-idiotypic therapy as presently practiced with other anti-idiotypic antibodies directed against antigens associated with disease.

Genetic immunization methods may be utilized to generate prophylactic or therapeutic humoral and cellular immune responses directed against GAS expressing one or more GAS marker polypeptides. One or more DNA molecules encoding GAS markers, constructs comprising DNA encoding one or more GAS markers/immunogens and appropriate regulatory sequences may be injected directly into muscle or skin of an individual, such that the cells of the muscle or skin take-up the construct and express the encoded GAS markers/immunogens. The GAS markers/immunogens may be expressed as cell surface proteins or be secreted. Expression of one or more GAS markers results in the generation of prophylactic or therapeutic humoral and cellular immunity against a GAS disease. Various prophylactic and therapeutic genetic immunization techniques known in the art may be used.

In another aspect, the invention provides methods for selectively inhibiting GAS expressing GAS marker polypeptide by reacting any one or a combination of the immunoconjugates of the invention with the GAS in an amount sufficient to inhibit the GAS. Amounts include those that are sufficient to kill the GAS or sufficient to inhibit cell growth

One or more GAS markers and fragments thereof may be used in the treatment of a GAS disease in a subject. The GAS markers may be formulated into compositions for administration to subjects suffering from a GAS disease. Therefore, the present invention also relates to a composition comprising one or more GAS markers, or a fragment thereof, and a pharmaceutically acceptable carrier, excipient or diluent. A method for treating or preventing a GAS disease in a subject is also provided comprising administering to a patient in need thereof, one or more GAS markers, or a composition of the invention.

The invention further provides a method of inhibiting a GAS disease in a patient comprising:
(a) obtaining a sample containing GAS markers from the patient;
(b) separately maintaining aliquots of the sample in the presence of a plurality of test agents;
(c) comparing levels of one or more GAS markers in each aliquot;
(d) administering to the patient at least one of the test agents which alters the levels of the GAS markers in the aliquot containing that test agent, relative to the other test agents.

An active therapeutic substance described herein may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance. Solutions of an active compound as a free base or pharmaceutically acceptable salt can be prepared in an appropriate solvent with a suitable surfactant. Dispersions may be prepared in glycerol, liquid polyethylene glycols, and mixtures thereof, or in oils.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985; Remington: The Science and Practice of Pharmacy, 21^{st} Edition. University of the Sciences in Philadelphia (Editor), Mack Publishing Company). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment. The compositions of the invention may be administered concurrently, separately, or sequentially with other therapeutic agents or therapies.

The therapeutic activity of compositions and agents/compounds identified using a method of the invention and may be evaluated in vivo using a suitable animal model.

The following non-limiting examples are illustrative of the present invention:

### EXAMPLE 1

The pathogenesis of invasive GAS infections was investigated using in vivo induced antigen technology (IVIAT) (See Handfield, M et al, 2000. Trends Microbiol. 8:336-339; Handfield, M et al., 2003. Methods Mol. Med. 71:225-242), a technique that relies on antibodies produced during a natural infection. The IVIAT scheme consists of three steps: (i) serum selection and adsorption, (ii) construction of a GAS genomic expression library, and screening of the GAS genomic library with the selected, pooled, absorbed sera.

### Experimental Procedures

The materials and methods used in the studies in this example are set out below.

Bacterial strains, media and growth conditions: To obtain in vitro induced antigens, GAS strains (Table 1) were cultured overnight at 37° C. in Todd-Hewitt (TH) broth (Difco Laboratories, Detriot, Mich.), under either aerobic or microaerobic (5.0% CO₂) conditions and whole cells, cell extracts and spent media were prepared as described below. GAS strains for inoculation of mice were prepared as previously described (1). The E. coli strains utilized for the construction of the GAS genomic library were grown in Luria-Bertani (LB) broth (Difco Laboratories) at 37° C. under aerobic conditions.

In vitro antigen preparation: Equal volumes of each strain (Table 1) of GAS cultures grown to late-log phase were pooled, centrifuged, the spent media removed, and whole cells re-suspended in 1× phosphate-buffered saline (PBS). Cell extracts were prepared from whole cells that were concentrated 10-fold and processed with an FP120 Fastprep Machine (BIO 101, Mississauga, Canada) at a setting of 6.0 for 30 sec, placed on ice for 30 min to allow the beads to settle, and cell extracts removed by aspiration. Denatured cell extracts were obtained by placing cell extracts in a boiling water bath for 10 min. The pooled, cell-free supernatant was freeze-dried using a Benchtop 3.3/Vacu-Freeze Dryer (VirTris Company, Gardiner, N.Y.) and re-suspended in 1×PBS. All antigen preparations were stored at −70° C., for up to 1 month, until ready for use.

Human Sera: Convalescent human sera, collected between 2 to 3 weeks following diagnosis, were selected from 14 of 21 patients with invasive GAS infections, such as NF and STSS (Table 2). Note, that these serum samples were not from the same patients as those from whom the 8 invasive GAS isolates were collected (Table 1). Hence, the strain and serum samples were not paired. Control human sera were obtained from human subjects with no previous history of invasive GAS infection.

Mice sera: Immunocompetent 4 week-old, female, crl:SKH1 (hrhr) hairless mice (Charles River Laboratories, Wilmington, Mass.) were utilized for the invasive subcutaneous infection model of GAS (1). This mouse model was used for generating anti-GAS mouse antibodies for screening the GAS genomic library. Two mice were infected with each of the 8 invasive GAS strains (Table 1) to give a total of 16 infected mice. The immunization protocol included an initial inoculation of 10⁶ colony forming units (CFU), followed by a primary boost (10⁶ CFU for those mice that developed lesions and 10⁸ CFU for those mice that did not develop a lesion) after 2 weeks, and a secondary boost (10³ CFU) after an additional 2 weeks. Sera from 10 non-infected mice were used as controls against sera from GAS-immunized mice. Serum was obtained by cardiac puncture and stored at −70° C. All experimental procedures were in accordance with the principles of the Animal Care Committee of Mount Sinai Hospital, Toronto, Canada.

Indirect ELISA: An indirect ELISA was used for screening the human and mice sera using in vitro-derived GAS antigens (refer to in vitro antigen preparation). Immulon IIHB plates (Dynex Technologies, Chantilly, Va.) were coated overnight at 4° C. with each antigen (whole cells, cell extracts and spent media) which was diluted in freshly prepared carbonate bi-carbonate (C/BC) buffer consisting of 20 mM sodium carbonate (Fisher Scientific, Nepean, Canada) and 50 mM sodium bi-carbonate (BDH Chemicals, Toronto, Canada). The assay procedure described previously was followed (2). The antibody titre was defined as the highest serial dilution of serum at which the OD₄₉₀ was 2 standard deviations above the mean OD₄₉₀ of the negative controls (without primary antibody or without antigen). Antibody titres were converted to logarithmic values (log₂ (x), where x equals the reciprocal of the serum dilution) for calculation of geometric means.

Sera adsorption: Equal volumes of selected invasive patient sera and GAS-immunized mice sera were pooled in a species-specific manner and successively adsorbed with in vitro-derived GAS antigens. In addition, sera from 14 healthy individuals were also pooled and successively adsorbed with in vitro-derived GAS antigens. The successive adsorption steps consisted of 5 times with whole cells, 1 time with cell extracts, 1 time with denatured cell extracts, and 1 time with spent media. Adsorptions were carried out by incubating the pooled sera overnight at 4° C. with antigen-saturated nitrocellulose membranes (Millipore, Bedford, Mass.). After each successive adsorption, the pooled sera were removed and the membrane was washed with 500 μl 1×PBS, which was then added to the pooled sera. To check the efficacy of each adsorption step, a 10 μl aliquot of the serum was removed after each adsorption and an indirect ELISA performed.

Construction of an inducible expression GAS genomic DNA library: Chromosomal DNA from 8 GAS strains (Table 1) was extracted using a cetyltrimethylammonium bromide (CTAB) protocol (5). The library was constructed by partial Sau3AI digestion of the genomic DNA that was ligated into pET30-abc vectors (Novagen Inc., Madison, Wis.), and electroporated into E. coli DH10B non-expression cells (Invitrogen, Ontario, Canada) as described previously (3).

Genomic library screening: An aliquot of the plasmid DNA library in E. coli DH10B non-expression hosts was extracted using the QIAprep Spin Miniprep kit (Qiagen Inc, Ontario, Canada) and transformed into chemically competent E. coli BL21 (DE3) expression host (Novagen). The library was screened by Colony Western blot analysis with pooled adsorbed or unadsorbed human and mouse sera in a species specific-manner as described previously (3) utilizing a Western Blotting Detection Kit (Bio-Rad Laboratories, Hercules, Calif.).

DNA sequencing: DNA sequencing was done with an ABI Prism 377 automatic DNA sequencer by the double-strand dideoxy-chain termination method at the Hospital for Sick Children Sequencing Facility, Toronto, Canada. Sequences were analyzed using the BLAST algorithm of the National Center for Biotechnology Information (NCBI).

### Results

Antigenic determinants upregulated during invasive GAS infections were identified using in vivo antigen technology (IVIAT) [6]. The results are shown in Tables 3, 4 and 5. The markers listed in Tables 3, 4 and 5 are associated with GAS diseases and may be upregulated during invasive GAS infections.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the antibodies, methodologies etc. which are reported therein which might be used in connection with the invention. Nothing herein is to be construed as an admission that the invention is not entitled to antedate such disclosure by virtue of prior invention.

### REFERENCES

1. Betschel, S. D., Borgia, S. M., Barg, N. L., Low, D. E., and de Azavedo, J. C. 1998. Reduced virulence of group A streptococcal Tn916 mutants that do not produce streptolysin S. Infect. Immun. 66: 1671-1679.
2. Crowther, J. R. 2001. The ELISA Guidebook. Humana Press, Inc., USA, pp. 45-82.
3. Kim, Y. R., Lee, S. E., Kim, C. M., Kim, S. Y., Shin, E, K., Shin, D. H., Chung, S. S., Choy, H. E., Progulske-Fox, A., Hillman, J. D., Handfield, M., and Rhee, J. H. 2003. Characterization and pathogenic significance of Vibrio vulnificus antigens preferentially expressed in septicemic patients. Infect. Immun. 71: 5461-5471.
4. Musser, J. M., Kapur, V., Kanjilal, S., Shah, U., Musher, D. M., Barg, N. L., Johnston, K. H., Schlievert, P. M., Henrichsen, J., Gerlach, D., Rakita, R. M., Tanna, A., Cookson, B. D., and Huang, J. C. 1993. Geographic and temporal distribution and molecular characterization of two highly pathogenic clones of Streptococcus pyogenes expressing allelic variants of pyrogenic exotoxin A (scarlet fever toxin). J. Infect. Dis. 167: 337-346.
5. Wilson, K. 1994. Preparation of genomic DNA from bacteria. In Current Protocols in Molecular Biology, Vol. 1, Ausubel, F. M., Brent, R., Kingston, E., Moore, D. D., Seidman, J. G., Smith, J. A., and Struhl, K. (eds.). John Wiley & Sons, NY, pp. 2.4.1-2.4.5.
6. Salim, K., D. G. Cvitkovitch, P. Chang, D. J. Bast, M. Handfield, J. D. Hillman and de Azavedo, J. C. 2005. Identification of Group A Streptococcus Antigenic Determinants Upregulated In Vivo. Infection Immunity 73(9): 6026-6038.

TABLE 1
Eight invasive clinical strains of Group A Streptococcus used
for the construction of the GAS genomic library and for deriving
the in vitro antigens used to adsorb sera from patients with
invasive disease, GAS-immunized mice, and healthy individuals.

| M/T Type Of Gas Strain | Source Of Gas Isolate | Case Definition |
|---|---|---|
| M1/T1 | Blood | Invasive |
| M1/T1 | Throat | Invasive |
| M3/T3 | Blood | STSS |
| M4/T4 | Blood | Invasive |
| M6/Tnontyp. | Blood | STSS |
| M11/T11 | Blood | Invasive |
| M12/T12 | Blood | Invasive |
| M28/T11/28 | Blood | Invasive |

STSS = Streptococcal Toxic Shock Syndrome

ABLE 2
A list of sera from patients with invasive GAS disease
used for screening the GAS genomic library.

| Sera | | Source of GAS | M type of GAS |
|---|---|---|---|
| # | Diagnosis | isolate | isolate |
| 1 | NF & STSS | Abscess | M1 |
| 2 | Cellulitis | Abscess | M non-typeable |
| 3 | Cellulitis | Blood & abscess | M1 |
| 4 | Cellulitis & arthritis | Blood | M12 |
| 5 | NF & STSS | Blood & abscess | M75 |
| 6 | NF | Tissue | N/A |
| 7 | Cellulitis | Blood & abscess | M28 |
| 8 | Arthritis | Throat/aspirate | M3 |
| 9 | NF & STSS | Abscess | M1 |
| 10 | Necrotizing myositis & STSS | Blood | N/A |
| 11 | Peritonitis | Blood | N/A |
| 12 | Pneumonia | Blood | M1 |
| 13 | NF | Abscess | M4 |
| 14 | Cellulitis | Abscess | M1 |

NF = Necrotizing Fasciitis
STSS = Streptococcal Toxic Shock Syndrome

TABLE 3
List of GAS Markers

| | | Blast search - GAS |
|---|---|---|
| Sera indicating | Insert size | Best hit - organism & gene |
| positive | (bp) | Possible role |
| Human & Mouse | 500 | prfB (spy 0643/spy M18 0705) |
| | | peptide chain release factor in B. subtilis |
| | | putative peptide chain release factor 2 |
| | | ftsE (spy 0644/spyM18 0706-230aa |
| | | ftsE gene in B. subtilis |
| | | putative cell division ATP-binding protein |
| | | (spy 1149) |
| | | hypothetical ABC transporter in Thermatoga maritime |
| | | putative ABC transporter |
| Human & Mouse | 2000 | Hypothetical phage protein |
| | | (spyM18 1298, spyM18 1299, spyM3 0966, spyM3 0967, & |
| | | spyM3 1257) |
| Human | 119 | 16S-23S intergenic spacer |
| Human & Mouse | 415 | dnaQ (spy 1864/spyM18 1928); 208aa/195aa |
| | | Putative DNA pol. III (epsilon subunit) |
| | | (spy 1865/spyM18 1929); 193aa/176aa |
| | | unknown protein in L. lactis |
| | | unknown function |
| | | epf (spy 0737); |
| | | extracellular matrix binding protein in Abiotrophia defective |
| | | putative extracellular matrix binding protein |
| | | rpoE (spy 1895/spyM18 1960); 191aa |
| | | putative DNA-directed RNA pol. (delta subunit) |
| Human & Mouse | 1500 | htsA/siaA |
| | | (spy 1795/spyM3 1560/spyM18 1867) |
| | | ferrichrome binding protein |
| Human | 2000 | papS (spy 0866) |
| | | putative polyA polymerase (M1, M3, & M18) |
| Mouse | 281 | nox (spy 1150/spyM18 1110/JRS4GAS strain); 456aa; |
| | | 246aa in JRS |
| | | NADH oxidase (water forming) in S. mutans |
| | | NADH oxidase |
| Human & Mouse | 184 | amyA (spy 1302); 711aa |
| | | cgtase from Thermoanearo thermosulfurigens Eml in |
| | | complex with a maltohexaose inhibitor |
| | | putative cyclomaltodextrin glucanotransferase |
| Human & Mouse | 152 | (spy 1733/spyM18 1741); 424aa/427aa |
| | | attenuator for lytABC and lytR expression in B. halodurans |
| | | putative transcription regulator |
| Human | 196 | (spy 0430/spyM18 0477); |
| | | fibronectin binding protein SFS in S. equi |
| | | hypothetical protein |
| - | 195 | dnaE (spy 1284/spyM18 1232); 1036aa |
| | | DNA pol. III alpha subunit in B. subtilis |
| | | DNA pol. III alpha subunit in B. subtilis |
| Human & Mouse | 76 | Insert corresponds to spacer between genes below |
| | | clpP (spy 0395/spyM18 0446) |
| | | ATP-dependent CLP protease proteolytic subunit |
| | | (endopeptidase CLP) in S. salivarius |
| | | Putative ATP-dependent protease proteolytic subunit |
| | | (spy 0397/spyM18 0447) |
| | | conserved hypothetical protein ylb in B. subtilis |
| | | conserved hypothetical protein |
| Human & Mouse | 355 | nifS (spy 1122) |
| | | iron-sulfur cofactor synthesis protein yrvO in B. subtilis; |
| | | similar to NifS homolog in B. subtilis |
| | | putative iron-sulfur cofactor synthesis protein |
| | | (spy 1121/spyM18 1152); 115aa |
| | | no blast hits |
| | | unknown function |
| Mouse | 833 | (spy 2031/spyM18 2089); 224aa |
| | | ABC transporter in B. halodurans |
| | | Putative ABC transporter |
| | | Spy 2032/spyM18 2090); 422aa |
| | | Conserved hypothetical protein yrvP in B. subtilis |
| | | Putative ATP-binding cassette transporter-like protein |
| Human & Mouse | 3000 | Spy 0630 & spy 0631 |
| | | Putative PTS dependent N-acetyl-galactosamine IIC |
| | | component & Putative PTS dependent N-acetyl- |
| | | galactosamine IIB component |
| | | SpyM3 1326 |
| | | Conserved hypothetical protein - phage associated (phage |
| | | 315.5) |
| | | purD (Spy 0032) |
| | | phosphoribosylamine glycine ligase |
| | | Spy 1784 |
| | | Putative ABC transporter (ATP-binding protein) |
| Human & Mouse | 157 | Spy 0319 |
| | | atmB in S. mutans |
| | | putative lipoprotein |
| Human Mouse | 113 | Spy 1233 |
| | | coaA, putative pantothenate kinase |
| Human & Mouse | 1733 | spyM3 1096 |
| | | putative N-acetylmuramoyle-L-alanine amidase (lysine |
| | | phage-associated) (S. pyogenes phage 315.3) |
| Human & Mouse | 302 | Spy 1355 & spy 1356 |
| | | Conserved hypothetical protein & putative acetyl transferase |
| Human & Mouse | 2000 | Spy 1961, spy 2060, & spy 2063 |
| | | Putative DNA pol. III alpha subunit (polC), conserved |
| | | hypothetical protein, & putative translation initiation |
| | | inhibitor (tdcF) |
| Human & Mouse | 584 | Spy 0777 |
| | | rexA |
| Human & Mouse | 71 | Spy 1649 |
| | | pbp1A |
| Human & Mouse | 1500 | Spy 1198 |
| | | Putative repressor protein |
| Human & Mouse | 267 | Spy 1733 & spy 1674 |
| | | Putative transcription regulator (lytR) & putative ABC |
| | | transporter |
| Human & Mouse | 152 | Spy 1105 |
| | | Putative spermidine/putrecine ABC transporter (potD) |

TABLE 4
GAS Markers

| Marker # | Gene and Spy# | Sequence ID No. |
|---|---|---|
| 1 | purD (Spy 0032) | 1, 2 |
| 2 | atmB (Spy 0319) | 3, 4 |
| 3 | clpP (Spy 0395) | 5, 6 |
| 4 | Spy 0397 | 7, 8 |
| 5 | Spy 0430 | 9, 10 |
| 6 | Spy 0630 | 11, 12 |
| 7 | Spy 0631 | 13, 14 |
| 8 | prfB (Spy 0643) | 15, 16 |
| 9 | ftsE (Spy 0644) | 17, 18 |
| 10 | epf (Spy 0737) | 19, 20 |
| 11 | rexA (Spy 0777) | 21, 22 |
| 12 | papS (Spy 0866) | 23, 24 |
| 13 | Spy 1096 | 25, 26 |
| 14 | potD (Spy 1105) | 27, 28 |
| 15 | Spy 1121 | 29, 30 |
| 16 | nifS (Spy 1122) | 31, 32 |
| 17 | Spy 1149 | 33, 34 |
| 18 | nox (Spy 1150) | 35, 36 |
| 19 | Spy 1198 | 37, 38 |
| 20 | coaA (Spy 1233) | 39, 40 |
| 21 | dnaE (Spy 1284) | 41, 42 |
| 22 | amyA (Spy 1302) | 43, 44 |
| 23 | Spy 1355 | 45, 46 |
| 24 | Spy 1356 | 47, 48 |
| 25 | pbp1A (Spy 1649) | 49, 50 |
| 26 | Spy 1674 | 51, 52 |
| 27 | lyt (Spy 1733) | 53, 54 |
| 28 | Spy 1784 | 55, 56 |
| 29 | siaA/htsA (Spy 1795) | 57, 58 |
| 30 | dnaQ (Spy 1864) | 59, 60 |
| 31 | Spy 1865 | 61, 62 |
| 32 | rpoE (Spy 1895) | 63, 64 |
| 33 | Spy 1921 | 65, 66 |
| 34 | Spy 2031 | 67, 68 |
| 35 | Spy 2032 | 69, 70 |
| 36 | tdcF (Spy 2060) | 71, 72 |
| 37 | Spy 2063 | 73, 74 |
| 38 | SpyM3 1257 | 75, 76 |
| 39 | SpyM3 1326 | 77, 78 |
| 40 | SpyM18 1298 | 79, 80 |
| 41 | SpyM18 1299 | 81, 82 |
| 42 | Spy 1961 | 83, 84, 85 |

TABLE 5

| | | | |
|---|---|---|---|
| Serotype | Strain | Locus tag | GeneID |

Gene name = phosphoribosylamine--glycine ligase (purD);
Locus tag = Spy_0032; GeneID = 900390;
Reference sequence from = Streptococcus pyogenes
M1GAS (strain SF370, serotype M1); SEQ I.D. Nos. 1 and 2

| | | | |
|---|---|---|---|
| M1 | MGAS5005 | MGAS5005_Spy_0029 | 3572913 |
| M1 | S. pyogenes SSI-1 | SPs0027 | 1066282 |
| M28 | MGAS6180 | M28_0029 | 3573098 |
| M2 | MGAS10270 | MGAS10270_Spy0030 | 4063297 |
| M6 | MGAS10394 | M6_Spy0078 | 2949058 |
| M12 | MGAS9429 | MGAS9429_Spy0029 | 4060501 |
| M3 | MGAS315 | SpyM3_0026 | 1008340 |
| M4 | MGAS10750 | MGAS10750_Spy0030 | 4067237 |
| M5 | Manfredo | SpyM50029 | 4962783 |
| M49 | M49 591 | SpyoM01000109 | n/a |
| M18 | MGAS8232 | SpyM18_0032 | 995239 |
| M12 | MGAS2096 | MGAS2096_0030 | 4065258 |

Gene name = hypothetical protein; Locus tag = Spy_0319;
GeneID = 900605 Reference sequence from =
Streptococcus pyogenes M1GAS (strain SF370,
serotype M1); SEQ I.D. Nos. 3 and 4

| | | | |
|---|---|---|---|
| M1 | MGAS5005 | MGAS5005_Spy_0271 | 3572639 |
| M1 | S. pyogenes SSI-1 | SPs1626 | 1066435 |
| M28 | MGAS6180 | M28_0263 | 3574645 |
| M2 | MGAS10270 | MGAS10270_Spy0268 | 4063807 |
| M6 | MGAS10394 | M6_Spy0299 | 2942436 |
| M12 | MGAS9429 | MGAS9429_Spy0270 | 4060766 |
| M3 | MGAS315 | SpyM3_0233 | 1008547 |
| M4 | MGAS10750 | MGAS10750_Spy0266 | 4066979 |
| M5 | Manfredo | SpyM51584 | 4963797 |
| M18 | MGAS8232 | SpyM18_0314 | 994798 |
| M12 | MGAS2096 | MGAS2096_0289 | 4064734 |

Gene name = clpP ATP-dependent Clp protease proteolytic
subunit (clpP); Locus tag = Spy_0395; GeneID = 900660;
Reference sequence from = Streptococcus pyogenes
M1GAS (strain SF370, serotype M1); SEQ I.D. Nos. 5 and 6

| | | | |
|---|---|---|---|
| M1 | MGAS5005 | MGAS5005_Spy_0328 | 3572579 |
| M1 | S. pyogenes SSI-1 | SPs1572 | 1066487 |
| M28 | MGAS6180 | M28_0317 | 3574680 |
| M2 | MGAS10270 | MGAS10270_Spy0324 | 4063843 |
| M6 | MGAS10394 | M6_Spy0354 | 2942035 |
| M12 | MGAS9429 | MGAS9429_Spy0328 | 4060785 |
| M3 | MGAS315 | SpyM3_0287 | 1008601 |
| M4 | MGAS10750 | MGAS10750_Spy0324 | 4066732 |
| M5 | Manfredo | SpyM51530 | 4963735 |
| M49 | M49 591 | SpyoM01000726 | n/a |
| M18 | MGAS8232 | SpyM18_0446 | 993567 |
| M12 | MGAS2096 | MGAS2096_0347 | 4065059 |

Spy_0397 Gene name = Hypothetical protein; Locus
tag = Spy_0397; GeneID = 900661; Reference
sequence from = Streptococcus pyogenes M1GAS
(strain SF370, serotype M1); SEQ I.D. Nos. 7 and 8

| | | | |
|---|---|---|---|
| M1 | MGAS5005 | MGAS5005_Spy_0329 | 3572580 |
| M1 | S. pyogenes SSI-1 | SPs1571 | 1065198 |
| M28 | MGAS6180 | M28_0318 | 3574681 |
| M2 | MGAS10270 | MGAS10270_Spy0325 | 4063844 |
| M6 | MGAS10394 | M6_Spy0355 | 2942147 |
| M12 | MGAS9429 | MGAS9429_Spy0329 | 4060786 |
| M3 | MGAS315 | SpyM3_0288 | 1008602 |
| M4 | MGAS10750 | MGAS10750_Spy0325 | 4066733 |
| M5 | Manfredo | SpyM51529 | 4964573 |
| M18 | MGAS8232 | SpyM18_0447 | 995255 |
| M49 | M49 591 | SpyoM01000725 | n/a |

Gene name = Hypothetical protein; Locus tag = Spy_0430;
GeneID = 900679 Reference sequence from =
Streptococcus pyogenes M1GAS (strain SF370,
serotype M1); SEQ I.D. Nos. 9 and 10

| | | | |
|---|---|---|---|
| M1 | MGAS5005 | MGAS5005_Spy_0352 | 3572563 |

Gene name = PTS dependent N-acetyl-galactosamine
IIC component (agaW) Locus tag = Spy_0630;
GeneID = 900829; Reference sequence from =
Streptococcus pyogenes M1GAS (strain SF370,
serotype M1); SEQ I.D. Nos. 11 and 12

| | | | |
|---|---|---|---|
| M1 | MGAS5005 | MGAS5005_Spy_0520 | 3572380 |
| M1 | S. pyogenes SSI-1 | SPs1411 | 1066591 |
| M28 | MGAS6180 | M28_0499 | 3573372 |
| M2 | MGAS10270 | MGAS10270_Spy0515 | 4063897 |
| M6 | MGAS10394 | M6_Spy0541 | 2941679 |
| M12 | MGAS9429 | MGAS9429_Spy0511 | 4062048 |
| M3 | MGAS315 | SpyM3_0444 | 1008758 |
| M4 | MGAS10750 | MGAS10750_Spy0539 | 4068022 |
| M5 | Manfredo | SpyM51343 | 4963991 |
| M18 | MGAS8232 | SpyM18_0695 | 994449 |
| M49 | M49 591 | SpyoM01000828 | n/a |
| M12 | MGAS2096 | MGAS2096_Spy0532 | 4065411 |

Gene name = PTS dependent N-acetyl-galactosamine IIB
component (agaV) Locus tag = Spy_0631;
GeneID = 900830; Reference sequence from =
Streptococcus pyogenes M1GAS (strain SF370,
serotype M1); SEQ I.D. Nos 13 and 14

| | | | |
|---|---|---|---|
| M1 | MGAS5005 | MGAS5005_Spy_0521 | 3572381 |
| M1 | S. pyogenes SSI-1 | SPs1410 | 1065268 |
| M28 | MGAS6180 | M28_0500 | 3573373 |
| M2 | MGAS10270 | MGAS10270_Spy0516 | 4063408 |
| M6 | MGAS10394 | M6_Spy0542 | 2941680 |
| M12 | MGAS9429 | MGAS9429_Spy0512 | 4060599 |
| M3 | MGAS315 | SpyM3_0445 | 1008759 |
| M4 | MGAS10750 | MGAS10750_Spy0540 | 4068023 |
| M5 | Manfredo | SpyM51342 | 4964655 |
| M18 | MGAS8232 | SpyM18_0696 | 993480 |
| M49 | M49 591 | SpyoM01000829 | n/a |
| M12 | MGAS2096 | MGAS2096_Spy0533 | 4065412 |

Gene name = peptide chain release factor 2 (prfB); Locus tag =
Spy_0643 GeneID = 900839; Reference sequence from =
Streptococcus pyogenes M1GAS (strain SF370, serotype M1);
SEQ I.D. Nos. 15 and 16

| | | | |
|---|---|---|---|
| M1 | MGAS5005 | MGAS5005_Spy_0530 | 3572390 |
| M1 | S. pyogenes SSI-1 | SPs1402 | 1066597 |
| M28 | MGAS6180 | M28_0509 | 3573382 |
| M2 | MGAS10270 | MGAS10270_Spy0525 | 4063417 |
| M6 | MGAS10394 | M6_Spy0551 | 2940713 |
| M12 | MGAS9429 | MGAS9429_Spy0521 | 4060608 |
| M3 | MGAS315 | SpyM3_0454 | 1008768 |
| M4 | MGAS10750 | MGAS10750_Spy0549 | 4068032 |
| M5 | Manfredo | SpyM51333 | 4964163 |
| M18 | MGAS8232 | prfB (gene name) | 994061 |
| M49 | M49 591 | SpyoM01000670 | n/a |
| M12 | MGAS2096 | MGAS2096_Spy0542 | 4066242 |

Gene name = cell-division ATP-binding protein (ftsE); Locus tag =
Spy_0644 GeneID = 900840; Reference sequence
from = Streptococcus pyogenes M1GAS
(strain SF370, serotype M1); SEQ I.D. No. 17 and 18

| | | | |
|---|---|---|---|
| M1 | MGAS5005 | MGAS5005_Spy_0531 | 3572391 |
| M1 | S. pyogenes SSI-1 | SPs1401 | 1065292 |
| M28 | MGAS6180 | M28_0510 | 3573383 |
| M2 | MGAS10270 | MGAS10270_Spy0526 | 4063418 |
| M6 | MGAS10394 | M6_Spy0552 | 2940714 |
| M12 | MGAS9429 | MGAS9429_Spy0522 | 4060609 |
| M3 | MGAS315 | SpyM3_0455 | 1008769 |
| M4 | MGAS10750 | MGAS10750_Spy0550 | 4068033 |
| M5 | Manfredo | SpyM51332 | 4964167 |
| M18 | MGAS8232 | SpyM18_0706 | 995132 |
| M12 | MGAS2096 | MGAS2096_Spy0543 | 4066243 |

Gene name = extracellular matrix binding protein (epf); Locus
tag = Spy_0737 GeneID = 900919; Reference
sequence from = Streptococcus pyogenes M1GAS
(strain SF370, serotype M1); SEQ I.D. Nos. 19 and 20

| | | | |
|---|---|---|---|
| M1 | MGAS5005 | MGAS5005_Spy_0561 | 3572346 |
| M28 | MGAS6180 | M28_0539 | 3574749 |
| M12 | MGAS9429 | MGAS9429_Spy0613 | 4062181 |
| M4 | MGAS10750 | MGAS10750_Spy0643 | 4067933 |
| M49 | M49 591 | SpyoM01000212 | n/a |
| M12 | MGAS2096 | MGAS2096_Spy0622 | 4066162 |

Gene name = ATP-dependent exonuclease, subunit A (rexA);
Locus tag = Spy_0777; GeneID = 900953;
Reference sequence from = Streptococcus pyogenes
M1GAS (strain SF370, serotype M1); SEQ I.D. Nos. 21 and 22

| | | | |
|---|---|---|---|
| M1 | MGAS5005 | MGAS5005_Spy_0595 | 3572302 |
| M1 | S. pyogenes SSI-1 | SPs1340 | 1065346 |
| M28 | MGAS6180 | M28_0574 | 3574784 |
| M2 | MGAS10270 | MGAS10270_Spy0650 | 4063145 |
| M6 | MGAS10394 | M6_Spy0612 | 2942261 |
| M12 | MGAS9429 | MGAS9429_Spy0649 | 4062412 |
| M3 | MGAS315 | SpyM3_0514 | 1008828 |
| M4 | MGAS10750 | MGAS10750_Spy0680 | 4067893 |
| M5 | Manfredo | SpyM51212 | 4963549 |
| M18 | MGAS8232 | SpyM18_0836 | 994806 |
| M12 | MGAS2096 | MGAS2096_Spy0658 | 4064540 |

Gene name = poly(A) polymerase (papS); Locus tag =
Spy_0866; GeneID = 901027; Reference sequence
from = Streptococcus pyogenes M1GAS (strain
SF370, serotype M1); SEQ I.D. Nos. 23 and 24

| | | | |
|---|---|---|---|
| M1 | MGAS5005 | MGAS5005_Spy_0673 | 3572224 |
| M1 | S. pyogenes SSI-1 | SPs1266 | 1065346 |
| M28 | MGAS6180 | M28_0653 | 3574823 |
| M2 | MGAS10270 | MGAS10270_Spy0731 | 4062468 |
| M6 | MGAS10394 | M6_Spy0691 | 2942456 |
| M12 | MGAS9429 | MGAS9429_Spy0728 | 4061993 |
| M3 | MGAS315 | SpyM3_0587 | 1008901 |
| M4 | MGAS10750 | MGAS10750_Spy0764 | 4067821 |
| M5 | Manfredo | SpyM51134 | 4963504 |
| M18 | MGAS8232 | SpyM18_0927 | 994047 |
| M49 | M49 591 | SpyoM01000092 | n/a |
| M12 | MGAS2096 | MGAS2096_Spy0744 | 4066108 |

Gene name = putative folyl-polyglumate synthetase (folC.1);
Locus tag = Spy_1096; GeneID = 901217; Reference
sequence from = Streptococcus pyogenes M1GAS
(strain SF370, serotype M1); Sequence I.D. Nos. 25 and 26

| | | | |
|---|---|---|---|
| M1 | MGAS5005 | MGAS5005_Spy_0820 | 3572107 |
| M1 | S. pyogenes SSI-1 | SPs0958 | 1066227 |
| M28 | MGAS6180 | M28_0797 | 3573546 |
| M2 | MGAS10270 | MGAS10270_Spy0936 | 4063937 |
| M6 | MGAS10394 | M6_Spy0818 | 2942407 |
| M12 | MGAS9429 | MGAS9429_Spy0939 | 4062278 |
| M3 | MGAS315 | SpyM3_0758 | 1009072 |
| M4 | MGAS10750 | MGAS10750_Spy0971 | 4067069 |
| M5 | Manfredo | SpyM50968 | 4964153 |
| M18 | MGAS8232 | SpyM18_1058 | 993530 |
| M49 | M49 591 | SpyoM01000299 | n/a |
| M12 | MGAS2096 | MGAS2096_Spy0894 | 4065935 |

Gene name = putative spermidine/putrescine ABC
transporter (periplasmic transport protein (potD); Locus
tag = Spy_1105; GeneID = 901226; Reference
sequence from = Streptococcus pyogenes M1GAS
(strain SF370, serotype M1) SEQ I.D. Nos. 27 and 28

| | | | |
|---|---|---|---|
| M1 | MGAS5005 | MGAS5005_Spy_0829 | 3572077 |
| M1 | S. pyogenes SSI-1 | SPs0967 | 1066226 |
| M28 | MGAS6180 | M28_0806 | 3574881 |
| M2 | MGAS10270 | MGAS10270_Spy0945 | 4063946 |
| M6 | MGAS10394 | M6_Spy0827 | 2941036 |
| M12 | MGAS9429 | MGAS9429_Spy0948 | 4062287 |
| M3 | MGAS315 | SpyM3_0767 | 1009081 |
| M4 | MGAS10750 | MGAS10750_Spy0908 | 4067098 |
| M5 | Manfredo | SpyM50959 | 4963387 |
| M18 | MGAS8232 | SpyM18_1067 | 994023 |
| M49 | M49 591 | SpyoM01000290 | n/a |
| M12 | MGAS2096 | MGAS2096_Spy0903 | 4065887 |

Gene name = hypothetical protein; Locus tag =
Spy_1121; GeneID = 901239 Reference sequence
from = Streptococcus pyogenes M1GAS (strain SF370,
serotype M1); SEQ I.D. Nos. 29 and 30

| | | | |
|---|---|---|---|
| M1 | MGAS5005 | MGAS5005_Spy_0843 | 3572052 |
| M1 | S. pyogenes SSI-1 | SPs0981 | 1066277 |
| M28 | MGAS6180 | M28_0819 | 3574894 |
| M2 | MGAS10270 | MGAS10270_Spy0959 | 4064057 |
| M6 | MGAS10394 | M6_Spy0841 | 2941140 |
| M12 | MGAS9429 | MGAS9429_Spy0962 | 4060549 |
| M3 | MGAS315 | SpyM3_0780 | 1009094 |
| M4 | MGAS10750 | MGAS10750_Spy0994 | 4067112 |
| M5 | Manfredo | SpyM50945 | 4963376 |
| M18 | MGAS8232 | SpyM18_1082 | 994828 |
| M49 | M49 591 | SpyoM01000612 | n/a |
| M12 | MGAS2096 | MGAS2096_Spy0918 | 4065902 |

Gene name = putative iron-sulfur cofactor synthesis protein;
Locus tag = Spy_1122 GeneID = 901240; Reference
sequence from = Streptococcus pyogenes M1GAS
(strain SF370, serotype M1); SEQ I.D. Nos. 31 and 32

| | | | |
|---|---|---|---|
| M1 | MGAS5005 | MGAS5005_Spy_0844 | 3572053 |
| M1 | S. pyogenes SSI-1 | SPs0982 | 1066256 |
| M28 | MGAS6180 | M28_0820 | 3574895 |
| M2 | MGAS10270 | MGAS10270_Spy0960 | 4064058 |
| M6 | MGAS10394 | M6_Spy0842 | 2942248 |
| M12 | MGAS9429 | MGAS9429_Spy0963 | 4060550 |
| M3 | MGAS315 | SpyM3_0781 | 1009095 |
| M4 | MGAS10750 | MGAS10750_Spy0995 | 4067113 |
| M5 | Manfredo | SpyM50944 | 4964387 |
| M18 | MGAS8232 | SpyM18_1083 | 994464 |
| M49 | M49 591 | SpyoM01000611 | n/a |
| M12 | MGAS2096 | MGAS2096_Spy0919 | 4065903 |

Gene name = putative ABC transporter; Locus tag =
Spy_1149; GeneID = 901265 Reference
sequence from = Streptococcus pyogenes M1GAS
(strain SF370, serotype M1); SEQ I.D. Nos. 33 and 34

| | | | |
|---|---|---|---|
| M1 | MGAS5005 | MGAS5005_Spy_0871 | 3572041 |
| M1 | S. pyogenes SSI-1 | SPs1006 | 1066323 |
| M28 | MGAS6180 | M28_0845 | 3573594 |
| M2 | MGAS10270 | MGAS10270_Spy0985 | 4064445 |
| M6 | MGAS10394 | M6_Spy0867 | 2941961 |
| M12 | MGAS9429 | MGAS9429_Spy0989 | 4060826 |
| M3 | MGAS315 | SpyM3_0807 | 1009121 |
| M4 | MGAS10750 | MGAS10750_Spy1020 | 4067320 |
| M5 | Manfredo | SpyM50919 | 4964318 |
| M18 | MGAS8232 | SpyM18_1109 | 993707 |
| M49 | M49 591 | SpyoM01000341 | n/a |
| M12 | MGAS2096 | MGAS2096_Spy0945 | 4065851 |

Gene name = NADH oxidase (nox); Locus tag =
Spy_1150; GeneID = 901266 Reference
sequence from = Streptococcus pyogenes M1GAS
(strain SF370, serotype M1); SEQ I.D. Nos. 35 and 36

| | | | |
|---|---|---|---|
| M1 | MGAS5005 | MGAS5005_Spy_0872 | 3572042 |
| M1 | S. pyogenes SSI-1 | SPs1007 | 1066318 |
| M28 | MGAS6180 | M28_0846 | 3573595 |
| M2 | MGAS10270 | MGAS10270_Spy0986 | 4064446 |
| M6 | MGAS10394 | M6_Spy0868 | 2941962 |
| M12 | MGAS9429 | MGAS9429_Spy0990 | 4060827 |
| M3 | MGAS315 | SpyM3_0808 | 1009122 |
| M4 | MGAS10750 | MGAS10750_Spy1021 | 4067321 |
| M5 | Manfredo | SpyM50918 | 4964253 |
| M18 | MGAS8232 | SpyM18_1110 | 994776 |
| M49 | M49 591 | SpyoM01000340 | n/a |
| M12 | MGAS2096 | MGAS2096_Spy0946 | 4065852 |

Gene name = putative repressor protein; Locus tag =
Spy_1198; GeneID = 901304 Reference
sequence from = Streptococcus pyogenes M1GAS
(strain SF370, serotype M1); SEQ I.D. Nos. 37 and 38

| | | | |
|---|---|---|---|
| M1 | MGAS5005 | MGAS5005_Spy_0914 | 3572006 |
| M1 | S. pyogenes SSI-1 | SPs1040 | 1065479 |
| M28 | MGAS6180 | M28_0886 | 3573577 |
| M2 | MGAS10270 | MGAS10270_Spy1028 | 4063272 |
| M6 | MGAS10394 | M6_Spy0903 | 2942214 |
| M12 | MGAS9429 | MGAS9429_Spy1016 | 4061974 |
| M3 | MGAS315 | SpyM3_0840 | 1009154 |
| M4 | MGAS10750 | MGAS10750_Spy1063 | 4066566 |
| M5 | Manfredo | SpyM50884 | 4963336 |
| M18 | MGAS8232 | SpyM18_1150 | 994630 |
| M49 | M49 591 | SpyoM01001185 | n/a |
| M12 | MGAS2096 | MGAS2096_Spy0973 | 4065840 |

Gene name = pantothenate kinase (coaA); Locus tag =
Spy_1233; GeneID = 901335; Reference sequence
from = Streptococcus pyogenes M1GAS (strain
SF370, serotype M1); SEQ I.D. Nos. 39 and 40

| | | | |
|---|---|---|---|
| M1 | MGAS5005 | MGAS5005_Spy_0945 | 3571959 |
| M1 | S. pyogenes SSI-1 | SPs1071 | 1065683 |
| M28 | MGAS6180 | M28_0917 | 3573647 |
| M2 | MGAS10270 | MGAS10270_Spy1059 | 4063674 |
| M6 | MGAS10394 | M6_Spy0934 | 2941285 |
| M12 | MGAS9429 | MGAS9429_Spy1048 | 4061315 |
| M3 | MGAS315 | SpyM3_0871 | 1009186 |
| M4 | MGAS10750 | MGAS10750_Spy1094 | 4068176 |
| M5 | Manfredo | SpyM50853 | 4964042 |
| M18 | MGAS8232 | SpyM18_1183 | 994086 |
| M49 | M49 591 | SpyoM01001329 | n/a |
| M12 | MGAS2096 | MGAS2096_Spy1004 | 4065023 |

Gene name = DNA polymerase III subunit alpha (dnaE);
Locus tag = Spy_1284 GeneID = 901377;
Reference sequence from = Streptococcus pyogenes
M1GAS (strain SF370 serotype M1); SEQ I.D. Nos. 41 and 42

| | | | |
|---|---|---|---|
| M1 | MGAS5005 | MGAS5005_Spy_0990 | 3571926 |
| M1 | S. pyogenes SSI-1 | SPs1113 | 1065631 |
| M28 | MGAS6180 | M28_0962 | 3573711 |
| M2 | MGAS10270 | MGAS10270_Spy1104 | 4064427 |
| M6 | MGAS10394 | M6_Spy0977 | 2942246 |
| M12 | MGAS9429 | MGAS9429_Spy1094 | 4060986 |
| M3 | MGAS315 | SpyM3_0914 | 1009229 |
| M4 | MGAS10750 | MGAS10750_Spy1140 | 4067152 |
| M5 | Manfredo | SpyM50811 | 4963291 |
| M18 | MGAS8232 | SpyM18_1232 | 994375 |
| M49 | M49 591 | SpyoM01001427 | n/a |
| M12 | MGAS2096 | MGAS2096_Spy1050 | 4065274 |

Gene name = putative cyclomaltodextrin glucanotransferase
(amyA); Locus tag = Spy_1302; GeneID =
901394; Reference sequence from = Streptococcus pyogenes
M1GAS (strain SF370, serotype M1); SEQ I.D. Nos. 43 and 44

| | | | |
|---|---|---|---|
| M1 | MGAS5005 | MGAS5005_Spy_1065 | 3571845 |
| M28 | MGAS6180 | M28_1046 | 3573776 |
| M2 | MGAS10270 | MGAS10270_Spy1121 | 4062598 |
| M4 | MGAS10750 | MGAS10750_Spy1158 | 4066697 |
| M4 | MGAS10750 | MGAS10750_Spy1157 | 4066696 |

Gene name = hypothetical protein; Locus tag =
Spy_1355; GeneID = 901430 Reference
sequence from = Streptococcus pyogenes M1GAS
(strain SF370, serotype M1); SEQ I.D. Nos. 45 and 46

| | | | |
|---|---|---|---|
| M1 | MGAS5005 | MGAS5005_Spy_1104 | 3571806 |
| M1 | S. pyogenes SSI-1 | SPs0830 | 1065983 |
| M28 | MGAS6180 | M28_1096 | 3574917 |
| M2 | MGAS10270 | MGAS10270_Spy1161 | 4063249 |
| M6 | MGAS10394 | M6_Spy1076 | 2940706 |
| M12 | MGAS9429 | MGAS9429_Spy1148 | 4060735 |
| M3 | MGAS315 | SpyM3_1030 | 1009345 |
| M4 | MGAS10750 | MGAS10750_Spy1203 | 4067008 |
| M5 | Manfredo | SpyM50755 | 4964203 |
| M18 | MGAS8232 | SpyM18_1367 | 993838 |
| M49 | M49 591 | SpyoM01000531 | n/a |
| M12 | MGAS2096 | MGAS2096_Spy1166 | 4065126 |

Gene name = putative acetyl transferase; Locus tag =
Spy_1356; GeneID = 901431 Reference
sequence from = Streptococcus pyogenes M1GAS
(strain SF370, serotype M1); SEQ I.D. Nos. 47 and 48

| | | | |
|---|---|---|---|
| M1 | MGAS5005 | MGAS5005_Spy_1105 | 3571807 |
| M1 | S. pyogenes SSI-1 | SPs0829 | 1065974 |
| M28 | MGAS6180 | M28_1097 | 3574918 |
| M2 | MGAS10270 | MGAS10270_Spy1162 | 4063250 |
| M6 | MGAS10394 | M6_Spy1077 | 2940707 |
| M12 | MGAS9429 | MGAS9429_Spy1149 | 4060736 |
| M3 | MGAS315 | SpyM3_1031 | 1009346 |
| M4 | MGAS10750 | MGAS10750_Spy1204 | 4067009 |
| M5 | Manfredo | SpyM50754 | 4964294 |
| M18 | MGAS8232 | SpyM18_1368 | 993773 |
| M49 | M49 591 | SpyoM01000532 | n/a |
| M12 | MGAS2096 | MGAS2096_Spy1167 | 4065127 |

Gene name = putative penicillin-binding protein 1A
(pbp1A); Locus tag = Spy_1649; GeneID =
901903; Reference sequence from = Streptococcus pyogenes
M1GAS (strain SF370, serotype M1); SEQ I.D. Nos. 49 and 50

| | | | |
|---|---|---|---|
| M1 | MGAS5005 | MGAS5005_Spy_1355 | 3571548 |
| M1 | S. pyogenes SSI-1 | SPs0472 | 1065774 |
| M28 | MGAS6180 | M28_1396 | 3574116 |
| M2 | MGAS10270 | MGAS10270_Spy1472 | 4063605 |
| M6 | MGAS10394 | M6_Spy1401 | 2940852 |
| M12 | MGAS9429 | MGAS9429_Spy1351 | 4061788 |
| M3 | MGAS315 | SpyM3_1390 | 1009705 |
| M4 | MGAS10750 | MGAS10750_Spy1464 | 4067496 |
| M5 | Manfredo | SpyM50436 | 4964314 |
| M18 | MGAS8232 | SpyM18_1661 | 994224 |
| M49 | M49 591 | SpyoM01000630 | n/a |
| M12 | MGAS2096 | MGAS2096_Spy1377 | 4065206 |

Gene name = putative ABC transporter protein; Locus
tag = Spy_1674 GeneID = 901921; Reference
sequence from = Streptococcus pyogenes M1GAS
(strain SF370, serotype M1); SEQ I.D. Nos. 51 and 52

| | | | |
|---|---|---|---|
| M1 | MGAS5005 | MGAS5005_Spy_1373 | 3571527 |
| M1 | S. pyogenes SSI-1 | SPs0406 | 1066926 |
| M28 | MGAS6180 | M28_1416 | 3574136 |
| M2 | MGAS10270 | MGAS10270_Spy1491 | 4064106 |
| M6 | MGAS10394 | M6_Spy1421 | 2942110 |
| M12 | MGAS9429 | MGAS9429_Spy1371 | 4061817 |
| M3 | MGAS315 | SpyM3_1430 | 1009775 |
| M4 | MGAS10750 | MGAS10750_Spy1483 | 4067476 |
| M5 | Manfredo | SpyM50417 | 4964572 |
| M18 | MGAS8232 | SpyM18_1685 | 993969 |
| M49 | M49 591 | SpyoM01001413 | n/a |
| M12 | MGAS2096 | MGAS2096_Spy1396 | 4066317 |

Gene name = putative transcriptional regulator; Locus
tag = Spy_1733 GeneID = 901968;
Reference sequence from = Streptococcus pyogenes
M1GAS (strain SF370, serotype M1); SEQ I.D. Nos. 53 and 54

| | | | |
|---|---|---|---|
| M1 | MGAS5005 | MGAS5005_Spy_1474 | 3571435 |
| M1 | S. pyogenes SSI-1 | SPs0361 | 1066905 |
| M28 | MGAS6180 | M28_1463 | 3574184 |
| M2 | MGAS10270 | MGAS10270_Spy1541 | 4062758 |
| M6 | MGAS10394 | M6_Spy1468 | 2942200 |
| M12 | MGAS9429 | MGAS9429_Spy1476 | 4061642 |
| M3 | MGAS315 | SpyM3_1506 | 1009821 |
| M4 | MGAS10750 | MGAS10750_Spy1533 | 4067450 |
| M5 | Manfredo | SpyM50371 | 4962971 |
| M18 | MGAS8232 | SpyM18_1741 | 994566 |
| M49 | M49 591 | SpyoM01000766 | n/a |
| M12 | MGAS2096 | MGAS2096_Spy1499 | 4064613 |

Gene name = putative ABC transporter; Locus tag =
Spy_1784; GeneID = 902015 Reference
sequence from = Streptococcus pyogenes M1GAS
(strain SF370, serotype M1); SEQ I.D. Nos. 55 and 56

| | | | |
|---|---|---|---|
| M1 | MGAS5005 | MGAS5005_Spy_1518 | 3571363 |
| M1 | S. pyogenes SSI-1 | SPs0316 | 1066465 |
| M28 | MGAS6180 | M28_1508 | 3574229 |
| M2 | MGAS10270 | MGAS10270_Spy1586 | 4063757 |
| M6 | MGAS10394 | M6_Spy1511 | 2941566 |
| M12 | MGAS9429 | MGAS9429_Spy1520 | 4060951 |
| M3 | MGAS315 | SpyM3_1550 | 1009865 |
| M4 | MGAS10750 | MGAS10750_Spy1578 | 4067379 |
| M5 | Manfredo | SpyM50327 | 4962953 |
| M18 | MGAS8232 | SpyM18_1856 | 993954 |
| M49 | M49 591 | SpyoM01000777 | n/a |
| M12 | MGAS2096 | MGAS2096_Spy1545 | 4065737 |

Gene name = streptococcal iron acquisition protein
(siaA), also known as htsA Locus tag = Spy_1795;
GeneID = 902024; Reference sequence from =
Streptococcus pyogenes M1GAS (strain SF370,
serotype M1); SEQ I.D. Nos. 57 and 58

| | | | |
|---|---|---|---|
| M1 | MGAS5005 | MGAS5005_Spy_1528 | 3571373 |
| M1 | S. pyogenes SSI-1 | SPs0307 | 1066589 |
| M28 | MGAS6180 | M28_1518 | 3574239 |
| M2 | MGAS10270 | MGAS10270_Spy1596 | 4063652 |
| M6 | MGAS10394 | M6_Spy1521 | 2941369 |
| M12 | MGAS9429 | MGAS9429_Spy1532 | 4060830 |
| M3 | MGAS315 | SpyM3_1560 | 1009875 |
| M4 | MGAS10750 | MGAS10750_Spy1587 | 4067388 |
| M5 | Manfredo | SpyM50318 | 4962943 |
| M18 | MGAS8232 | SpyM18_1867 | 993699 |
| M49 | M49 591 | SpyoM01000537 | n/a |
| M12 | MGAS2096 | MGAS2096_Spy1555 | 4065747 |

Gene name = DNA polymerase III subunit epsilon (dnaQ);
Locus tag = Spy_1864 GeneID = 902074; Reference
sequence from = Streptococcus pyogenes M1GAS
(strain SF370, serotype M1); SEQ I.D. Nos. 59 and 60

| | | | |
|---|---|---|---|
| M1 | MGAS5005 | MGAS5005_Spy_1582 | 3571310 |
| M1 | S. pyogenes SSI-1 | SPs0259 | 1065779 |
| M28 | MGAS6180 | M28_1570 | 3574272 |
| M2 | MGAS10270 | MGAS10270_Spy1649 | 4064346 |
| M6 | MGAS10394 | M6_Spy1594 | 2941328 |
| M12 | MGAS9429 | MGAS9429_Spy1587 | 4061431 |
| M3 | MGAS315 | SpyM3_1608 | 1009923 |
| M4 | MGAS10750 | MGAS10750_Spy1640 | 4066776 |
| M5 | Manfredo | SpyM50268 | 4962913 |
| M18 | MGAS8232 | SpyM18_1928 | 994533 |
| M12 | MGAS2096 | MGAS2096_Spy1607 | 4065973 |

Gene name = hypothetical protein; Locus tag =
Spy_1865; GeneID = 902075 Reference
sequence from = Streptococcus pyogenes M1GAS
(strain SF370, serotype M1); SEQ I.D. Nos. 61 and 62

| | | | |
|---|---|---|---|
| M1 | MGAS5005 | MGAS5005_Spy_1583 | 3571311 |
| M1 | S. pyogenes SSI-1 | SPs0258 | 1065783 |
| M28 | MGAS6180 | M28_1571 | 3574273 |
| M2 | MGAS10270 | MGAS10270_Spy1650 | 4064347 |
| M6 | MGAS10394 | M6_Spy1595 | 2941329 |
| M12 | MGAS9429 | MGAS9429_Spy1588 | 4061432 |
| M3 | MGAS315 | SpyM3_1609 | 1009924 |
| M4 | MGAS10750 | MGAS10750_Spy1641 | 4066777 |
| M5 | Manfredo | SpyM50267 | 4964089 |
| M18 | MGAS8232 | SpyM18_1929 | 995043 |
| M12 | MGAS2096 | MGAS2096_Spy1608 | 4065974 |

Gene name = DNA-directed RNA polymerase subunit
delta (rpoE); Locus tag = Spy_1895; GeneID =
902099; Reference sequence from = Streptococcus pyogenes
M1GAS (strain SF370, serotype M1); SEQ I.D. Nos. 63 and 64

| | | | |
|---|---|---|---|
| M1 | MGAS5005 | MGAS5005_Spy_1611 | 3571270 |
| M1 | S. pyogenes SSI-1 | SPs0233 | 1066365 |
| M28 | MGAS6180 | M28_1600 | 3574304 |
| M2 | MGAS10270 | MGAS10270_Spy1679 | 4064179 |
| M6 | MGAS10394 | M6_Spy1619 | 2941986 |
| M12 | MGAS9429 | MGAS9429_Spy1614 | 4062113 |
| M3 | MGAS315 | SpyM3_1633 | 1009948 |
| M4 | MGAS10750 | MGAS10750_Spy1666 | 4066764 |
| M5 | Manfredo | SpyM50243 | 4962899 |
| M18 | MGAS8232 | SpyM18_1960 | 994734 |
| M49 | M49 591 | SpyoM01001243 | n/a |
| M12 | MGAS2096 | MGAS2096_Spy1634 | 4066193 |

Gene name = putative tagatose 6-phosphate kinase
(lacC.2); Locus tag = Spy_1921 GeneID =
901604; Reference sequence from = Streptococcus
pyogenes M1GAS (strain SF370, serotype M1);
SEQ I.D. Nos. 65 and 66

| | | | |
|---|---|---|---|
| M1 | MGAS5005 | MGAS5005_Spy_1636 | 3571268 |
| M1 | S. pyogenes SSI-1 | SPs1655 | 1066377 |
| M28 | MGAS6180 | M28_1626 | 3574354 |
| M2 | MGAS10270 | MGAS10270_Spy1705 | 4062540 |
| M6 | MGAS10394 | M6_Spy1645 | 2941717 |
| M12 | MGAS9429 | MGAS9429_Spy1639 | 4061438 |
| M3 | MGAS315 | SpyM3_1657 | 1009972 |
| M4 | MGAS10750 | MGAS10750_Spy1733 | 4067200 |
| M5 | Manfredo | SpyM51611 | 4964245 |
| M18 | MGAS8232 | SpyM18_1989 | 994273 |
| M49 | M49 591 | SpyoM01000235 | n/a |
| M12 | MGAS2096 | MGAS2096_Spy1661 | 4064633 |

Gene name = putative ABC transporter protein; Locus
tag = Spy_2031 GeneID = 901684; Reference
sequence from = Streptococcus pyogenes M1GAS
(strain SF370, serotype M1); SEQ I.D. Nos. 67 and 68

| | | | |
|---|---|---|---|
| M1 | MGAS5005 | MGAS5005_Spy_1727 | 3571167 |
| M1 | S. pyogenes SSI-1 | SPs1732 | 1065123 |
| M28 | MGAS6180 | M28_1711 | 3574440 |
| M2 | MGAS10270 | MGAS10270_Spy1793 | 4063167 |
| M6 | MGAS10394 | M6_Spy1727 | 2940883 |
| M12 | MGAS9429 | MGAS9429_Spy1732 | 4061116 |
| M3 | MGAS315 | SpyM3_1735 | 1010050 |
| M4 | MGAS10750 | MGAS10750_Spy1818 | 4067042 |
| M5 | Manfredo | SpyM51690 | 4963859 |
| M18 | MGAS8232 | SpyM18_2089 | 994103 |
| M49 | M49 591 | SpyoM01001110 | n/a |
| M12 | MGAS2096 | MGAS2096_Spy1755 | 4064574 |

Gene name = putative ABC transporter-like protein; Locus
tag = Spy_2032 GeneID = 901685; Reference
sequence from = Streptococcus pyogenes
M1GAS (strain SF370, serotype M1); SEQ I.D. Nos. 69 and 70

| | | | |
|---|---|---|---|
| M1 | MGAS5005 | MGAS5005_Spy_1728 | 3571168 |
| M1 | S. pyogenes SSI-1 | SPs1733 | 1066253 |
| M28 | MGAS6180 | M28_1712 | 3574441 |
| M2 | MGAS10270 | MGAS10270_Spy1794 | 4063168 |
| M6 | MGAS10394 | M6_Spy1728 | 2941283 |
| M12 | MGAS9429 | MGAS9429_Spy1733 | 4061117 |
| M3 | MGAS315 | SpyM3_1736 | 1010051 |
| M4 | MGAS10750 | MGAS10750_Spy1819 | 4067043 |
| M5 | Manfredo | SpyM51691 | 4963860 |
| M18 | MGAS8232 | SpyM18_2090 | 994763 |
| M49 | M49 591 | SpyoM01001109 | n/a |
| M12 | MGAS2096 | MGAS2096_Spy1756 | 4064575 |

Gene name = hypothetical protein; Locus tag =
Spy_2060; GeneID = 901709 Reference
sequence from = Streptococcus pyogenes M1GAS
(strain SF370, serotype M1), SEQ I.D. Nos. 71 and 72

| | | | |
|---|---|---|---|
| M1 | MGAS5005 | MGAS5005_Spy_1754 | 3571117 |
| M1 | S. pyogenes SSI-1 | SPs1757 | 1066220 |
| M28 | MGAS6180 | M28_1740 | 3574488 |
| M2 | MGAS10270 | MGAS10270_Spy1823 | 4062697 |
| M6 | MGAS10394 | M6_Spy1754 | 2940874 |
| M12 | MGAS9429 | MGAS9429_Spy1764 | 4061237 |
| M3 | MGAS315 | SpyM3_1759 | 1010074 |
| M4 | MGAS10750 | MGAS10750_Spy1848 | 4066689 |
| M5 | Manfredo | SpyM51716 | 4964340 |
| M18 | MGAS8232 | SpyM18_2121 | 993712 |
| M49 | M49 591 | SpyoM01000774 | n/a |
| M12 | MGAS2096 | MGAS2096_Spy1789 | 4065148 |

Gene name = hypothetical protein; Locus tag =
Spy_2063; GeneID = 901710 Reference
sequence from = Streptococcus pyogenes M1GAS
(strain SF370, serotype M1) SEQ I.D. Nos. 73 and 74

| | | | |
|---|---|---|---|
| M1 | MGAS5005 | MGAS5005_Spy_1756 | 3571119 |
| M1 | S. pyogenes SSI-1 | SPs1758 | 1065113 |
| M28 | MGAS6180 | M28_1742 | 3574452 |
| M2 | MGAS10270 | MGAS10270_Spy1825 | 4062699 |
| M6 | MGAS10394 | M6_Spy1756 | 2940724 |
| M12 | MGAS9429 | MGAS9429_Spy1766 | 4061239 |
| M3 | MGAS315 | SpyM3_1761 | 1010076 |
| M4 | MGAS10750 | MGAS10750_Spy1850 | 4066691 |
| M5 | Manfredo | SpyM51717 | 4963879 |
| M18 | MGAS8232 | SpyM18_2124 | 994392 |
| M49 | M49 591 | SpyoM01000772 | n/a |
| M12 | MGAS2096 | MGAS2096_Spy1791 | 4065150 |

Gene name = hypothetical protein; Locus tag =
SpyM3_1257; GeneID = 1009572 Reference
sequence from = Streptococcus
pyogenes MGAS315 (strain MGAS315,
serotype M3); SEQ I.D. Nos. 75 and 76

| | | | |
|---|---|---|---|
| M1 | S. pyogenes SSI-1 | SPs0606 | 1066746 |
| M1 | S. pyogenes SSI-1 | SPs0414 | 1066929 |
| M28 | MGAS6180 | M28_1277 | 3573985 |
| M2 | MGAS10270 | MGAS10270_Spy1353 | 4063770 |
| M6 | MGAS10394 | M6_Spy0026 | 2940912 |
| M3 | MGAS315 | SpyM3_1452 | 1009767 |

Gene name = hypothetical protein; Locus tag =
SpyM3_1326; GeneID = 1009641
Reference sequence from = Streptococcus
pyogenes MGAS315 (strain MGAS315,
serotype M3); SEQ I.D. Nos. 77 and 78

| | | | |
|---|---|---|---|
| M1 | MGAS5005 | MGAS5005_Spy_1022 | 3571880 |
| M1 | S. pyogenes SSI-1 | SPs0535 | 1066759 |
| M2 | MGAS10270 | MGAS10270_Spy1825 | 4062961 |
| M4 | MGAS10750 | MGAS10750_Spy1861 | 4066847 |
| M49 | M49 591 | SpyoM01000031 | n/a |

Gene name = hypothetical protein; Locus tag =
SpyM18_1298; GeneID = 994620
Reference sequence from = Streptococcus
pyogenes MGAS8232 (strain MGAS8232,
serotype M18); SEQ I.D. Nos. 79 and 80

| | | | |
|---|---|---|---|
| M1 | S. pyogenes SSI-1 | SPs0887 | 1066078 |
| M28 | MGAS6180 | M28_1020 | 3573750 |
| M6 | MGAS10394 | M6_Spy1017 | 2941548 |
| M3 | MGAS315 | SpyM3_0966 | 1009281 |

Gene name = hypothetical protein; Locus tag =
SpyM18_1299; GeneID = 993960;
Reference sequence from = Streptococcus
pyogenes MGAS8232 (strain MGAS8232,
serotype M18); SEQ I.D. Nos. 81 and 82

| | | | |
|---|---|---|---|
| M1 | MGAS5005 | MGAS5005_Spy_1213 | 3571681 |
| M1 | S. pyogenes SSI-1 | SPs0886 | 1066065 |
| M28 | MGAS6180 | M28_1021 | 3573751 |
| M6 | MGAS10394 | M6_Spy1018 | 2942036 |
| M3 | MGAS315 | SpyM3_0967 | 1009282 |
| M5 | Manfredo | SpyM50478 | 4963038 |

1. An immunogenic composition for protecting mammals against infection by Group A Streptococcus (GAS) comprising an effective amount of a molecule selected from the group consisting of:
(a) a region of at least one Group A Streptococcus marker listed in Tables 3, 4 or 5 that defines an epitope which induces the formation of bactericidal antibodies against GAS;
(b) a polypeptide listed in Table 3, 4 or 5;
(c) a peptide derived from (a) or (b);
(d) a chemically produced, synthetic peptide derived from (a), (b) or (c); and
(e) a combination of the molecules of (a) through (d).

2. A composition as claimed in claim 1, wherein the region is immunoreactive and found in the most prevalent GAS serotypes associated with a GAS disease.

3. A composition as claimed in claim 1 comprising a synthetic peptides about 5 to 200 amino acids in length which is a portion of a polypeptide listed in Tables 3, 4 or 5.

4-5. (canceled)

6. A composition as claimed in claim 1, further comprising a pharmaceutically acceptable carriers, an excipient, a diluent, a vehicles, or another immune-stimulatory molecules.

7. A method of inhibiting or reducing the growth of Group A Streptococcus in blood or of reducing phagocytic resistance in a subject or of immunizing a human against infection by Group A Streptococcus comprising administering an effective amount of an immunogenic composition as claimed in claims 1.

8. (canceled)

9. A method for treating or preventing a GAS disease in a subject comprising administering to a subject in need thereof antibodies specific for one or more polypeptide listed in Tables 3, 4 or 5 or a composition as claimed in claim 1.

10. A method for stimulating or enhancing in a subject production of antibodies directed against one or more polypeptide listed in Tables 3, 4 or 5, comprising administering to the subject an immunogenic composition as claimed in claim 1 in a dose effective for stimulating or enhancing production of the antibodies.

11. (canceled)

12. A method as claimed in claim 14, wherein the method comprises:
(a) obtaining a sample from a subject;
(b) detecting in proteins extracted from the sample one or more GAS markers that are associated with the disease; and
(c) comparing the detected amount with an amount detected for a standard, wherein the GAS markers comprise at least one polypeptide listed in Tables 3, 4 or 5.

13. A method as claimed in claim 12 comprising:
(a) contacting a biological sample obtained from a subject with one or more antibody that specifically binds to the GAS markers or parts thereof; and
(b) detecting in the sample amounts of GAS markers that bind to the antibody relative to a predetermined standard or cut-off value, and therefrom determining the presence or absence of the GAS disease in the subject.

14. A method for determining the presence or absence of Group A Streptococcus (GAS) markers associated with a GAS disease in a subject comprising detecting one or more GAS markers or polynucleotides encoding GAS markers in a sample from the subject and relating the detected amount to the presence of a GAS disease, wherein the GAS markers comprise at least one polypeptide listed in Tables 3, 4 or 5.

15. A method as claimed in claim 14 wherein the GAS marker is a polynucleotide in Table 3, 4 or 5 or a fragment or modified form thereof.

16. A method as claimed in claim 14 wherein the polynucleotides detected are mRNA.

17. A method as claimed in claim 14 wherein the polynucleotide is detected by
(a) contacting the sample with oligonucleotides that hybridize to the polynucleotides; and
(b) detecting in the sample levels of nucleic acids that hybridize to the polynucleotides relative to a predetermined standard or cut-off value, and therefrom determining the presence or absence of a GAS disease in the subject.

18. A method as claimed in claim 16 wherein the mRNA is detected using an amplification reaction.

19. A method as claimed in claim 14 comprising reacting one or more GAS marker polypeptide in Tables 3, 4 or 5 with a test sample from a subject suspected of comprising antibodies specific for the GAS marker polypeptide under conditions that allow polypeptide/antibody complexes to form and detecting polypeptide/antibody complexes, wherein the detection of polypeptide/antibody complexes is an indication of GAS disease or infection.

20. A method as claimed in claim 19 wherein the GAS marker polypeptide comprises or consists essentially of one or more epitope of a GAS marker polypeptide of Table 4 or 5.

21. A diagnostic composition comprising
(a) an agent that binds to one or more GAS markers or hybridizes to a polynucleotide encoding such marker, wherein the GAS markers comprise at least one polypeptide listed in Tables 3, 4 or 5; or
(b) a set of markers comprising a plurality of polypeptides comprising or consisting of one or more polypeptide or polynucleotide listed in Table 3, 4 or 5.

22. A diagnostic composition as claimed in claim 21 wherein the agent is an antibody.

23-24. (canceled)


SEQUENCE LISTING (85 sequences)

SEQ ID NO: 1 - protein, 421 aa, Streptococcus pyogenes
MKLLVVGSGGREHAIAKKLLASKGVDQVFVAPGNDGMTLDGLDLVNIVVSEHSRLIAFAKENEISWAFIGPDDALAAGIVDDFNSAGLRAFGPTKAAAELEWSKDFAKEIMVKYNVPTAAYGTFSDFEKAKAYIEEQGAPIVVKADGLALGKGVVVAETVEQAVEAAQEMLLDNKFGDSGARVVIEEFLDGEEFSLFAFANGDKFYIMPTAQDHKRAFDGDKGPNTGGMGAYAPVPHLPQSVVDTAVEMIVRPVLEGMVAEGRPYLGVLYVGLILTADGPKVIEFNSRFGDPETQIILPRLTSDFAQNIDDIMMGIEPYITWQKDGVTLGVVVASEGYPFDYEKGVPLPEKTDGDIITYYAGVKFSENSELLLSNGGRVYMLVTTEDSVKAGQDKIYTQLAQQDTTGLFYRNDIGSKAIRE

SEQ ID NO: 2 - DNA, 1266 bp, Streptococcus pyogenes
ttgaaattacttgttgttggttcaggtggtcgtgaacatgcgattgctaagaagttgttagcgtctaagggtgtggatcaggtttttgtggcacctggtaatgatggtatgaccttggatggtctggacttggtgaatatagtagtttccgaacattccaggttgattgcctttgctaaggagaatgagatttcttgggcctttattggtcctgatgatgcgctagcagctggtatcgttgatgatttcaacagtgctggactcagagcttttggtccaacaaaggcagcagcggagctagagtggtcaaaagactttgccaaggaaatcatggtcaaatacaatgtcccaacagcagcctatggcacattttcagactttgaaaaagccaaagcctacatcgaagagcagggcgcacctatcgtggtcaaggctgatggcttggcgctaggtaagggtgtggtcgtggctgaaaccgttgagcaggcggtagaggcggcgcaagagatgcttctggacaataagtttggcgattcaggagcgcgtgtggttatcgaggaattcttggatggcgaggagttttccctctttgcctttgctaatggcgataagttctacatcatgccgacagctcaggatcacaagcgtgcctttgacggagacaaggggccaaacactggtggtatgggtgcctatgcgccagttcctcatctacctcagagcgtggtagacacagcggttgagatgatcgttaggcctgtccttgagggcatggttgccgaagggcgtccttatctaggtgtcctctatgtggggcttatcctgacagctgatggacctaaggttatcgagttcaactcacgtttcggtgaccctgaaactcagattatcctccctcgcctgacttctgatttcgctcagaatattgacgatatcatgatgggcattgagccttacatcacttggcagaaggacggtgtgactttgggtgttgtcgttgcctcagaaggctatccattcgattatgagaaaggtgtgccactgcctgaaaagaccgacggcgatatcatcacctactacgcaggagttaagttttcggaaaatagcgagctacttctctcaaacggaggacgtgtctatatgctcgtcaccacagaagacagcgtcaaagcagggcaggacaaaatctatacccaactggcccaacaagacacaacaggtctcttctaccgaaatgatatcggaagcaaagctataagagaataa

SEQ ID NO: 3 - protein, 281 aa, Streptococcus pyogenes
MSFKKWIGIVGIALASTVLVACSEKQDDKNTLTIGVMTKTESDQARWDKVEELLKKDNITLKYKEFTDYSQPNKAVANGEVDINAFQHYNFLNNWNKENKEHLVAIADTYISPINLFSGTSQDGKAKYKSVADLPNGTQIAVPNDATNESRALYVLQSAGLIKLNVSGDQLATIANISENKKKLDIKELDASQTARALVSADAAVVNNSYAVPAKIDYKTSLFKEKADDNSKQWINIIAGQKDWEKSEKADAIKKLIKAYQTDEVKKVVEKTSNGIDVSVW

SEQ ID NO: 4 - DNA, 846 bp, Streptococcus pyogenes
atgtcttttaaaaaatggattggaatagtcggaatcgctttagcatcaactgtattggttgcttgttctgaaaaacaagatgataaaaacactttaactattggggtgatgacaaaaacagaatctgatcaagcgagatgggataaagttgaagaattattgaaaaaagacaatatcactttgaagtacaaagaatttacagattactcgcaacctaataaggcagttgctaatggtgaagtcgatattaatgccttccaacactataatttcttaaataactggaataaggaaaataaggaacatttggtggccattgctgatacctatatcagcccaattaaccttttctctggaaccagtcaagatggaaaggctaaatacaaatcagtagctgatctgccaaatggtactcaaattgcagtaccaaatgatgcgaccaatgaaagccgtgccctctatgtgctgcaatcagctggcctcattaaattgaatgtgtctggtgatcaattagcaaccattgccaatatctcagagaacaaaaagaaattagatatcaaggaattagatgctagccaaacggcacgtgctttagtatctgctgatgcagctgttgtcaataatagttacgctgttcctgcaaaaattgactacaaaacctctcttttcaaagaaaaagcagatgacaattcaaaacagtggattaacattattgcaggtcaaaaagattgggaaaaatcagagaaggctgatgctattaaaaaactcatcaaggcctaccaaactgatgaggtgaagaaagtcgtggaaaaaacttcaaatggtatagatgtatccgtatggtaa

SEQ ID NO: 5 - protein, 196 aa, Streptococcus pyogenes
MIPVVIEQTSRGERSYDIYSRLLKDRIIMLTGPVEDNMANSVIAQLLFLDAQDNTKDIYLYVNTPGGSVSAGLAIVDTMNFIKADVQTIVMGMAASMGTVIASSGTKGKRFMLPNAEYMIHQPMGGTGGGTQQTDMAIAAEHLLKTRHRLEKILAQNAGKTIKQIHKDAERDYWMSAEETLAYGFIDEIMENNELK

SEQ ID NO: 6 - DNA, 591 bp, Streptococcus pyogenes
atgattcctgttgttattgaacaaactagccgtggagaacgttcttatgacatttattcacgtttattaaaagaccgtattatcatgttaacagggcctgtcgaagacaacatggccaactctgtcatagcgcagctcttattccttgacgcgcaagacaacactaaagatatctacctatatgtcaacacaccaggtggctctgtctcagcaggcttggctattgtcgataccatgaactttatcaaggccgatgtgcaaactattgtcatgggtatggcagcttctatggggacagtcattgcttcatcaggaacaaaaggcaaacgctttatgttaccaaatgcagagtatatgatccaccagccaatgggtggtacaggtggtggcacgcaacaaacggatatggctatcgcagcagaacatctcttaaaaacacgtcaccgtttagaaaaaatcttagcgcaaaatgctggaaaaacgattaaacaaatccataaagatgctgagcgtgattattggatgagtgctgaagaaaccttggcttacggtttcattgatgaaatcatggaaaacaacgaactaaaataa

SEQ ID NO: 7 - protein, 91 aa, Streptococcus pyogenes
MFQKQERIGLVVYLYYNRDARKLSKFGDLYYHSKRSRYLIIYINKNDLDTKLEEMRRLKCVKDIRPSAFDDIDRQFVGNLHRDETNNHQKG

SEQ ID NO: 8 - DNA, 276 bp, Streptococcus pyogenes
atgtttcaaaaacaagaacgtattggactcgtcgtttacctgtattataaccgagatgccagaaagttatcaaaatttggtgatttatattaccattccaagcgctctcgttacctcattatttatattaataaaaatgatttagacacaaaattagaagaaatgagacgtttgaaatgtgttaaagacattagaccatcagcttttgatgatattgaccgccaatttgtaggtaaccttcatcgcgacgaaacaaacaaccatcaaaagggttaa

SEQ ID NO: 9 - protein, 184 aa, Streptococcus pyogenes
MKWSGFMKTKSKRFLNLATLCLALLGTTLLMAHPVQAEVISKRDYMTRFGLGDLEDDSANYPSNLEARYKGYLEGYEKGLKGDDIPERPKIQVPEDVQPSDHGDYRDGYEEGFGEGQHKRDPLETEAEDDSQGGRQEGRQGHQEGADSSDLNVEESDGLSVIDEVVGVIYQAFSTIWTYLSGLF

SEQ ID NO: 10 - DNA, 555 bp, Streptococcus pyogenes
atgaaatggagtggttttatgaaaacaaaatcaaaacgctttttaaacctagcaaccctttgcttggccctactaggaacaactttgctaatggcacatcccgtacaggcggaggtgatatcaaaaagagactatatgactcgcttcgggttaggcgatttagaagatgattcagctaactatccttcaaatttagaagctagatataaaggatatttagagggatatgaaaaaggcttaaaaggagatgatatacccgaacggcccaagattcaggttcctgaggatgttcagccatctgaccatggcgactatagagatggttatgaggaaggatttggagaaggacaacataaacgtgatccattagaaacagaagcagaagatgattctcaaggaggacgtcaagaaggacgtcaaggacatcaagaaggagcagattctagtgatttgaacgttgaagaaagcgacggtttgtctgttattgatgaagtagttggagtaatttatcaagcatttagtactatttggacatacttaagcggtttgttctaa

SEQ ID NO: 11 - protein, 260 aa, Streptococcus pyogenes
MDINLLQALLIGLWTAFCFSGMLLGIYTNRCIILSFGVGIILGDLPTALSMGAISELAYMGFGVGAGGTVPPNPIGPGIFGTLMAITSAGKVTPEAALALSTPIAVAIQFLQTFAYTAFAGAPETAKKQLQKGNIRGFKFAANGTIWAFAFIGLGLGLLGALSMDTLLHLVDYIPPVLLNGLTVAGKMLPAIGFAMILSVMAKKELIPFVLIGYVCAAYLQIPTIGIAIIGIIFALNEFYNKPKQVDATTVQGGQQDDWI

SEQ ID NO: 12 - DNA, 783 bp, Streptococcus pyogenes
atggatatcaatttgttacaggcacttttaattggtctttggacagccttttgtttcagcggaatgttacttggcatctacaccaaccgttgtattattctgtcttttggtgtagggattatcttaggtgacttgccaactgcacttagcatgggagctatctccgaattagcttatatgggatttggagtgggtgccggtggtaccgttcctcctaatcctatcggccctggtatctttggtaccttgatggctattaccagtgctggtaaagtcaccccagaagcggcactagccttatcaacaccaatcgcagttgccatccaattccttcaaacattcgcctatacagcttttgctggcgctcccgaaaccgctaaaaaacaattgcaaaaaggcaatattagaggcttcaaattcgctgccaacggcactatctgggctttcgcttttatcggattaggccttggtttattaggtgccttgtcaatggatactctccttcacttggttgattatatcccacctgtcttgctcaatggattgactgtcgctggtaaaatgttacctgctatcggatttgccatgatcttatctgttatggccaagaaagaattgattccttttgtactaattggttatgtttgtgcagcctacctccaaattccaaccattggtatcgccattattggtatcattttcgccttgaatgaattttacaacaaacctaaacaagtcgatgcaacaactgtccaaggaggccaacaagatgactggatctaa

SEQ ID NO: 13 - protein, 162 aa, Streptococcus pyogenes
MTQPNIIMTRVDERLIHGQGQLWVKFLNCNTVIVANDAVSEDKIQQSLMKTVIPSSIAIRFFSIQKVIDIIHKASPAQSIFIVVKDLQDAKLLVEGGVPITEINIGNIHKTDDKVAITQFISLGETDKSAIRCLAHDHHVVFNTKTTPAGNSASDVDILDYI

SEQ ID NO: 14 - DNA, 489 bp, Streptococcus pyogenes
atgacacaaccaaacattatcatgacgcgagttgatgagcgtttgattcacggacaggggcaattatgggtcaaattcctcaactgcaacaccgttatcgtcgctaacgatgcggtttctgaagataagattcaacaatccctcatgaaaacagttattccatccagtattgccattcgttttttctccattcaaaaagtgattgacatcattcataaagcaagtcctgctcaaagtatcttcatcgtggtcaaagatttgcaagatgccaaactcctagttgaagggggtgttcccatcacagaaatcaatatcggtaatattcataaaactgatgataaggttgcgattacccagtttatttcgctgggagaaacagacaagtctgctattcgttgcttagcccatgaccaccacgtggtttttaacaccaaaacaactccagctggcaatagcgcctctgacgttgacatcttagattatatttaa

SEQ ID NO: 15 - protein, 337 aa, Streptococcus pyogenes
MEEEIALLENHMTEPDFWNDNIAAQKTSQELNELKGKYDTFHNMQELSDETELLLEMLDEDDSLKEELEENLMQLDKIMGAYEMTLLLSEPYDHNNAILEIHPGSGGTEAQDWGDLLLRMYTRFGNANGFKIEVLDYQAGDEAGIKSVTLSFEGPNAYGLLKSEMGVHRLVRISPFDSAKRRHTSFASVEVMPELDDTIEVEVRDDDIKMDTFRSGGAGGQNVNKVSTGVRLTHIPTGIVVSSTVDRTQYGNRDRAMKMLQAKLYQLEQEKKAQEVDALKGDKKEITWGSQIRSYVFTPYTMVKDHRTNFELAQVDKVMDGEINGFIDAYLKWRIED

SEQ ID NO: 16 - DNA, 1014 bp, Streptococcus pyogenes
ttggaagaggagattgctcttcttgaaaatcacatgactgagccagacttttggaatgacaatattgcagcgcaaaagacatctcaagaactaaatgagttaaaaggtaagtatgatactttccataatatgcaagaattatctgacgaaacagaactcttacttgagatgcttgacgaagatgattctttgaaagaagaattagaagaaaatctaatgcagctagataagataatgggtgcttatgaaatgacgcttctcttatctgaaccctatgaccacaataatgctatcttagaaatccatcctggttcaggaggaactgaagcgcaagattggggagatttactcttacgaatgtatacccgttttggcaatgctaatggtttcaaaatagaagtattggattatcaagcaggagatgaagctggtattaaatcggttaccctctcttttgaaggccccaatgcttatggtttgttgaaatcagaaatgggagttcatcgcctcgttcgtatatcaccttttgattcggctaaacgtcgtcatacctcatttgcttcagttgaagtgatgccagagttagatgatacgattgaagtagaagtacgtgatgatgacattaaaatggatactttccgttccggaggagctggtggtcagaatgtcaataaagtatcaacaggtgttcgtttgacgcatattccaactggaattgtggtatcgtctaccgttgaccgtactcaatatggaaaccgcgatcgcgctatgaagatgttgcaggctaagctatatcagttagaacaagaaaagaaagcacaagaagtagatgctctcaagggtgataaaaaagaaatcacttggggaagtcagattcgttcttatgttttcaccccttatacgatggtgaaggatcatcgtactaactttgagcttgctcaggtagataaggttatggatggagagatcaatggctttattgatgcctaccttaagtggcgcattgaagactaa

SEQ ID NO: 17 - protein, 230 aa, Streptococcus pyogenes
MALIEMSGVTKKYRRSTTALRDVNVSVNQGEFVYLVGPSGAGKSTFIKLLYREEQLTTGKLYVGEFNLTKLKARDVPILRRHIGVVFQDYKLLPRKTVFENVAYAMEVIGEKRRHIKKRVPEVLDLVGLKHKMRSFPSQLSGGEQQRVAIARAIVNNPKLLIADEPTGNLDPEISWEIMQLLERINVQGTTILMATHNSHIVNTFRHRVVAIEDGRIVRDEEKGDYGYDD

SEQ ID NO: 18 - DNA, 693 bp, Streptococcus pyogenes
atggcactaatagaaatgtctggtgttactaaaaaataccgccgctcaacgaccgccctcagagacgtcaatgtttctgttaaccaaggtgagtttgtttatctcgtaggcccatcaggtgctggtaaatcaacttttatcaaactcctttaccgtgaagaacaactgacaactggtaagctatatgtgggtgaattcaatttaactaagctaaaagcacgtgatgttccgattcttcgtcgtcacatcggagtagtttttcaagattacaaactgctaccaagaaaaacagtctttgaaaatgtagcttacgccatggaagttattggtgaaaagcgccgccacattaaaaaacgcgtgccagaggtactcgatttagttggcttgaaacataagatgcgttcttttccaagtcagctatcaggaggggagcagcaacgtgtggccattgcgcgtgcgattgttaacaaccctaaattattgattgcagatgaaccgacagggaatttagatcctgagatttcatgggaaattatgcagttacttgaacgtattaatgttcagggaacaactatcttaatggcgacacacaacagtcatattgtgaatacattccgtcatcgtgttgttgctattgaagatggtcgcatagtacgtgatgaagaaaaaggagattatggttacgatgattag

SEQ ID NO: 19 - protein, 2045 aa, Streptococcus pyogenes
MRKVKKVFVSSCMLLTVGLGVAVPTGFSQSNGVMVVKAAEVPATDLSRQASDSERVDESSLLQKENLSVDSFKLENLNGWEAENDTAGNLGKFKDPDSSGYQNILTSSGKNISVAVAPKGSGKMNIKVTKRSNFQGGYYVGGLRTQTPVLKLNDVYRYSFTTKKLSGNSSEFKTRVKPVESNNKLGKELVIRVDNKNVSTKHDWLPDISDGTHTVDFTGLDKKLSVAFRFSPRQTSNVVYEFSNINIKNISPASVPAIPSKVLEGTSVLSGTAISSGDTLEKRKSFDGDILRVYKDSKIIARTVIKGNKWDVKLSKPLIAGEKLDFEILHPRSQNVSKKISKQVEAKPFDPASYKEKVIAKLKPVYEATSEKITNDAWLDENAKDLQKQKLEEQYISGKVAISEAGTKQEAIDAAYNKYSSQTDPDSLPSQYKQGNKENEQEKGRQDLIQTRDLTLKAIQEDKWLTEQEKTIQKEEALKAFETGIESVNQTVSLEQLKQRLIVYKASEKDSEKKEYPESIPNQHIPGKEKEVKAAKQEELKKLHDTTLEKINQDKWLTPDQQAEQLKQAEVTFKKGQEAIKSAQTLTQLETDLADYVSENEGKGNSIPDKYKSGNKDDLVNKAEVKLKEAHEATKQAIEKDPWLSPEQKKAQKEKAKARLDEGLKALKAADSLEILKVTEEAFVDKEKNPDSIPNQHKAGTADQARKQALDSLDKEVQKELESIDNDNTLTTDEKAAAKKKVNDAYDVAKQTAMEANSYEDLTTIKDEFLSNLPHKQGTPLKDQQSDAIAELEKKQQEIEKAIEGDKTLPRDEKEKQIADSKERLKSDTQKVKDAKNADAIKKAFEEGKVNIPQAHIPGDLNKDKEKLLAELKQKADDTEKAIDVDKTLTEDEKKEQKVKTKAELEKAKTDVKNTQTREELDKKVPELKKAIEDTHVKGNLEGVKNKAIEDLKKAHTETVAKINGDDTLDKATKEAQVKEADKALAAGKDAITKADDADKVSTAVTEHTPKIKAAHKTGDLKKAQVDANTALDKAAEKERGEINKDATLTTEDKAKQLKEVETALTKAKDNVKAAKTADAINDARDKGVATIDAVHKAGQDLGARKSGQVAKLEEAAKATKDKISADPTLTSKEKEEQSKAVDAELKKAIEAVNAADTADKVDDALGEGVTDIKNQHKSGDSIDARREAHGKELDRVAQETKGAIEKDPTLTTEEKAKQVKDVDAAKERGMAKLNEAKDADALDKAYGEGVTDIKNQHKSGDPVDARRGLHNKSIDEVAQATKDAITADTTLTEAEKETQRGNVDKEATKAKEELAKAKDADALDKAYGDGVTSIKNQHKSGKGLDVRKDEHKKALEAVAKRVTAEIEADPTLTPEVREQQKAEVQKELELATDKIAEAKDADEADKAYGDGVTAIENAHVIGKGIEARKDLAKKDLAEAAAKTKALIIEDKTLTDDQRKEQLLGVDTEYAKGIENIDAAKDAAGVDKAYSDGVRDILAQYKEGQNLNDRRNAAKEFLLKEADKVTKLINDDPTLTHDQKVDQINKVEQAKLDAIKSVDDAQTADAINDALGKGIENINNQYQHGDGVDVRKATAKGDLEKEAAKVKALIAKDPTLTQADKDKQTAAVDAAKNTAIAAVDKATTTEGINQELGKGITAINKAYRPGEGVKARKEAAKADLEKEAAKVKALITNDPTLTKADKAKQTEAVAKALKAAIAAVDKATTAEGINQELGKGITAINKAYRPGEGVKARKEAAKADLEREAAKVREAIANDPTLTKADKAKQTEAVAKALKAAIAAVDKATTAEGINQELGKGITAINKAYRPGEGVEAHKEAAKANLEKVAKETKALISGDRYLSETEKAVQKQAVEQALAKALGQVEAAKTVEAVKLAENLGTVAIRSAYVAGLAKDTDQATAALNEAKQAAIEALKQAAAETLAKITTDAKLTEAQKAEQSENVSLALKTAIATVRSAQSIASVKEAKDKGITAIRAAYVPNKAVAKSSSANHLPKSGDANSIVLVGLGVMSLLLGMVLYSKKKESKD

SEQ ID NO: 20 - DNA, 6138 bp, Streptococcus pyogenes
atgcgtaaggtcaaaaaagtctttgttagttcatgtatgcttttaacagtgggcctcggagttgccgtacctactggattcagccaatctaatggcgtgatggttgtaaaggctgcggaagtgccggcgacagatttatcacgtcaggcgtctgattcggagagggtagatgaatcgtctttattgcagaaagaaaacttatcagtagattcatttaaattagaaaatttaaatggatgggaagctgaaaatgatacagcaggtaatttggggaaatttaaagatccagatagttcgggctatcaaaatattttgacatcatctggaaagaatatcagtgtagctgttgctcccaaaggttcaggtaaaatgaacattaaagtaactaaaagatcaaattttcagggtggatattatgtaggtggtcttagaactcaaactccggtattgaagttaaatgatgtttatcgatattcttttacaactaaaaaattatcaggaaattcttcagagttcaaaacgagagttaagcccgttgaatctaataataaactagggaaagagcttgttattagggtggataataaaaatgtatctactaagcatgattggcttccagacatctctgatggaactcatactgtggacttcactggtcttgataaaaaattatctgttgctttcagattttctccaagacaaacttcgaatgttgtttacgaattttctaacataaatataaaaaacattagtcctgcatcagtgccggctattccttcgaaagttttagagggaaccagcgtcttgtcgggtactgcaatatcttctggagatacattagaaaaaagaaaatcgtttgatggcgatatcctaagagtttataaagatagcaaaatcattgctagaacagtaataaaaggcaataagtgggatgttaaactttcaaagcctcttattgcaggtgaaaaattagattttgagattttgcatccgagatctcaaaacgttagtaaaaaaatttcaaaacaagtcgaagctaaaccatttgatccagcttcctataaagaaaaagttatagccaaattaaagccggtttatgaagctactagtgaaaaaatcacaaatgatgcttggttggatgaaaatgcgaaggatttgcaaaaacaaaaattagaagaacaatatatttctggaaaagtagcgatatcagaggctggaactaaacaagaagctatagatgcagcatataataaatattcaagtcaaacagatccagactctcttcctagtcagtataaacaaggtaataaagaaaatgaacaagaaaaagggcgtcaagatttaatccagactcgtgatctgacgttgaaagccattcaagaagacaaatggctaacagagcaggagaaaacaattcaaaaagaagaagcattaaaagcttttgaaactggtatagaaagtgttaatcaaacagtatcattagaacagttgaagcaacggttaatagtgtataaagcttctgaaaaagattcagagaaaaaagaatatcctgagtcaattcctaatcagcatattccagggaaagaaaaagaagttaaagctgctaaacaagaagaacttaaaaaattacatgacacaactcttgaaaaaatcaatcaagataaatggttgacgccagaccaacaagctgaacagttaaaacaagcggaagttacttttaaaaaaggccaagaagcaattaaaagtgctcagactttaactcagcttgagacagacttagctgattatgtttctgagaatgaaggtaagggaaattctattcccgataaatacaaatctggcaataaagatgatttggtaaataaggctgaagttaaacttaaggaagctcacgaagctactaaacaagcaattgaaaaagatccatggttgagtccggaacagaaaaaagctcaaaaagaaaaagccaaagcaagactagatgagggcttgaaagctcttaaagctgcagatagtttagagattcttaaagtgacagaagaagctttcgttgataaagaaaaaaatccagattcaattccaaatcaacataaagctggaactgctgatcaagctagaaaacaagctttagatagtttagataaggaggttcaaaaagagttagagtcaattgataacgataatactctaacaactgatgagaaagcagctgctaagaaaaaagtcaatgacgcttatgatgtagctaagcaaacagctatggaagccaattcttatgaagatttgactactattaaagatgagttcttatctaatttacctcataaacaaggaacgccgcttaaagatcaacaatctgatgctattgcagaattagagaagaagcagcaagaaattgaaaaagctattgagggtgataaaacattaccaagagacgaaaaagagaaacaaattgctgactctaaggaacgcttaaaatctgacacgcaaaaagttaaagatgctaaaaatgctgatgctattaaaaaagcatttgaagaagggaaagtgaatattcctcaagcacatatcccaggtgatttgaacaaggataaagaaaaacttcttgcagaattgaagcaaaaagcagatgatactgaaaaagctattgatgttgataaaactctgacagaagatgagaaaaaagagcaaaaagtcaaaacaaaagctgaacttgaaaaagctaaaactgatgttaaaaatactcagacacgtgaagaactagataaaaaagttccagaacttaagaaagctattgaagacactcacgttaaaggtaatcttgaaggtgttaagaataaggctattgaagatcttaaaaaagctcatactgaaacagttgctaaaataaatggtgatgatacccttgacaaagctactaaagaagctcaagtgaaagaagctgacaaagctttggcagcaggtaaagatgcgatcactaaagcagatgatgctgataaagtaagtacagctgttacagagcacacaccaaaaattaaagcagcacataaaactggtgaccttaaaaaagctcaagtagatgctaacacagctcttgacaaagcagctgaaaaagaacgtggagaaatcaataaagatgctacactaacgacagaagataaagcaaaacaactgaaagaagttgagacagctcttactaaagctaaagataacgtgaaagctgctaagacagcagacgctatcaatgacgcacgtgataaaggcgtagcaacaattgatgccgtccataaagcaggtcaagacttaggtgctcgtaagtcaggtcaagtcgctaaacttgaagaagcagctaaagcaacgaaagacaagatttcagctgatccaactttgacaagcaaagaaaaagaagagcaatctaaagctgttgacgctgaacttaagaaagcgatagaagctgttaacgcagctgacacagctgacaaggttgacgatgctcttggtgaaggtgttacagacatcaagaaccaacacaagtcaggtgactctatcgacgctcgtcgtgaggctcatggtaaggaacttgatagagtcgctcaagaaactaaaggtgcgattgaaaaagaccctactttgacgactgaagaaaaagctaaacaagttaaagacgtagatgccgctaaagaaagaggcatggctaagcttaatgaagctaaagatgctgatgctttagacaaagcttacggtgaaggtgttacagacatcaagaaccaacacaagtcaggtgaccctgtcgacgctcgtcgtggattacacaacaagtcaatcgacgaagtggcgcaagcaactaaggacgctatcacagcagatacgactttgactgaagctgaaaaagaaacacaacgtggcaatgttgataaagaagcaactaaagctaaagaagaacttgctaaggctaaagatgctgatgctttagacaaagcgtacggtgacggtgtaaccagcatcaagaaccaacacaagtcaggtaaaggtcttgacgttcgtaaagatgagcacaagaaagctcttgaagctgttgctaaacgtgtcactgctgaaattgaggctgatccaaccttaacaccagaagtgagagaacaacaaaaagcagaggttcaaaaagagcttgaacttgcgactgataagattgctgaagctaaagatgcagatgaagcagacaaagcttacggtgacggtgtcacagcgatcgaaaatgcccacgttattggtaaaggtatcgaagctcgtaaagaccttgctaagaaagaccttgctgaagctgctgctaagacaaaagctctcattattgaagacaaaacgcttactgatgatcaacgtaaagaacagttattaggtgttgatacagagtatgctaaaggtatcgagaatattgatgcagctaaggatgctgcaggtgttgataaagcatatagtgacggtgttcgtgacatcctggcacagtacaaagaaggtcaaaaccttaatgatcgtcgtaatgctgccaaagaatttcttcttaaagaagcagacaaagtgacgaaactaatcaatgatgatccaaccttgactcatgaccaaaaagttgatcaaatcaacaaagttgaacaagctaagttagacgcaatcaagtctgttgatgatgctcaaacagctgatgctatcaatgatgctcttggtaagggtattgaaaacatcaacaaccaataccaacatggcgatggcgttgatgttcgtaaagcgactgccaaaggcgatcttgaaaaagaagctgctaaagtgaaagctcttattgctaaggatccgaccttaactcaagctgataaagacaaacaaacagcagcggttgacgcagctaagaatacagcaattgcagcggttgataaagcgacaacaactgaaggcattaaccaagaacttggtaaaggcatcacagctatcaataaagcttaccgtccaggtgaaggtgttaaagcacgtaaagaagccgctaaagctgatcttgaaaaagaagctgctaaagtgaaagctcttattactaacgacccaaccttaacaaaagctgataaagctaaacaaacagaagctgttgctaaagcccttaaagctgctatcgcagcggttgataaagcgacaacagctgaaggcattaaccaagaacttggtaaaggcatcacagctatcaataaagcttaccgtccaggtgaaggtgttaaagcacgtaaagaagccgctaaggctgatcttgaaagagaagctgctaaggttcgtgaagctatcgctaacgacccaaccttaacaaaagctgataaagctaaacaaacagaagctgttgctaaagctcttaaagctgctatcgcagcggttgataaagcgacaacagctgaaggcattaaccaagaacttggtaaaggcatcacagctatcaacaaagcttaccgtccaggtgaaggtgttgaagcacataaagaagctgctaaagctaatcttgaaaaagtagctaaagaaactaaagctcttatttcaggagaccgttacttgagcgaaactgaaaaagcagtccaaaaacaagctgttgagcaagctcttgcgaaagcacttggtcaagttgaggctgctaagacagttgaagctgttaagttggcagaaaaccttggtactgtagctatccgttcagcatatgttgctggtttagctaaagatactgatcaagcaacagctgctcttaacgaagcgaaacaagctgctattgaagctcttaaacaagctgcggcagaaacacttgctaagattacaactgatgctaaattgactgaagctcaaaaagctgaacaatcagaaaatgtatcattagcgcttaagacggctattgcgactgttcgttcagcacaatctattgcgtctgtgaaagaagcaaaagataaaggtattactgctatccgtgcagcctatgtgcctaataaggcagtcgcaaaatcatcgtcagcgaaccatcttccaaaatcaggtgatgcaaactcaattgttcttgttggcttaggagttatgtctcttcttttaggtatggtgctttatagcaagaaaaaagaaagtaaagactaa

SEQ ID NO: 21 - protein, 1210 aa, Streptococcus pyogenes
MISFAPFLSPEAIKHLQENERCRDQSQKRTAQQIEAIYTSGQNILVSASAGSGKTFVMVERILDKILRGVSIDRLFISTFTVKAATELRERIENKLYSQIAQTTDFQMKVYLTEQLQSLCQADIGTMDAFAQKVVSRYGYSIGISSQFRIMQDKAEQDVLKQEVFSKLFNEFMNQKEAPVFRALVKNFSGNCKDTSAFRELVYTCYSFSQSTENPKIWLQENFLSAAKTYQRLEDIPDHDIELLLLAMQDTANQLRDVTDMEDYGQLTKAGSRSAKYTKHLTIIEKLSDWVRDFKCLYGKAGLDRLIRDVTGLIPSGNDVTVSKVKYPVFKTLHQKLKQFRHLETILMYQKDCFSLLEQLQDFVLAFSEAYLAVKIQESAFEFSDIAHFAIKILEENTDIRQSYQQHYHEVMVDEYQDNNHMQERLLTLLSNGHNRFMVGDIKQSIYRFRQADPQIFNQKFRDYQKKPEQGKVILLKENFRSQSEVLNVSNAVFSHLMDESVGDVLYDEQHQLIAGSHAQTVPYLDRRAQLLLYNSDKDDGNAPSDSEGISFSEVTIVAKEIIKLHNDKGVPFEDITLLVSSRTRNDIISHTFNQYGIPIATDGGQQNYLKSVEVMVMLDTLRTINNPRNDYALVALLRSPMFAFDEDDLARIALQKDNELDKDCLYDKIQRAVIGRGAHPELIHDTLLGKLNVFLKTLKSWRRYAKLGSLYDLIWKIFNDRFYFDFVASQAKAEQAQANLYALALRANQFEKSGYKGLYRFIKMIDKVLETQNDLADVEVATPKQAVNLMTIHKSKGLQFPYVFILNCDKRFSMTDIHKSFILNRQHGIGIKYLADIKGLLGETTLNSVKVSMETLPYQLNKQELRLATLSEEMRLLYVAMTRAEKKVYFIGKASKSKSQEITDPKKLGKLLPLALREQLLTFQDWLLAIADIFSTEDLYFDVRFIEDSDLTQESVGRLQTPQLLNPDDLKDNRQSETIARALDMLEAVSQLNANYEAAIHLPTVRTPSQLKATYEPLLEPIGVDIIEKSSRSLSDFTLPHFSKKAKVEASHIGSALHQLMQVLPLSKPINQQTLLDALRGIDSNEEVKTALDLKKIESFFCDTSLGQFFQTYQKHLYREAPFAILKLDPISQEEYVLRGIIDAYFLFDDHIVLVDYKTDKYKQPIELKKRYQQQLELYAEALTQTYKLPVTKRYLVLMGGGKPEIVEV

SEQ ID NO: 22 - DNA, 3633 bp, Streptococcus pyogenes
gtgatttcttttgccccatttttaagccccgaagctattaaacatttgcaagaaaacgaaaggtgcagagatcagtctcaaaaacgcacagctcaacaaattgaagcaatttatactagtggccaaaatatacttgtatcagcttctgctggttcaggaaaaacctttgtaatggtcgaacgcatacttgataaaattttgagaggtgtttcaattgatcggctttttatctcaacctttactgttaaagcagctacagaactgcgtgagcggattgaaaacaaattatactcacaaattgctcaaactacagattttcaaatgaaagtttatttaacagaacaattgcaatctctttgtcaagctgatattggtactatggatgcttttgcacagaaagtagtaagtcgctatggttatagcattggcatttcatcccaatttcgtatcatgcaggataaagcagaacaagatgttttaaagcaagaggtgtttagcaaactctttaatgagtttatgaatcaaaaagaggcaccggtgtttagggctcttgtgaaaaatttttctggtaactgtaaagacacttcagcttttagagagttagtttatacttgttattcttttagccaatcgacagaaaacccaaaaatatggttgcaagaaaattttctaagcgctgctaaaacttaccaaagacttgaagatatcccggatcatgatattgaactcttacttttggcaatgcaagacactgcaaatcagctaagagatgtgactgatatggaagattatgggcagctgactaaggcaggtagccgatctgctaaatacactaaacacttaacgatcatagaaaagttgtctgattgggtgcgtgattttaaatgtttgtatggaaaagccggattggatcggttgatcagagatgtgacaggccttataccatctgggaatgatgttacagtctcgaaggtaaaataccctgtttttaagaccttgcatcaaaaattaaaacaatttaggcatttagaaacaattttaatgtatcagaaagactgtttttccttattggaacagttacaagattttgtgcttgcgttttcagaagcttatttagctgtcaaaatacaagaaagtgcttttgaattttcagatattgcacactttgcaatcaaaattttagaggaaaatacggatattcgccaatcctatcagcaacactatcatgaggtgatggttgatgaatatcaagataacaatcatatgcaagagcgactcctgaccttactatcgaacggtcataatcgctttatggtaggagatatcaaacaatcgatctatcgatttcggcaagccgatcctcagatttttaatcaaaagtttagagactatcaaaaaaaacctgagcaggggaaagtgattttactcaaagaaaactttcgtagccaatcagaggtgttaaatgtcagcaatgctgtttttagtcacttaatggacgaatcagtaggagacgtcttatacgatgagcaacatcagttaatagcaggtagtcatgctcaaacagtcccctatctagaccgtcgtgctcagttattgctatataatagcgataaagatgatggcaacgccccttcagatagtgagggtatttcatttagtgaggttacaattgttgccaaagaaattattaagcttcacaatgataagggtgtcccttttgaagacattacgttactcgtttcttcaagaacaagaaatgatatcatttctcatacattcaatcaatatggtattcctatagcaacagatggtgggcagcaaaactatcttaaatctgttgaagtgatggttatgttagatacattacgcaccattaataacccaagaaatgattatgcccttgtggctttactgcgctcaccgatgtttgcctttgatgaggatgatttagcaagaatagcacttcaaaaagacaatgagctagataaagattgcctatatgacaagatacaaagggctgtgattggaagaggtgctcatcctgaattgattcacgataccttgcttggcaagttaaatgtttttttaaagacgttgaaaagctggcgtcgatacgctaagctagggtcgttgtatgacttgatttggaaaatttttaatgatcgtttttattttgattttgtagctagtcaagcaaaagcagaacaagcacaagctaatctatacgcattagctctacgtgctaatcagtttgaaaaatcgggctataaagggctataccgttttattaaaatgattgataaggtacttgagacgcaaaatgacttagctgatgtggaagtggctactcctaaacaagctgttaatttaatgaccattcacaagtctaaaggtttacaatttccgtatgtatttatccttaattgtgacaagcgcttctcaatgacagatattcataaatcatttattctgaatcggcagcacggtatcggtatcaagtaccttgcagatatcaaaggtttacttggtgaaacaacactcaattctgttaaagtaagcatggaaaccttaccttatcaattgaacaaacaagagttgcgcttagcaactttatcagaagaaatgcgcttactgtatgttgctatgacacgagctgaaaaaaaagtttattttattggtaaagctagtaagagcaaaagtcaagaaatcacagatcctaaaaagttaggcaaacttttgccgctggctttacgagaacagttattgacattccaagattggctattagcaatagcagatatattttcaactgaagatctttattttgatgttcgctttattgaagatagtgatttgacacaagagtcagtcggacgacttcaaacaccacagttattaaatccagatgatcttaaagataatcgtcaatcagaaacaattgcacgggctttagatatgttagaagcagtgtctcaattgaatgccaattatgaagcagctattcatttgccaacagttcgaacgcctagccaacttaaggcaacttacgagcctttattagaacccattggtgtagatattatagagaaatcttctcgatcgctatctgattttactttgccacatttttcaaaaaaagcaaaagttgaagcaagtcatattggatcagctcttcatcagttgatgcaggtgctccctttgtcaaaaccgataaatcaacaaacgcttttagacgctttaagaggaattgatagtaacgaagaggtaaaaacagctcttgatctcaaaaaaatagagtcgttcttttgtgatacaagcctaggccaattttttcagacttaccaaaaacacttgtatcgagaagcgccatttgctattttaaaacttgaccctatcagtcaagaagagtatgtcctacgtggtattatagatgcctactttttgtttgatgatcatattgtattagtggactataaaacagataaatacaagcagcccattgagttaaaaaagcgttaccaacaacagttggagttatatgcagaagctctcactcaaacgtataaacttcctgtgactaagcgctatcttgttttaatgggaggtggaaagccagaaattgtcgaagtttaa

SEQ ID NO: 23 - protein, 397 aa, Streptococcus pyogenes
MPSEFQKALPILTKIKEAGYEAYFVGGSVRDVLLERPIHDVDIATSSYPEETKAIFNRTVDVGIEHGTVLVLENGGEYEITTFRTEDIYVDYRRPSQVSFVRSLEEDLKRRDFTVNALALDENGQVIDKFRGLIDLKQKRLRAVGKAEERFEEDALRIMRGFRFAASLDFDIEAITFEAMRSHSPLLEKISVERSFTEFDKLLMAPHWRKGISAMIACQAYDYLPGLKQQEAGLNHLIVSLKDNFTFSDYHQAWAYVMISLAIEDPKSFLKAWKTSNDFQRYVTKLIALYRIRQERSFEKLDIYQYGKKMASLVEDLRKAQSLSVDMDRINTLDQALVIHDKHDIVLNGSHLIKDFGMKSGPQLGLMLEKVELAIVEGRLDNDFTTIEAFVREELAT

SEQ ID NO: 24 - DNA, 1194 bp, Streptococcus pyogenes
atgccttctgagtttcagaaggctttacccatattgactaaaataaaagaagctggttatgaagcctattttgtgggaggaagcgtcagagatgttttactcgagcgtcccattcatgatgtagatattgcaactagttcctatcctgaagagactaaagcgatttttaacagaactgtcgatgttggcattgaacatggtacagtgctcgtgcttgaaaatggcggtgagtatgaaattacaacctttaggacagaagatatctatgttgattatcgccgtcctagtcaggtttcttttgtgcgctctttagaggaagacttaaaacgaagagactttaccgtcaatgccttggccttggatgaaaatggtcaagttattgataaatttagaggactcattgatttaaagcaaaaacggttacgagcagtgggtaaagcagaggaacgttttgaagaagatgcactacgcattatgagaggctttcgatttgcggcgagtctggattttgacattgaagctataacttttgaagctatgaggagccattcacctctattggaaaagatttctgtggaacgttcttttactgaatttgataaattattgatggcaccgcactggcgaaaagggatttctgccatgattgcttgtcaggcttacgactatttaccaggcttgaagcagcaggaagcaggcttgaatcatctgatagtttctttgaaagataactttacctttagtgattatcaccaagcttgggcttatgtgatgattagtttagccattgaggatcctaaaagcttcctaaaagcatggaagacttcaaatgattttcagcgttatgtcacaaagttgattgctctttatagaattcgtcaggaaagatcttttgaaaaattggatatttaccagtacggcaagaaaatggcgagtctagtggaagacctccgaaaagctcaaagcttatccgttgacatggatcgtattaatactttagatcaggcactggttattcatgacaaacacgatatcgtccttaatggttctcacctgattaaggattttgggatgaaatcaggtcctcagctagggcttatgcttgagaaagttgaattagcgattgtagaggggcgcctagataatgatttcactacaattgaagcatttgttagagaggaattagcaacatga

SEQ ID NO: 25 - protein, 425 aa, Streptococcus pyogenes
MTYEETLEWIHDHLVFGIKPGLKRMLWVLGQLGNPQKNVKGVHIVGTNGKGSTVNHLQHIFTTAGYEVGTFTSPYIMDFKERISINGRMISEKDLVIAANRIRPLTERLVQETDFGEVTEFEVITLIMFLYFGDMHPVDIAIIEAGLGGLYDSTNVFQAMVVVCPSIGLDHQAILGETYANIAAQKAGVLEGGETLVFAVENPSAREVFLTKAEQVGASIWEWQEQFQMAENASGYRFTSPLGVISDIHIAMPGHHQVSNAALAIMTCLTLQDRYPRLTPDHIREGLANSLWLGRTELLAPNLMIDGAHNNESVAALVAVLKNNYNDKKLHILFGAIDTKPIADMLVALEQIGDLQVTSFHYPNAYPLEKYPERFGRVADFKDFLALRKHAKADDFFVITGSLYFISEIRQYWEKHIEKSVLLTH

SEQ ID NO: 26 - DNA, 1278 bp, Streptococcus pyogenes
atgacttatgaagagactctagaatggattcatgatcacttagttttcggcatcaagccaggcttaaaacgtatgctttgggttcttggacagttaggaaatccccaaaaaaatgtcaagggagtgcatatcgttggaactaacggaaaaggttccacggtcaatcatttacaacacatctttacgacagctggttatgaagtggggacctttacctctccttatattatggatttcaaagaacgtattagcataaatggtcgaatgatttcagaaaaagatttggttattgctgctaatcgtattcgtcctttgacagagcgtctagttcaagagactgattttggagaggtaactgaatttgaagtgattactttgatcatgttcctctattttggtgacatgcatcctgtagatattgctattattgaagcaggccttggcggtctttatgattcgaccaatgtttttcaagcaatggttgtggtctgtccctcaattggtttagatcaccaagctattttgggcgagacttacgctaatattgccgcccaaaaagctggtgttttagaagggggcgagacccttgtttttgccgttgaaaacccgtcagctcgagaggtttttttaacaaaagctgagcaggttggtgcttcaatctgggaatggcaagagcagtttcaaatggctgagaacgcatctggctatcgttttactagtcctttaggagttatttcagatattcacatcgctatgccaggacaccaccaagtgtctaatgcagctttagctattatgacttgcttaaccttgcaagaccgctatcctaggctaactccagatcatataagggaaggtctagcaaatagtttgtggctaggccgtaccgaattattagcaccaaatctaatgattgatggggctcataacaatgaaagcgttgctgctttggtagccgtcttaaaaaataactacaatgataaaaagcttcatattttgtttggtgcgatcgacaccaagcctattgcagatatgttggttgctcttgagcaaataggtgacttgcaggttactagctttcattaccctaacgcctatccattggaaaaatacccagaacgttttggtagggttgctgatttcaaagatttcttggccttgcgtaagcatgctaaagcagatgactttttcgtgattacagggtcactatattttatttcagaaattagacagtattgggaaaaacatattgaaaaaagcgttcttttaacccattaa

SEQ ID NO: 27 - protein, 357 aa, Streptococcus pyogenes
MRKLYSFLAGVLGVIVILTSLSFILQKKSGSGSQSDKLVIYNWGDYIDPALLKKFTKETGIEVQYETFDSNEAMYTKIKQGGTTYDIAVPSDYTIDKMIKENLLNKLDKSKLVGMDNIGKEFLGKSFDPQNDYSLPYFWGTVGIVYNDQLVDKAPMHWEDLWRPEYKNSIMLIDGAREMLGVGLTTFGYSVNSKNLEQLQAAERKLQQLTPNVKAIVADEMKGYMIQGDAAIGITFSGEASEMLDSNEHLHYIVPSEGSNLWFDNLVLPKTMKHEKEAYAFLNFINRPENAAQNAAYIGYATPNKKAKALLPDEIKNDPAFYPTDDIIKKLEVYDNLGSRWLGIYNDLYLQFKMYRK

SEQ ID NO: 28 - DNA, 1074 bp, Streptococcus pyogenes
atgcgtaaactttattcctttctagcaggagttttgggtgttattgttattttaacaagtctttctttcatcttgcagaaaaaatcgggttctggtagtcaatcggataaattagttatttataactggggagattacattgatccagctttgctcaaaaaattcaccaaagaaacgggcattgaagtgcagtatgaaactttcgattccaatgaagccatgtacactaaaatcaagcagggcggaaccacttacgacattgctgttcctagtgattacaccattgataaaatgatcaaagaaaacctactcaataagcttgataagtcaaaattagttggcatggataatatcgggaaagaatttttagggaaaagctttgacccacaaaacgactattctttgccttatttctggggaaccgttgggattgtttataatgatcaattagttgataaggcgcctatgcactgggaagatctgtggcgtccagaatataaaaatagtattatgctgattgatggagcgcgtgaaatgctaggggttggtttaacaacttttggttatagtgtgaattctaaaaatctagagcagttgcaggcagccgagagaaaactgcagcagttgacgccgaatgttaaagccattgtagcagatgagatgaaaggctacatgattcaaggtgacgctgctattggaattaccttttctggtgaagccagtgagatgttagatagtaacgaacaccttcactacatcgtgccttcagaagggtctaacctttggtttgataatttggtactaccaaaaaccatgaaacacgaaaaagaagcttatgcttttttgaactttatcaatcgtcctgaaaatgctgcgcaaaatgctgcatatattggttatgcgacaccaaataaaaaagccaaggccttacttccagatgagataaaaaatgatcctgctttttatccaacagatgacattatcaaaaaattggaagtttatgacaatttagggtcaagatggttggggatttataatgatttatacctccaatttaaaatgtatcgcaaataa

SEQ ID NO: 29 - protein, 115 aa, Streptococcus pyogenes
MAFEKEIKLKDCKYLYTISPNIKKYTLRDTTFSQTKVGHYQLIRLLEKIPNSGDGFPLKITINKELTGFKLAITDQSGLRLVNIFKTKDNQILQDKFYFLMDSLVEREIFSKTVV

SEQ ID NO: 30 - DNA, 348 bp, Streptococcus pyogenes
atggcttttgaaaaagaaattaagcttaaagactgtaaatacctttatacaatcagtccaaacattaaaaaatataccctaagagatactacatttagtcaaactaaagtagggcactaccaattaatacgtttactcgaaaaaatacctaattcaggggatggtttccctttaaaaatcactattaataaagaactcacagggtttaaattagctatcaccgatcagtctggtcttagattagttaatatttttaaaacaaaagataatcaaatccttcaagataaattttatttcttgatggacagccttgtagaacgtgaaatttttagcaaaacagtcgtctaa

SEQ ID NO: 31 - protein, 375 aa, Streptococcus pyogenes
MTYFDNAATTPLSPNVIRAMTAAMQDNFGNPSSIHFYGRRANKILRECRQAIARNLGASEQQIIVTSGGTESNNMAIKGYALAHQAKGKHLITTTIEHHSVLHTMAYLEERFGFEVTYLPCQNGQINLSDLKQALRDDTILVSIMYANNETGDLLPIKDIGNLLKDHQAAFHVDAVQAVGKLKIIPSELGIDFLSASAHKFHGPKGCGFLYSNGQPIDPLLHGGDQEGKRRASTENMLGIIGMAQALTDAMTCLDQSTDHIISLRHHLISLLEGLPYYINQGTHYLPHVLNIGFLGYQNTILLTQLDLAGIAVSTGSACTAGAVNPSHVLAAYYGDDSSRLKESIRISFSDQNSIEDVNQLAQTLKNILGGTDGF

SEQ ID NO: 32 - DNA, 1128 bp, Streptococcus pyogenes
atgacttactttgataacgccgccaccacaccacttagtcctaatgtgattagggcgatgacagcagctatgcaagataactttggtaacccctctagtattcatttctacggtcgccgagccaataaaatccttcgtgaatgccgacaagctatcgctagaaacttgggggcaagtgaacagcaaattatagtaacgtccggtggaactgaaagcaataatatggccataaaaggctatgctttagctcatcaagcaaagggcaaacatctcatcacaactactattgagcaccactctgtgcttcataccatggcctatctcgaagagcgatttggttttgaggtcacttatttgccttgtcaaaatggacaaataaatttatctgacttaaaacaagccttgagagatgacaccattttggtaagcataatgtatgccaataacgaaacaggtgacctgttacctattaaggacattgggaatctgctcaaagaccatcaagctgcttttcatgttgatgccgttcaagctgttggcaaactcaagattattcctagcgaacttgggattgactttttatctgcttctgctcacaaatttcatggtcctaaaggatgcggctttttatacagtaacggacaaccaatcgaccctcttcttcacgggggtgatcaagaaggaaagcgacgcgcaagtactgaaaatatgttaggtattattggtatggcacaagctttgactgatgctatgacttgtcttgaccaatcaactgatcacattattagtttacgtcatcacttgattagcctcctagaaggactaccctactatattaatcaaggtactcattatcttcctcatgttctcaatattggatttttaggctaccaaaatactattttactaacacaacttgacttagcaggcattgcagtatcaactggttctgcttgtactgctggagctgttaatccaagtcatgttttagctgcgtactatggggatgactcttctcgtttaaaagaatcaattcgtatcagtttttcagatcaaaatagcattgaagatgttaatcaattagctcaaacattaaaaaatattttaggaggaacagatggcttttga

SEQ ID NO: 33 - protein, 575 aa, Streptococcus pyogenes
MIKTDHHLLKRVLQDLLKKPLPVCILVIASFVQVGLSVYLPVLIGKAVDMSLSVNSWQTLKWLLGQMLVIIVVNTLIQWVMPLVYSRLLYQYSQQLKDKLLEKIHRLPFAYLDRQTIGDLVSRVITDTEQLINGLQMVFNQFILGLLTILCTIIAMAQIDWLMLILVLVLTPSSLFLARFIAQKSFHYAQAQTKSRGNLAQFTEEILRQEGLVQLFNAQEQSICDYHVLNKTYCEASQKAIFYASTVNPATRFINSVIYALLAGLGAVRIMAGLFSVGQLTTFLNVVVQYTKPFNDISSVLAEIQSSLACAQRLYDLLDIEIKEQEHFLTFKASAVKGQIDFEEVSFSYQKDRPLLKDINFSVPAGSKVAIVGPTGAGKSTLINLLMRFYELDAGSIKLDKVPIKCYAKEELRSITGIVLQETWLKDATVHELIAYGSEEASRDEVVAAAKAAHAHFFIMQLPKTYDTYLSASDDALSQGQLQLLAIARMFLKKPKVLVLDEATSSIDIRTEAVIQEALKELMRGRTSFIIAHRLSTIQSADLILVMDQGRLVEWGTHASLMSKNGCYVRLQKIE

SEQ ID NO: 34 - DNA, 1728 bp, Streptococcus pyogenes
atgattaaaacagatcatcacctcctaaagcgagtgcttcaggatttgttaaaaaaacctctacctgtttgtattctcgtgatcgctagttttgtacaagttggtttaagtgtttacttgcctgttcttatcggtaaagccgttgatatgagtttgtcagtcaattcttggcaaactttaaaatggctgttggggcaaatgttagttattattgtggtcaacacattgattcaatgggtgatgcctcttgtctatagtcgtttgctatatcaatatagccagcaattgaaagataagcttttggaaaaaattcatcgtcttccttttgcatatctagatcgtcaaaccattggagatttggttagtcgagtgataaccgacactgagcaactgatcaatggtctacaaatggtttttaaccagtttatacttggactattgacgattctgtgtaccattatagctatggcacagattgactggctcatgttaatcttggttttggtgttaacaccaagctccctctttttagctcgttttattgctcaaaagagttttcattatgcacaagcacagaccaaaagcagaggaaatttagctcagtttactgaagaaatccttcgtcaagaaggtttggttcaactgtttaatgctcaagagcagtctatctgtgattatcatgttttaaataaaacctattgcgaggcttcacaaaaagccattttttacgcctcaacagtcaatccagccactcgctttattaatagtgtcatttatgctttactggcaggtttaggggctgttcgtatcatggctggacttttttcagtaggtcagctaaccacttttttaaatgttgttgtccaatataccaaacctttcaacgatatttcatctgtcttggcagagatacagagttctttagcttgtgcgcaaaggctttatgaccttttagatattgaaataaaagagcaagagcactttctaacatttaaggcatcagcggttaaggggcaaattgactttgaagaagtaagtttttcttatcaaaaagacaggcccttactgaaagatattaatttttctgttcctgcgggttccaaggtagcgattgtagggcctacaggtgcaggtaaatcaactttaatcaatttactcatgcgcttttatgaacttgatgctggtagcatcaagttagataaagttcctattaagtgttatgctaaggaagaacttaggtccattactggcatagtattgcaagaaacctggttgaaagatgcgactgttcatgagttgattgcttacggcagtgaagaggctagccgtgatgaagtagtggcagcagccaaagcagctcatgcacacttctttattatgcaacttcctaagacttatgatacttacttaagtgcttctgatgatgctttgtcccaagggcagctccagttattagctattgccagaatgtttttgaaaaaaccaaaagtcttggttctagatgaagccacctcctctattgatattagaacagaagctgttattcaagaggcactaaaagaactcatgagaggaaggaccagctttatcattgcccatcgtttatcaacgattcaatcagctgatttgattcttgttatggatcaaggtcgattggttgagtggggaacacatgccagcttaatgtcaaaaaacggctgttatgttagattacaaaagatagaataa

SEQ ID NO: 35 - protein, 456 aa, Streptococcus pyogenes
MSKIVVVGANHAGTACIKTMLTNYGDANEIVVFDQNSNISFLGCGMALWIGEQIAGPEGLFYSDKEELESLGAKVYMESPVQSIDYDAKTVTALVDGKNHVETYDKLIFATGSQPILPPIKGAEIKEGSLEFEATLENLQFVKLYQNSADVIAKLENKDIKRVAVVGAGYIGVELAEAFQRKGKEVVLIDVVDTCLAGYYDRDLTDLMAKNMEEHGIQLAFGETVKEVAGNGKVEKIITDKNEYDVDMVILAVGFRPNTTLGNGKIDLFRNGAFLVNKRQETSIPGVYAIGDCATIYDNATRDTNYIALASNAVRTGIVAAHNACGTDLEGIGVQGSNGISIYGLHMVSTGLTLEKAKRLGFDAAVTEYTDNQKPEFIEHGNFPVTIKIVYDKDSRRILGAQMAAREDVSMGIHMFSLAIQEGVTIEKLALTDIFFLPHFNKPYNYITMAALGAKD

SEQ ID NO: 36 - DNA, 1371 bp, Streptococcus pyogenes
atgagtaaaatcgttgttgttggtgcaaaccatgctggtacggcctgtatcaagactatgttaacaaactacggtgatgctaatgagattgttgtatttgaccaaaactcaaatatttcatttttaggctgtggtatggcactttggattggtgagcaaattgctggaccagaaggacttttctattcagataaagaagaattagagtctttaggggctaaagtttacatggaatcacctgttcaatcaatcgactacgatgccaaaacagtgacagcgcttgttgatggtaaaaaccacgtggaaacttacgacaagttgatttttgcaactggctcacaacctatcttaccaccgattaaaggtgccgaaatcaaagaaggatcacttgaatttgaagcaactcttgaaaatcttcaatttgtgaagttataccaaaactcagctgatgtgattgcaaagcttgaaaacaaagacattaaacgtgtagccgtagttggtgctggttacattggtgttgagttagctgaagctttccaacgcaaaggcaaagaagtggttctgattgatgtcgtggacacttgcttggcaggttattacgaccgtgatttgactgacttaatggctaaaaacatggaagaacatggtattcaactagcctttggtgaaacagttaaagaagtagctggtaatggtaaggttgaaaagatcattactgacaaaaatgagtacgatgtggatatggttatcctcgccgttggtttccgtccaaacacaactcttggaaatggtaagattgatcttttccgtaatggtgctttccttgtcaataaacgccaagaaacttctattccaggtgtttatgctatcggtgactgtgcaactatctacgataatgctactcgcgatacaaactacattgctttagcttcaaatgccgtccgtacaggaattgtagcagcacataacgcttgtggtacagaccttgaaggtattggcgttcaaggctcaaacggtatttccatttacggattgcacatggtttcaactggtttgacacttgaaaaagcaaaacgtcttggttttgatgctgcagtgactgagtatactgataaccaaaaacctgaatttatcgaacacggtaacttcccagtaaccattaagattgtttacgataaagactcacgtcgtatcttgggtgctcaaatggcagcccgtgaagatgtgtcaatgggaattcacatgttctcacttgctatccaagaaggcgtaaccattgaaaagttggcattaactgatattttcttcttaccacatttcaacaaaccttacaactacatcacaatggcagcacttggtgccaaagactag

SEQ ID NO: 37 - protein, 232 aa, Streptococcus pyogenes
MFSGHQLKTARLSKGITQSELGRLLHVNKMTISNWEKGKNIPNEKHLNALLHLFNVTSDYFDPNYRLLTPYNQLTISNKEKVIGYSERLLNHQIDKKSKDLIDKPSQLYAYRVYESLSAGTGYSYFGDGNFDVVFYDEQLEYDFASWVFGDSMEPTYLNGEVVLIKQNSFDYDGAIYAVEWDGQTYIKKVFREDEGLRLVSLNKKYSDKFAPYSEEPRIIGKIIANFRPLEI

SEQ ID NO: 38 - DNA, 699 bp, Streptococcus pyogenes
atgttttctggacatcaactaaaaacagcacgattatcaaagggaataactcaatcagaattgggaagattgttgcatgtcaataaaatgacaatatctaattgggaaaaaggtaagaatataccaaatgaaaaacatttaaatgccttattgcatctattcaatgtgacatctgattatttcgacccaaactatagattgctaacgccttataaccagctgacaatatctaataaagaaaaagtaattggctattcagagcgattgttaaatcatcaaatagacaaaaaatctaaagatctcatagataaaccatcacaattatatgcttatcgggtctatgaaagtttatctgctggtactggttactcctattttggtgatggtaactttgatgttgtcttttacgatgaacaattagaatacgattttgcgtcttgggtttttggagattctatggagccaacttatttaaatggtgaagttgttcttataaaacaaaatagttttgattacgatggagcaatttatgcagtcgaatgggatgggcaaacatatatcaaaaaggtatttcgtgaagatgagggattacgtctagtgtccttaaataaaaaatattctgataagtttgctccctatagcgaagaacctcgcattattggcaaaattatcgctaattttaggcccttagaaatttaa

SEQ ID NO: 39 - protein, 306 aa, Streptococcus pyogenes
MSNEFINFEKISRESWKTLHQKAKALLTQEELKSITSLNDNISINDVIDIYLPLINLIQVYKIAQENLSFSKSLFLKKDIQLRPFIIGISGSVAVGKSTTSRLLQLLLSRTHPNSQVELVTTDGFLYPNQFLIEQGLLNRKGFPESYNMELLLDFLDSIKNGQTAFAPVYSHDIYDIIPNQKQSFNNPDFLIVEGINVFQNQQNNRLYMSDYFDFSIYIDADSSHIETWYIERFLSILKLAKRDPHNYYAQYAQLPRSEAIAFARNVWKTVNLENLEKFIEPTRNRAELILHKSADHKIDEIYLKK

SEQ ID NO: 40 - DNA, 921 bp, Streptococcus pyogenes
atgtcaaatgaatttattaactttgaaaaaatttctagagagtcttggaaaacattacatcaaaaggcaaaagctctcctcacacaagaggaacttaaaagtattactagcttaaatgataacatcagtattaacgatgtcattgatatctaccttccattaattaatctgatccaggtttacaaaattgcgcaagaaaatctttctttttctaaaagcctttttctaaaaaaagacattcaattaaggccttttattattggaatttctggttcagtagctgttggtaaatcaacaactagtcgtcttttacaacttttattatcacgaacgcaccccaatagtcaagtcgaactggtaacgactgatggttttttatatcctaatcagtttcttattgaacaaggactgttaaatcgtaaaggatttccagaatcttataacatggaacttctcttagattttctagattccatcaaaaatggtcaaactgcttttgcacccgtctattctcatgatatttacgatattatcccaaatcaaaaacaatcatttaataaccctgacttccttatcgtcgaaggtattaacgtttttcaaaatcaacaaaacaatcgtttatatatgagtgattactttgatttttctatttatattgatgcagacagcagtcacatcgaaacctggtacattgaacggtttctaagcattttaaaattagcaaaacgtgaccctcataattattatgcccaatatgcacaattacctcgatctgaggctatagcatttgcgcgcaacgtttggaaaaccgttaatttagaaaatcttgaaaaattcattgaacctacacgtaaccgtgctgaattaattttacataaaagtgctgatcataaaattgatgaaatttacctcaaaaagtga

SEQ ID NO: 41 - protein, 1036 aa, Streptococcus pyogenes
MFAQLDTKTVYSFMDSLIDLNHYFERAKQFGYHTIGIMDKDNLYGAYHFIKGCQKNGLQPVLGLEIEILYQERQVLLNLIAQNTQGYHQLLKISTAKMSGKLHMDYFCQHLEGIAVIIPSKGWSDTLVVPFDYYIGVDQYTDLSHMDSKRQLIPLRTVRYFAQDDMETLHMLHAIRDNLSLAETPVVESDQELADCQQLTAFYQTHCPQALQNLEDLVSGIYYDFDTNLKLPHFNRDKSAKQELQDLTEAGLKEKGLWKEPYQSRLLHELVIISDMGFDDYFLIVWDLLRFGRSKGYYMGMGRGSAAGSLVAYALNITGIDPVQHDLLFERFLNKERYSMPDIDIDLPDIYRSEFLRYVRNRYGSDHSAQIVTFSTFGPKQAIRDVFKRFGVPEYELTNLTKKIGFKDSLATVYEKSISFRQVINSRTEFQKAFAIAKRIEGNPRQTSIHAAGIVMSDDALTNHIPLKSGDDMMITQYDAHAVEANGLLKMDFLGLRNLTFVQKMQEKVAKDYGCQIDITAIDLEDPQTLALFAKGDTKGIFQFEQNGAINLLKRIKPQRFEEIVATTSLNRPGASDYTTNFIKRREGQEKIDLIDPVIAPILEPTYGIMLYQEQVMQIAQVYAGFTLGKADLLRRAMSKKNLQEMQKMEEDFIASAKHLGRAEETARGLFKRMEKFAGYGFNRSHAFAYSALAFQLAYFKAHYPAVFYDIMMNYSSSDYITDALESDFQVAQVTINSIPYTDKIEASKIYMGLKNIKGLPRDFAYWIIEQRPFNSVEDFLTRTPEKYQKKVFLEPLIKIGLFDCFEPNRKKILDNLDGLLVFVNELGSLFSDSSFSWVDTKDYSVTEKYSLEQEIVGVGMSKHPLIDIAEKSTQTFTPISQLVKESEAVVLIQIDSIRIIRTKTSGQQMAFLSVNDTKKKLDVTLFPQEYAIYKDQLKEGEFYYLKGRIKERDHRLQMVCQQVQMAISQKYWLLVENHQFDSQISEILGAFPGTTPVVIHYQKNKETIALTKIQVHVTENLKEKLRPFVLKTVFR

SEQ ID NO: 42 - DNA, 3111 bp, Streptococcus pyogenes
atgtttgctcaacttgatactaaaactgtatactcatttatggatagtttaattgacttaaatcattattttgaacgagcaaagcaatttggttaccacaccataggaatcatggataaggataatctttatggtgcttaccattttattaaaggttgtcaaaaaaatggactgcagccagttttaggtttggaaatagagattctctatcaagagcggcaggtgctccttaacttaatcgcccagaatacacaaggctatcatcagcttttaaaaatttccacggcaaaaatgtctggcaagcttcatatggattacttctgccaacatttggaagggatagcggttattattcctagtaagggttggagcgatacattagtggtcccttttgactactatatcggtgttgatcagtatactgatttatctcatatggattctaagaggcagcttatacccctaaggacagttcgttattttgcgcaagatgatatggaaaccctgcacatgttgcatgccattcgagataacctcagtctggcagagacccctgtggtagaaagtgatcaagagttagcagattgtcaacaactaaccgccttctatcaaacacactgccctcaagctctacagaatttagaagacttagtgtcaggaatctattatgatttcgatacaaatttaaaattgcctcattttaatagagataagtctgccaagcaagaattgcaagacttgactgaggctggtttgaaggaaaaaggattgtggaaagagccttatcaatcgcgcttactacatgaattggtcattatttctgacatgggctttgatgattattttttgattgtgtgggatttacttcgctttggacgcagtaaaggctattatatgggaatgggacgtggctcggcggcaggtagtctagtggcttatgctctgaacattacagggattgatccagttcaacatgatttgctatttgagcgctttttaaacaaagaacgttatagcatgcctgatattgatatcgatcttccagatatttaccgttcagaatttctacggtatgtccgaaatcgttatggtagcgaccattcggcgcaaattgtgaccttttcaacctttggccctaaacaggctattcgtgatgttttcaaacggttcggggttccagaatacgaactgactaatctcactaaaaaaattggttttaaagatagcttggctactgtctatgaaaagtcaatctcttttaggcaggttattaatagtagaactgaatttcaaaaggcttttgccattgccaagcgtatcgaaggaaatccaagacaaacgtccattcacgcagctggtattgtgatgagtgatgatgccttgaccaatcatattcctctaaaatcgggcgatgacatgatgatcacccagtatgatgctcatgcggtcgaagctaatggcctgttaaaaatggattttttggggttaagaaatttgacctttgttcaaaaaatgcaagagaaggttgctaaagactacgggtgtcagattgatattacagccattgatttagaagacccgcaaacgttggcactttttgctaaaggggataccaagggaattttccaatttgaacaaaatggtgctattaatcttttaaaacggattaagccacaacgttttgaagaaattgttgccactaccagtctaaatagaccaggggcaagtgactataccactaatttcattaaacgaagagaaggacaagaaaaaattgatttgattgatcctgtgattgctcccattttagagccaacttacggtattatgctttatcaagaacaagttatgcagattgcacaggtttatgctggttttacgttaggcaaggccgacttgttaaggcgtgccatgtctaaaaaaaatctacaagaaatgcaaaaaatggaagaagactttattgcttctgctaagcacctagggagagctgaagaaacagctagaggactttttaaacggatggaaaaatttgcaggttatggttttaaccgcagccatgcctttgcctattcagctttagcttttcaattggcttatttcaaagcccattacccggctgttttttacgatatcatgatgaattattctagcagtgactatatcacagatgctctagaatcagattttcaagtagcgcaagttaccattaatagtattccttacactgataaaattgaagctagcaagatttacatggggctgaaaaatattaaggggttgccaagggattttgcttattggattatcgagcaaagaccatttaatagcgtagaggattttctcactagaactccagaaaaatatcaaaaaaaggttttccttgagcctctgataaaaataggtctgtttgattgctttgagcctaaccgtaaaaaaattctggacaatttggatggtttactggtatttgttaatgagcttggttctcttttttcagattcttcctttagttgggtagatacgaaagattactcagtaactgaaaaatattctttggaacaggagatcgttggagttggcatgagcaagcatcctttaattgatattgctgagaaaagtacccaaacttttactcctatttcacagttagtcaaagaaagcgaagcagtcgtactgattcaaatagatagcattaggatcattagaaccaaaacaagtgggcagcaaatggcttttttaagtgtgaatgacactaagaaaaagctcgatgtcacactttttccacaagagtatgccatttataaagaccaattaaaagaaggagaattctattacttaaaaggtagaataaaagaaagagaccatcgactgcagatggtgtgtcagcaagtgcaaatggctattagtcaaaaatattggttattagttgaaaaccatcagtttgattcccaaatttctgagattttaggtgcctttccaggaacgactccagttgttattcactatcaaaaaaataaggaaacaattgcattaactaagattcaggttcatgtaacagagaatttaaaggaaaaacttcgtccttttgttctgaaaacggtttttcgataa

SEQ ID NO: 43 - protein, 711 aa, Streptococcus pyogenes
MRELHIKTYKLLTKSAVLLGLISFPLTVSAADNASVTNKADFSTDTIYQIVTDRFNDGNTSNNGKTDVFDKNDLKKYHGGDWQGIIAKIKDGYLTDMGISAIWISSPVENIDSIDPSNGSAAYHGYWAKDFFKTNQHFGTEADFQQLVKVAHQHHIKVVIDFAPNHTSTAEKEGTTFKEDGALYKNGKLVGKFSDDKDKIFNHESWTDFSTYENSIYHSMYGLADLNNINPKVDQYMKEAIDKWLDLGVDGIRVDAVKHMSQGWQKNWLSHIYEKHNVFVFGEWFSGHTDDDYDMTTFANNSGMGLLDFRFANAIRQLYTGFSTFTMRDFYKVLENRDQVTNEVTDQVTFIDNHDMERFATKVANNQTAVNQAYALLLTSRGVPNIYYGTEQYATGDKDPNNRGDMPSFNKESQAYKVISKLAPLRKQNQALAYGTTEQRWISDHVLVFERKFGNHVALVAINRDQTNGYTITNAKTALPQNSYKDKLEGLLGGQELIVGADGTISSFELGAGQVAVWTYEGEDKTPQLGDVDASVGIAGNKITISGQGFGNSKGQVTFGEISAEILSWSDTLITLKVPTVPANYYNISVTTADKQTSNSYQAFEVLTDKQIPVRLLINDFKTVPGEQLYLMGDVFEMGANDAKNAVGPLFNNTQTIAKYPNWFFDTHLPINKEIAVKLVKKDSIGNVLWTSPETYSIKTGHEAQTITIKK

SEQ ID NO: 44 - DNA, 2136 bp, Streptococcus pyogenes
atgagagaattacatatcaaaacttataagttattaacgaaaagtgctgttttacttggcttaatttcatttccactaactgtttctgctgccgataatgcttctgtcaccaacaaagcagatttttcaacagatacgatttatcagattgtaacagatcgttttaacgatggtaatacctctaataatggtaagactgatgtttttgataaaaatgaccttaaaaaataccatggaggtgattggcaaggaatcatcgccaagattaaggatggttacctgacagatatggggatttctgccatttggatttcttctcctgttgaaaatatcgacagtattgatccttctaatggaagtgctgcatatcatgggtattgggctaaggacttctttaaaacaaaccagcattttggcactgaagcagactttcaacaactagtcaaagtagctcatcaacaccatattaaggtagttattgattttgctcctaatcatacgtctacagccgaaaaagaaggcacaactttcaaagaagatggcgctttatataaaaacggtaaattagttggtaaattttcagatgataaagacaagatttttaatcatgaatcttggaccgattttagtacttatgaaaattctatttatcattcaatgtacggactagctgatttaaataacattaatccgaaagttgaccagtacatgaaagaagctattgataaatggttagacctgggtgttgatggtatccgagttgacgctgttaaacatatgtcacaaggttggcaaaaaaattggttgagtcatatctatgaaaaacataatgtctttgttttcggggaatggttctcgggacataccgacgatgattatgatatgacgacatttgctaacaatagtgggatggggcttttagattttagatttgccaatgctattagacagttgtatacaggtttttcaacgtttaccatgcgagatttttacaaggttcttgaaaatagagatcaggtgactaatgaagtgacagaccaggtgacctttattgataatcatgatatggaacgcttcgcaacaaaagtggctaataatcaaactgctgttaatcaagcctatgctttgcttttaacatctagaggtgtgcctaatatttattatggtacagagcagtatgcaacaggtgataaagatcctaataatcgtggtgatatgccaagttttaataaagagtcacaagcctataaagtgattagtaagctagctcctttaagaaaacaaaatcaagctttagcttatggaacaactgaacaacgttggattagtgatcatgttttggtatttgagcgtaaatttggtaatcatgtcgcactagtggctattaatagagatcaaacgaatggttatacaattactaatgctaaaacagccttgccccaaaatagctacaaggacaaattagaaggtcttcttggcggtcaagaattaatagttggagcagatggcactattagtagctttgaacttggagcggggcaagtcgctgtatggacttatgaaggagaggacaagacaccacaacttggagatgtcgatgcttcagtgggtattgctggaaataagattactatttcaggtcaaggttttggtaattctaaaggtcaagtgacttttggagaaatctctgctgagatcctttcttggtcagatacccttatcaccttaaaagtaccgacggttccagcaaattattataacatttcagtgacaactgccgataagcaaaccagcaatagttaccaagcctttgaagtattgactgataaacaaattcctgttcgtttactcatcaatgattttaagacagtaccaggggaacaactatatctcatgggtgatgtttttgagatgggggcaaatgacgctaagaatgctgttggtcctctatttaataacactcagaccattgccaagtacccaaactggttctttgatactcatctaccaatcaataaagaaatagcagtcaaacttgttaaaaaagatagtattgggaatgttttatggacaagtcctgagacttatagtataaagacaggtcatgaagcacaaaccattactataaaaaaataa

SEQ ID NO: 45 - protein, 427 aa, Streptococcus pyogenes
MSKHQDILDYLEKLAIGKKVSVRSISNHLKVSDGTAYRAIKEAENRGIVETKPRSGTVRIEKKGRVRIDRLTYSEIARISDSEVLAGHAGLGHEFSRFSIGAMTQQNIRRYLVKGGLLIVGDRETIQLLALENHNAILVTGGFPVSKRVIEMANNQRIPVMVTHYDTFTVATMINHALSNIRIKTDLKTVEQVMIPITDYGYLCEDSSVEEFNTLIKKTRQVRFPVLDYKRKVIGVVSMRDVVDQLPTTKLTKVMSKNPITARPNTSLANISQKMIFEDLNMLPVTDEENNLLGMITRRQAMENLPNHQPNNPYTYSEQILSNLEETVDYYQVVVEPTMIDSAGNMSNGVISEFLKEISIRALTKKHQKNIIIEQMMVYFLHAIQIEDELKIYPKIITENRRSSTIDIEIFVDDQVIAKAIITTKIN

SEQ ID NO: 46 - DNA, 1284 bp, Streptococcus pyogenes
atgagcaaacatcaagatattttagattatttagaaaaacttgctattggtaaaaaagtgagtgttagaagcatttcaaaccacttaaaagtaagcgatggaacagcctaccgtgctattaaagaggcagaaaatcgaggtattgtcgaaaccaaaccaagaagtggaactgttcgtattgaaaaaaaaggacgggttcgtattgaccgtctgacctattccgaaattgctcggatcagtgattcggaagtgctagcgggacatgctggtttaggacatgaatttagtaggttttcaattggtgctatgactcaacaaaatattcgtcgttacttggtcaaaggagggttattaatagttggggatcgtgaaacgattcaactcttagctcttgaaaaccataatgccattttggtgacgggcggatttccagtatccaaacgtgttattgaaatggcaaataaccaacgaatcccagttatggtaactcattatgatacctttactgtggcaacaatgattaatcacgcgctatctaatattcgtattaaaacagatctcaaaacagttgaacaggtaatgatacctattacagattatggttacctttgcgaagacagctctgttgaagaattcaatacattaataaaaaaaacacgacaagtcaggtttcctgtattagattataaaagaaaagtgattggtgtagttagtatgcgtgatgtggtggatcagttgccaacaactaagcttaccaaggtcatgtctaaaaatccaataacggctagaccaaatactagtcttgctaatattagccagaaaatgatctttgaagatttaaacatgttacctgtcactgacgaagaaaacaatttacttggtatgattactcgtagacaagcaatggaaaatttgccaaatcaccaaccaaataacccctatacttatagtgagcaaatactttctaaccttgaagaaacggttgattattatcaagtagtagttgagccaacaatgattgatagtgctggtaacatgtcaaacggtgttatttcagagttcttaaaggaaattagtatcagagccctcacgaaaaagcaccaaaaaaatatcattatcgaacaaatgatggtatactttttacatgctattcaaattgaggatgaattaaaaatatatcctaaaatcattactgaaaatagacgaagcagcaccattgatattgaaatttttgtggatgatcaagtgattgctaaagctattattacaacgaaaataaattag

SEQ ID NO: 47 - protein, 180 aa, Streptococcus pyogenes
MDIWTKLAVFAFFETPKVILRPFRYEDHWDFYSMVNDTKNLYYVFPEQKTKAASDYLLVHSFIKFPLGQWAIEDKATHQVIGSIRIEHYDAKTRCADIGYFLNYAFWGQGIMTEVVIKLVYLSFHEFGLKTLRIITHLENKASQKVAKKAGFQLKTCFKGSDRNTHKICIYKMYQLTNDR

SEQ ID NO: 48 - DNA, 543 bp, Streptococcus pyogenes
atggatatttggacaaagcttgcagtgtttgctttttttgagactccgaaagtaatattacggccttttcgctatgaagatcattgggatttttacagcatggttaacgacactaaaaacctttattatgtttttccagaacaaaaaactaaggcagcaagtgactatcttttagtacatagttttataaagtttcctttaggtcagtgggcaatagaagataaagcaacccaccaagtaataggttctattagaattgagcattatgatgctaaaacgcgttgtgctgatattggctattttttaaactatgccttttggggacaaggaattatgacagaagtcgtaataaaacttgtttatttatcttttcacgaatttggtctcaaaacgttgcgtattataactcatttagaaaataaggccagccagaaagtagctaaaaaagcaggttttcaactaaaaacctgttttaaaggaagtgatcgtaatactcataaaatctgcatttataaaatgtaccaactaactaatgataggtga

SEQ ID NO: 49 - protein, 721 aa, Streptococcus pyogenes
MITIKNPKILKWLKYVLSAILSLIILVIIIGGLLFTFYISSAPKLSEAQLKSTNSSLVYDGNNNLIADLGSEKRENVTADSIPINLVNAITSIEDKRFFNHRGVDLYRIFGAAFHNLTSQTTQGGSTLDQQLIKLAYFSTNESDQTLKRKAQEVWLALQMERKYTKQEILTFYINKVYMGNGNYGMLTAAKSYYGKDLKDLSYAQLALLAGIPQAPSQYDPYLHPEAAQNRRNVVLQQMYMEKHLTKAEYETAIATPVAEGLQSLQQRSTYPKYMDNYLKQVIEEVKKETNKDIFTAGLKVYTNIIPDAQQTLYNIYHSGDYVYYPDQDFQVASTIVDVTNGHVIAQLGGRNQDENVSFGTNQAVLTDRDWGSTMKPITAYAPAIESGVYTSTAQSTNDSVYYWPGTTTQLFNWDLRYNGWMTIQAAIMLSRNVPAVRALEAAGLDYARSFLSSLGINYPEMHYSNAISSNNSSSDKKYGASSEKMAAAYAAFANGGIYHKPRYVNKVEFSDGTSKTFDEKGKRAMKETTAYMMTDMLKTVLTYGTGTAAAIPGVAQAGKTGTSNYTDEELAKIGEKYGLYPDYVGTLAPDENFVGFTKRYAMAVWTGYKNRLTPVYGSSLEIASDVYRSMMTYLTNGYSEDWTMPNGLYRSGGFLYLSGTYASNTDYTNSVYNNLYSNNTTTASSQTTSDDTSSSNDTSNSTNTDNNGSHPSTDDKKTTH

SEQ ID NO: 50 - DNA, 2166 bp, Streptococcus pyogenes
gtgattacaattaaaaatccaaaaatccttaagtggctaaagtatgtattaagtgcaattcttagccttattatccttgttattattattggtggtcttttgtttaccttctacattagcagtgctccgaaactgtcagaagcccagttaaaatcaacaaactctagcttggtttatgacggtaataacaatctgattgctgatttgggttctgaaaagcgtgaaaatgtaacagctgatagtatccctattaatctagttaatgctattacctcaattgaagataaacgtttctttaaccatcgtggagtagatctttatcgtatttttggtgctgcctttcataatctaacgagtcagaccactcaaggggggtcaacgcttgatcagcaactcattaaactagcctatttttctactaatgaatctgatcaaaccttaaaacgtaaggctcaagaagtttggcttgctcttcaaatggagcgaaaatatactaaacaagaaatcctgactttttacatcaacaaagtatatatgggtaatggcaactatggtatgctgacagccgctaagtcttattatggcaaggatcttaaggatttatcttatgcccaactagccctattggctggaatccctcaagctcctagtcaatatgatccttaccttcatcctgaagctgctcaaaatcgccgtaacgtcgtgttgcaacagatgtacatggaaaaacatctgacgaaagcagaatatgaaactgccatcgcaactcccgtcgctgaaggtctacaatcactccaacagcgctcaacttatccaaaatatatggataattatctaaaacaagttattgaagaagtcaaaaaagaaacgaataaagatatttttaccgctggtttaaaagtttataccaatattatccccgatgcgcagcagactctttataatatttatcattctggtgattatgtttactatccagaccaagatttccaagttgcttcaacgattgttgatgtgacaaatggtcatgttattgctcagcttggcggacgtaatcaagatgaaaatgtttcatttgggactaaccaagctgttttaactgatcgtgactggggttctaccatgaagccaatcacagcctatgctcctgctattgaatctggtgtttatacttctactgctcagtcgactaatgactcagtctattattggcctggaaccactacccaattgtttaactgggaccttagatataacggatggatgacaatccaagctgctattatgctatcgcgaaatgtcccagcagtccgagcactggaagccgcaggacttgactatgctcgatctttcttaagcagtttaggtattaactatcccgaaatgcactactcaaacgctatctcaagtaataacagtagctcagataaaaaatatggtgcaagtagtgaaaaaatggccgctgcatacgctgcttttgcaaatggtggtatttatcataaaccaaggtatgtcaataaagtggaatttagtgatggtacaagtaaaacttttgatgaaaaaggaaaacgtgccatgaaagaaaccacggcctatatgatgacagatatgttaaaaactgttctcacttatggtacaggtactgctgctgccattcctggtgttgcgcaagctggtaaaacagggacttctaactacactgatgaggaactagctaaaattggtgaaaaatacggcctttatccagattatgttggtacattagcgccagacgaaaactttgttggctttactaagcgctacgccatggctgtttggacaggttacaaaaaccgcttgaccccagtatacggatcaagtctagagattgcatctgacgtttatcgtagcatgatgacttacttaacaaatggttacagtgaagattggacgatgccaaatggtctttatcgcagtggtggattcctctacttaagcggaacctatgcgagcaacaccgactatactaattcggtttacaacaatctttacagcaataacacgacaacagcttctagccaaacgacttcagatgatactagtagtagcaatgatacaagtaattcaaccaatacagacaacaatggcagtcatccatctaccgatgataaaaagacaactcattaa

SEQ ID NO: 51 - protein, 244 aa, Streptococcus pyogenes
MIEFKHVSKLYGDKEALSDLNVTINDGEIFGLIGHNGAGKTTTISILTSIIEASYGEVFVDGQLLTENREAIKKQIAYVPDSPDIFLNLTPNEYWQFLAKIYGVSDEDREERLAQLTTLFELKEEVNQTIDSFSHGMRQKVIVIGALVSNPNIWILDEPLTGLDPQASFDLKEMMKAHAASGHTVLFSTHVLSVAEQLCDRIGILKKGKLIFVGTIDELKEHHPDKDLESIYLELAGRKAQEEG

SEQ ID NO: 52 - DNA, 735 bp, Streptococcus pyogenes
atgatagaatttaaacatgtttcaaagttgtatggcgacaaagaagctctcagcgatttaaacgtcaccatcaatgacggtgaaatttttggccttatcggacataatggtgcaggtaaaaccacgaccatcagcattttgacttctattatcgaagcgagttatggcgaagtatttgtggatggtcaacttctgacagaaaatagagaagctatcaaaaaacaaattgcttatgtgccagattctcctgatattttcttgaatctaactccaaatgagtattggcagtttttagctaaaatttatggggtgtcagatgaggacagagaagaacggttagcacagctgacaacactttttgaactgaaggaagaagttaatcaaaccattgatagtttctcacatgggatgcgccaaaaagtgattgtgattggtgctcttgtatcaaatcctaacatctggattttggatgaaccattaacaggtttggatcctcaggcttcttttgaccttaaagaaatgatgaaagctcatgcagcttctggacatacagtcttattttcgactcacgtgctatcagtcgctgagcagttatgtgatcgtatcgggattctaaaaaaagggaaattgatttttgttggaacgattgatgagttgaaagaacaccatcctgacaaagacttggagagcatctaccttgaactcgctgggcgtaaggcgcaagaagaggggtga

SEQ ID NO: 53 - protein, 424 aa, Streptococcus pyogenes
MKIGKKIVLMFTAIVLTTVLALGVYLTSAYTFSTGELSKTFKDFSTSSNKSDAIKQTRAFSILLMGVDTGSSERASKWEGNSDSMILVTVNPKTKKTTMTSLERDTLTTLSGPKNNEMNGVEAKLNAAYAAGGAQMAIMTVQDLLNITIDNYVQINMQGLIDLVNAVGGITVTNEFDFPISIAENEPEYQATVAPGTHKINGEQALVYARMRYDDPEGDYGRQKRQREVIQKVLKKILALDSISSYRKILSAVSSNMQTNIEISSRTIPSLLGYRDALRTIKTYQLKGEDATLSDGGSYQIVTSNHLLEIQNRIRTELGLHKVNQLKTNATVYENLYGSTKSQTVNNNYDSSGQAPSYSDSHSSYANYSSGVDTGQSASTDQDSTASSHRPATPSSSSDALAADESSSSGSGSLVPPANINPQT

SEQ ID NO: 54 - DNA, 1275 bp, Streptococcus pyogenes
atgaaaattggaaaaaaaatagttttaatgttcacagctattgtgttaacaactgtcttggcattaggtgtctatctaactagtgcttataccttctcaacaggagaattatcaaagacctttaaagatttttcgacatcttcaaacaaaagtgatgccattaaacaaacaagagctttttctatcttgttgatgggtgttgatacaggctcttcagagcgtgcctccaagtgggaaggaaacagtgattcgatgattttggttacggttaatccaaagaccaagaaaacaactatgactagtttagaacgagataccttaaccacgttatctggacccaaaaataatgaaatgaatggtgttgaagctaagcttaacgctgcttatgcagcaggtggcgctcagatggctattatgaccgtgcaagatcttttgaatatcaccattgataactatgttcaaattaatatgcaaggccttattgatcttgtgaatgcagttggagggattacagttacaaatgagtttgattttcctatctcgattgctgaaaacgaacctgaatatcaagctactgttgcgcctggaacacacaaaattaacggtgaacaagctttggtttatgctcgtatgcgttatgatgatcctgagggagattatggtcgacaaaagcgtcaacgtgaagtcattcaaaaggtattgaaaaaaatccttgctcttgatagcattagctcttatcggaagattttatctgctgtaagtagtaatatgcaaacgaatatcgaaatctcttctcgcactatccctagtctattaggttatcgtgacgcacttagaactattaagacttatcaactaaaaggagaagatgccactttatcagatggtggatcataccaaattgttacctctaatcatttgttagaaatccaaaatcgtatccgaacagaattaggacttcataaggttaatcaattaaaaacaaatgctactgtttatgaaaatttgtatgggtcaactaagtctcagacagtaaacaacaactatgactcttcaggccaggctccatcttattctgatagtcatagctcttacgctaattattcaagtggagtagataccggccagagtgctagtacagaccaggactctactgcttcaagccataggccagctacgccgtcttcttcatcagatgctttagcagctgatgagtctagctcatcagggtctggatcattagttcctcctgctaatatcaaccctcagacctaa

SEQ ID NO: 55 - protein, 300 aa, Streptococcus pyogenes
MTQQSMLLSIEQVNKSYGKNQVLSDISFDIYKGEICGLVGQNGAGKTTLMRILSGLIGKDSGQIKQLQPYRMGSIIESPTLYPNMTAHDNLYYAALQLRLADAKERIHEVLELIGLEKVSKKKKVKDYSLGMRQRLAIGLSILDFPEFLILDEPINGLDPAGIKEMRQIILNLRDCYGITILISSHILSELDLVVDRYVIMHKGKVIKSMDKAELKAQVKVQIALHTSDDQLVKNRLLELGLRVETDGQMLLIKPTLSVMELIKIVLDLPVEIFDIYHHQVSFEHYYLDLLGKDAASPLI

SEQ ID NO: 56 - DNA, 903 bp, Streptococcus pyogenes
atgacacagcaatcaatgttactaagtattgagcaagtaaataaatcctatggtaaaaatcaggttttaagtgatatctcttttgatatttacaaaggagaaatttgcggccttgtgggtcaaaatggtgctggaaagacaacgttaatgcgtattttatctggcctaattggaaaagattcagggcaaatcaaacagttacaaccctatcgaatgggatcaattattgagtcaccgacactttatcccaatatgacagctcatgataatctttactatgcagcgttgcaattgagattagctgatgcaaaagagcgcatacatgaggttttggaacttattgggttagaaaaagtgagcaaaaagaaaaaggtgaaagattattctttagggatgcgccaacgtttggctattggtctttcgatactggattttccagaatttttaatcttggatgaacccattaatggtcttgatccagcaggaatcaaagaaatgcgtcagattattttgaacctaagagattgctatggtattacgattttgatttccagtcatattttatcagaattagacttagttgttgatcgttacgtgattatgcataaaggcaaagttattaagagtatggacaaagcagagcttaaagctcaagtaaaagtacaaattgcccttcatacaagcgatgatcagctggtaaaaaatagacttttagaattggggcttagagttgagactgatggtcagatgctactaataaaaccaactttatcagtaatggagcttatcaaaattgttctagatctgcctgttgagatttttgacatttatcatcatcaagtgagctttgaacattattacctcgatttgttaggtaaagatgcagcatcaccattaatttag

SEQ ID NO: 57 - protein, 294 aa, Streptococcus pyogenes
MIKRCKGIGLALMAFFLVACVNQHPKTAKETEQQRIVATSVAVVDICDRLNLDLVGVCDSKLYTLPKRYDAVKRVGLPMNPDIELIASLKPTWILSPNSLQEDLEPKYQKLDTEYGFLNLRSVEGMYQSIDDLGNLFQRQQEAKELRQQYQDYYRAFQAKRKGKKKPKVLILMGLPGSYLVATNQSYVGNLLDLAGGENVYQSDEKEFLSANPEDMLAKEPDLILRTAHAIPDKVKVMFDKEFAENDIWKHFTAVKEGKVYDLDNTLFGMSAKLNYPEALDTLTQLFDHVGDHP

SEQ ID NO: 58 - DNA, 885 bp, Streptococcus pyogenes
atgataaaacgatgtaaaggaattggtctagccttaatggccttctttttggtagcttgtgtgaatcagcaccctaaaacggctaaagagactgaacagcagagaattgtagccacttcggttgctgtggttgatatctgtgaccgtttaaatttagacctcgttggggtttgtgatagtaaattatatacccttcctaaacgctatgatgctgttaagcgtgtgggtttacccatgaatcctgatatagagttgattgcttctttgaaaccaacttggattttgagtcccaattctttacaagaagatttggaacccaagtatcaaaaattggatactgagtatggttttttgaacttacgaagtgttgagggcatgtaccagtccattgatgatttagggaaccttttccaacgtcaacaagaagcaaaagaattgcgccagcaataccaggactattatcgtgctttccaagctaaacgtaaggggaagaaaaagcctaaagtgcttattcttatgggcttgccaggtagttatttggtggcgacgaaccaatcttatgtagggaatcttttggacttggcaggtggtgagaatgtttatcagtcagatgagaaagaatttctatcagctaatcctgaagacatgctggctaaggagcctgacttgattttacgaacagctcatgccattccagacaaggtaaaagtgatgtttgacaaagaatttgctgaaaatgatatttggaaacattttacggcagtcaaggaagggaaagtctatgatttggacaataccctgtttggcatgagtgctaaattgaactacccagaagccttggacaccttaacacagctttttgaccacgtgggagatcatccgtaa

SEQ ID NO: 59 - protein, 208 aa, Streptococcus pyogenes
MTKALSGSFLEENMKHLDTYIAFDLEFNTVNDVSHIIQVSAVKYDHHKEVDSFDTYVYTDVPLQSFINGLTGITSDKIAAEPKVEEVMAAFKNFVGELPLIGYNAQKSDLPILAENGLDLRDQYQIDLFDEAYDRRSADLNGIANLRLQTVATFLGIKGRGHNSLEDARMTAVIYKSFLETDTNKAYLSQQEEVTTDNPFAALGDFFD

SEQ ID NO: 60 - DNA, 627 bp, Streptococcus pyogenes
atgacgaaggccctatcagggtcttttttggaggaaaacatgaaacacttagatacttatattgcctttgatttagaatttaacactgtcaatgacgttagtcatatcattcaagtatcagcagtcaaatatgaccaccataaagaagttgacagctttgacacctatgtttacacagatgttcccttacagagctttatcaatggcctaacaggaatcacatctgacaagattgctgcggaaccaaaagtagaggaggttatggctgcgtttaaaaattttgtgggagagttaccactgattggttataatgctcaaaaatctgacctgcctattctagctgaaaatggcttggatttaagggaccaataccagattgacctctttgatgaagcttacgatagacgtagcgcagacttaaatgggattgctaaccttcgtctgcaaacagtggcaacctttttaggcattaagggtcgaggtcataatagcttagaagatgccagaatgacagcagtaatctataagtcttttttagaaacagatacaaacaaagcatatctcagccagcaagaagaggtaaccacagataaccctttcgcagctttgggagactttttcgactaa

SEQ ID NO: 61 - protein, 193 aa, Streptococcus pyogenes
MVLFLIRIFSDSDKEENMGIEKTVSELADILGVSRQAVNNRVKSLPEEDLDKNEKGVTVVKRSGLVKLEEIYKKTIFDDEPISEETKQRELLEILVDEKNTEITRLYEQLKAKDAQLASKDEQMRVKDVQIAEKDKQLDQQQQLTAKAMADKETLKLELEEAKAEANQARLQVEEVQAEVGPKKGFFTRLFAK

SEQ ID NO: 62 - DNA, 582 bp, Streptococcus pyogenes
atggtactatttttaataagaatattcagtgatagtgataaagaggaaaatatgggaattgaaaagacagttagtgaactagctgatattttgggagtgagtcgtcaggccgtcaataatcgtgttaagtctttacccgaagaagatcttgacaaaaatgaaaaaggcgttactgtggttaagcgcagtggtcttgttaagctggaagagatctataaaaaaacaatttttgacgatgagcctatcagtgaagaaacgaaacaacgtgagcttttagagattctcgtggatgagaagaacactgaaatcacgcgcctttatgagcaactcaaagccaaagatgctcaacttgcctcaaaagatgagcaaatgcgtgttaaagacgtgcagatcgctgaaaaagataaacaattggatcagcagcaacaattaactgctaaagccatggctgataaagaaaccctaaaattagaattggaagaagctaaagcagaagccaaccaagctcgcttacaagtcgaagaagttcaggctgaagttggccctaaaaaaggctttttcacccgcttgtttgcgaaataa

SEQ ID NO: 63 - protein, 191 aa, Streptococcus pyogenes
MKLDVFAGQEKSELSMIEVARAILEERGRDNEMYFSDLVNEIQNYLGKSDAGIRHALPFFYTDLNTDGSFIPLGENKWGLRSWYAIDEIDEEIITLEEDEDGAQKRKKKRVNAFMDGDEDAIDYRDDDPEDEDFTEESAEVEYDEEDPDDEKSEVESYDSELNEIIPEDDFEEVDINEEDEEDEEDEEPVL

SEQ ID NO: 64 - DNA, 576 bp, Streptococcus pyogenes
ttgaaattagacgtatttgcaggacaagaaaaaagcgagctttccatgattgaagtggcgcgtgccattttggaagaacgcggtcgcgacaatgagatgtactttagcgatttagtcaatgagattcaaaattacttaggaaaatctgatgcaggtattcgtcatgcattaccatttttctatactgacttaaatacggatggctcatttattcctctaggtgaaaacaaatggggacttcgttcgtggtatgctattgatgaaattgatgaagaaattattactctagaagaagatgaagatggcgcacaaaaacgtaagaaaaaacgcgttaatgctttcatggatggagatgaagacgctattgattatagagatgatgatccagaagatgaagatttcacagaagaatcagctgaggttgaatatgatgaagaagatccagatgatgaaaaatcagaagtggagtcatatgattcagaattgaatgaaattatccctgaagatgattttgaagaagttgatatcaatgaagaagacgaagaagatgaagaagatgaagaaccagttctctaa

SEQ ID NO: 65 - protein, 309 aa, Streptococcus pyogenes
MILTVTLNPAIDVSYPLDELKCDTVNRVVDVTKTPGGKGLNVSRVLNEFGETVKATGCVGGESGDFIINHLPDSILSRFYKISGDTRTCIAILHEGNQTEILEKGPMLSVDEIDGFTHHFKYLLNDVDVVTLSGSLPAGMPDDYYQKLIKIANLNGKKTVLDCSGNALEAVLKGDSKPTVIKPNLEELSQLLGKEMTKDFDALKEVLQDELFDGIEWIIVSLGADGVFAKHKDTFYNVDIPKIKIVSAVGSGDSTVAGIASGLANDEDDRALLTKANVLGMLNAQEKTTGHVNMANYDKLYQSIKVKEV

SEQ ID NO: 66 - DNA, 930 bp, Streptococcus pyogenes
gtgattttaacagttactttaaatccagctattgatgtctcctaccctttggatgagctaaaatgcgatactgtcaatcgagttgtagacgttacaaagacacccggtggtaaaggcttgaatgtttcccgagttcttaatgaattcggtgagacagtcaaagctacaggttgtgtaggtggagaaagcggtgattttatcattaatcatcttcctgattcgattttaagtcgcttctataaaatttcaggtgatactagaacttgtatagccattttacacgaaggtaatcaaacggagattttagaaaaaggaccgatgttaagtgtagatgaaattgatggatttactcatcattttaagtatttactgaatgatgttgacgtagtgacgctctcaggcagtttgcctgctggtatgccagatgattactatcagaagttaatcaagattgccaatcttaatggtaagaaaacggttctggattgttctggaaatgctctagaagcagtactaaaaggtgatagtaaaccaacagttatcaaacctaatcttgaagaactttctcaacttctaggtaaagaaatgacaaaagattttgatgccttaaaagaagtcttacaagatgaactatttgatggaattgaatggataatcgtttctcttggtgctgatggtgtatttgcaaagcacaaggatacgttttataacgttgacattcctaaaatcaaaattgtaagcgctgtaggttcaggggattctacagtggcaggtattgcttcaggtttagcgaatgatgaagatgatcgagctttattaacaaaagcaaatgtcttagggatgttaaatgcgcaagaaaaaacaactgggcatgttaacatggcaaattacgataagttatatcaatctattaaagtcaaagaggtataa

SEQ ID NO: 67 - protein, 224 aa, Streptococcus pyogenes
MLNLKDIRKSYHLGTEEFAILKGIDLEVNEGDFLAIMGPSGSGKSTLMNIIGCLDKPGSGSYAIEGRDVSSLSDNELADLRNQKIGFVFQNFNLMPKLTACQNVELPLTYMNVPKKERRKRALEMLKLVGLEERSEFKPMELSGGQKQRVAIARALVTNPSFILGDEPTGALDTKTSVQIMDLFKQFNDNGKTIIIITHEPEVAALCKKTVILRDGNIEHSDIE

SEQ ID NO: 68 - DNA, 675 bp, Streptococcus pyogenes
ttgttaaaccttaaagatattcgaaaaagctatcatcttggaactgaagaatttgcgattttaaaaggaatcgatttagaagttaacgagggtgactttttagccatcatgggaccatcaggttcgggaaagtcaacattgatgaatatcattgggtgtttagataagcctggctctggctcatatgccattgaaggcagagacgtgtcatccttatctgataatgaacttgctgatttgcgtaatcaaaaaatcggctttgtttttcaaaattttaacctgatgcccaagctaacagcttgtcaaaatgtcgaattacccttgacttatatgaatgttcctaaaaaagagcgtcgcaaacgagccctagagatgttaaagctagtaggactagaagaacgtagtgaatttaaaccgatggagctatctggtgggcaaaaacagcgtgtagcgattgcaagagctttagtcactaacccgagttttatccttggtgatgagccaacaggtgcactagacacaaaaaccagcgtccaaatcatggacctatttaaacaattcaatgataacggcaaaacgattattatcatcacacacgagcctgaagtagctgccctatgcaaaaagacggtgatcctaagagatggtaatatagaacattccgatatagagtaa

SEQ ID NO: 69 - protein, 422 aa, Streptococcus pyogenes
MFQLRKKMTRKQLALLSAGVLTCVVGGSYLIMNHQQQEIVSSVNKVKALTIKEAMEQGKDISLTLAGEVTANNSSKVKIDSSKGEVKEVFVKKGDVVKVGQPLFSYETSQRLTAQSSEFDVQTKANQLQVAKTNAALKWETYNRKVNEINTLKSRYNTAPDESLLEQIRSAEDSVSQALSDAKTADSDVKTAQIELDKANATATTEKGKLEYDTVKSDTAGTIVSLNTDLPNQSKSKKENETFMEIIDKSKMLVKGNISEFDRDKLKIGQKVEVIDRKDNSKKWTGKVTQVGNLKAEEKGQGQGQGGNDQQDNPNQAKFPYVIELDQSDKQPLIGSHTYVNVLNNVPEAGKIVLKETFTMAENGKTYVWKVDKNKVKKQEIKTKPFSKGYVEVTSGLTMQDKIAQPLPGMKDGMEVGSIVKP

SEQ ID NO: 70 - DNA, 1269 bp, Streptococcus pyogenes
atgtttcagttaagaaaaaaaatgacgcgcaaacaattagccttgttgagtgctggagtgttgacctgtgtggttggtggtagctacttgataatgaaccatcaacaacaagaaattgtctctagtgtcaacaaagtaaaagccttaaccataaaagaagccatggaacaaggaaaagatatcagcttgaccttagctggcgaagtaacagctaacaacagcagcaaagtcaaaatcgactcaagtaaaggagaagtcaaagaggtctttgttaaaaaaggcgatgttgtcaaagtaggacaacccttgtttagctatgaaacgtcacagcggttaacggctcaaagttcagaatttgatgttcaaaccaaagccaatcagctccaagttgctaaaaccaatgcagcattgaagtgggaaacctacaatcgcaaggtcaatgaaatcaacaccctaaaatctcgctacaacactgcaccagatgagagcttactagagcaaattcgcagcgcagaagacagtgtatcccaagcactaagcgatgccaaaacagcagatagcgatgtcaaaaccgctcaaatcgaactcgataaagctaatgctactgccacaacggaaaaaggtaaactagagtatgacaccgttaagtcagacaccgcaggaaccattgttagtctaaatactgatttgccaaatcaatcaaaatccaaaaaagaaaatgaaacttttatggaaattatcgacaaatcaaaaatgttagtcaaaggtaacattagtgaatttgaccgtgacaagttaaaaatcggtcaaaaagtcgaagtgattgaccgcaaagacaactctaaaaaatggactggaaaagtaacccaagttggcaacctcaaagcagaggaaaaaggccaaggtcaaggccaaggtggcaatgaccaacaagataatccaaaccaagcaaaattcccttatgttattgaacttgaccaatcagacaagcagccactcattggctcacacacctatgttaatgtgctcaacaatgttccagaagctggcaagatcgtattgaaagaaacctttacaatggcagaaaatggaaaaacctatgtgtggaaagttgataaaaacaaggtcaaaaaacaagaaatcaagactaagcccttctcaaaaggttatgttgaggtaacaagtggcttgactatgcaagataagattgctcagccgcttcctggcatgaaagacggtatggaggtaggaagtattgttaaaccttaa

SEQ ID NO: 71 - protein, 126 aa, Streptococcus pyogenes
MKTIRRYDVNEDRGHTGLVEAGDFYYLNYCVGNVGQDIESQINGAFDEMERRLALVGLTLDAVVQMDCLFRDVWNIPVMEKMIKERFNGRYPARKSIQTEFAHHGGPQGLLFQVDGVAYSKHISMT

SEQ ID NO: 72 - DNA, 381 bp, Streptococcus pyogenes
atgaaaacaattagacgttatgatgttaacgaagatagagggcatacaggccttgtagaagcaggagatttctattatttaaactattgtgtcggcaatgttggacaagacattgaaagtcagattaatggagcctttgacgagatggagcgccgattagcattggtggggctcacattagatgcagttgttcaaatggattgtctatttcgagacgtttggaacatacctgtgatggaaaaaatgattaaagagcgctttaatggcagataccctgctcgaaaatcgattcaaacggaatttgcacatcacggtggaccacaaggactactctttcaagtggatggtgtggcttattcaaagcatatttcgatgacctaa

SEQ ID NO: 73 - protein, 272 aa, Streptococcus pyogenes
MLIPRKIRHFLRTKKHVLINGHSVNWQSCVKYGDQVKLFFDHEDYPEKIIVMGQAEKVTCLYEDEHIIIVNKPEGMKTHGNDPTELALLNHVSAYTGQTCYVVHRLDKETSGAILFAKTPFILPILNRLLEKRDIHREYLALVHGSLDSPRVTYHHPIGRHRHDRRKRVVDPINGKKAITEVTLVKNFHKTASLLTCQLQTGRTHQIRVHLAHQGHVLFGDPLYSNGKKDCARLMLHAYQLRLKHPLTQEDICVQAKSATFDAVLNAQKARK

SEQ ID NO: 74 - DNA, 819 bp, Streptococcus pyogenes
ttgttaattccccgtaaaattaggcattttctgagaactaaaaaacatgttcttattaatggtcattctgttaactggcaaagttgtgttaaatacggtgatcaggtcaaacttttttttgatcacgaagattatcctgaaaagattatcgtgatggggcaggccgaaaaagtaacctgcctttatgaagatgagcacatcattattgttaacaaacctgaagggatgaaaacacacggcaatgatccaacagagttggcgctgctcaaccacgtgtctgcttacacgggacagacctgctatgtggttcatcgtctggataaggaaacaagtggggccattttatttgctaaaacaccttttatcttaccaatccttaatcgtcttttagaaaaacgagatatccatcgagaatatttggcactagttcacggatccttagacagtcctagggtaacttaccatcatccaattggtcgtcatcgacatgaccgcagaaaacgtgttgtcgatccgataaatggcaagaaagctattactgaggtaactctagtcaaaaacttccacaaaacggcaagcttactcacttgtcaattacaaactggacggactcatcaaattcgagtccatttagcccatcaaggtcatgttttatttggtgatcctttgtattccaatggaaaaaaagattgtgctcgactgatgctccatgcttatcaattacgtttgaaacaccctctaacccaagaagacatttgcgtgcaagctaaatcagctacatttgacgcagtcttaaacgctcaaaaggctaggaaataa

SEQ ID NO: 75 - protein, 114 aa, Streptococcus pyogenes
MIISKKVRRQSDSKEYYLLRLYHERGDKSMQSQLTYDLLKKQIAEELFDEFKKLIQEKDLANQWVNQTTLVNEYGYSWQTIKRMESYGLKSFKNGKDKMYCLADVNEIKHLMKQ

SEQ ID NO: 76 - DNA, 345 bp, Streptococcus pyogenes
atgattatatcaaaaaaagtccgacggcaatcggactctaaagaatactatttacttagattataccacgaaaggggtgataaatctatgcaatcacaacttacttacgatttactaaaaaagcagatagcagaagagctttttgatgagtttaagaaactcatacaggaaaaagatttagcgaatcagtgggtcaatcaaacaacgctcgtcaatgaatacggctactcatggcaaactataaaacgcatggagagctatggccttaaatcttttaaaaacggtaaagacaaaatgtattgtcttgctgatgtcaacgaaatcaaacacttaatgaaacaataa

SEQ ID NO: 77 - protein, 429 aa, Streptococcus pyogenes
MTKDLLSELIQKHRSFNLSYSAYKQLYEGDHAILQQKQKEQYKPDNRLVVNFAKYIVDTFNGYFIGVPVQTSHENKQVSNYLELLDGYNDQDDNNAELSKICSIYGHGYELVFNDENAEAGITYLTPLEAFIVYDDSIRQKPLFAVRYFYNKGGVLEGSYSDASNITYFKDGEKGIEIGESEPHPFDGVPMIEYVENEERQSLLASVVTLINAFNKAISEKANDVEYFADAYLKILGAELDDETLKSLRDTRIINLKDTDAQQLTVEFLQKPDADATQEHLLDRLENLIFRTAMVANISDESFGTASGIALRYRLQAMDNLAKTKERKFMSGMNRRYKLIASYPTSKIGPKDWIGIKYKFTRNLPANLLEESQIAGNLAGIVSEETQVGVLSIVENPQKEIERKNSDKSTLISRQAGGLNGQNTTTILE

SEQ ID NO: 78 - DNA, 1290 bp, Streptococcus pyogenes
atgacaaaagatttgttaagcgagttaatccaaaaacacaggtcgtttaaccttagttattcagcgtataagcagctatatgagggtgatcatgctattttgcaacaaaaacaaaaagagcaatacaagcctgataatcgcttggttgttaattttgcaaaatacatcgttgatacatttaacggctattttatcggtgtgccagtccaaacgagccacgagaataaacaagtcagcaattatttagagttattagacggatacaatgatcaagacgataacaacgcagaactatcaaagatttgcagtatttacggacatggctatgaattagtttttaatgacgagaatgcagaggctggaattacttatctgacaccgcttgaagcttttattgtctatgatgattccattaggcaaaagccgttatttgctgtgcgctatttttataataagggcggcgttttggaggggtcttattcagatgctagcaacatcacttatttcaaagacggcgaaaaaggcattgaaattggagagagtgagccacatccgtttgatggcgtgccgatgattgagtacgttgaaaacgaagagcgacaaagcttgctagctagtgttgtgacattgattaatgcgtttaataaagctatctccgaaaaagccaatgacgttgagtatttcgcagacgcttatcttaaaattttgggtgctgagttagacgacgagacattaaagtctcttagagacacccgcattatcaatctaaaagacaccgatgcacagcaattaactgttgagtttttacaaaagccagatgccgatgcaacgcaagaacatttgctcgaccgattagagaacttaatttttaggactgctatggttgctaatatcagtgatgaatcgttcggtacagctagcggcatcgctttacgttatcgtttgcaagctatggataacttagctaagacaaaagagcgtaagtttatgagcgggatgaaccgcagatataagcttattgcaagctatcctacgtctaagataggacctaaagattggattggcattaagtataaatttactcgcaatctaccagcaaatctcttagaggagtctcaaatcgcaggtaacttagcggggattgtctcagaagaaacacaggtcggtgtgctatctattgtggagaacccacaaaaagaaattgaaaggaaaaatagtgacaagtcaactttgattagccgacaagcaggaggtctgaatggacaaaacaccacaacaatattggaatga

SEQ ID NO: 79 - protein, 98 aa, Streptococcus pyogenes
MAIEIFGPEFRKKLLEDLIALNMEAIKIAQTKNAKSIEWITMKRLEKETGWGRTKLTQWREQGKFNFKRSSENGKVLYDLADVNRFLRTSGYEKGETT

SEQ ID NO: 80 - DNA, 297 bp, Streptococcus pyogenes
atggccattgaaatatttggacctgagtttagaaaaaaactgcttgaagatttaatcgctctaaatatggaagcgataaaaatagcgcagaccaaaaacgccaagtctattgaatggataaccatgaagcggctagaaaaagaaactggatgggggcgaactaaattgacccagtggagagaacaagggaaatttaactttaaaaggtcatcagaaaacgggaaagtactatatgacctagcagatgttaatagatttttacggaccagtggatatgaaaaaggagaaacaacatga

SEQ ID NO: 81 - protein, 58 aa, Streptococcus pyogenes
MKKKLDKILIDKGMSKKELSEKTGISYNTIMNIGKKDISFNKVKKIADVLGVSLDEFR

SEQ ID NO: 82 - DNA, 177 bp, Streptococcus pyogenes
atgaagaaaaaactagacaaaattctcattgataaaggaatgagtaaaaaagagttatcggagaaaacaggaattagttacaacaccattatgaatatcggtaaaaaagatatttcgtttaacaaagtgaaaaaaattgccgatgtcttgggcgtcagcttagacgaatttagatag

SEQ ID NO: 83 - protein, 1465 aa, Streptococcus pyogenes
MSDLFAKLMDQIEMPLDMRRSSAFSSADIIEVKVHSVSRLWEFHFAFAAVLPIATYRELHDRLIRTFEAADIKVTFDIQAAQVDYSDDLLQAYYQEAFEHAPCNSASFKSSFSKLKVTYEDDKLIIAAPGFVNNDHFRNNHLPNLVKQLEAFGFGILTIDMVSDQEMTEHLTKNFVSSRQALVKKAVQDNLEAQKSLEAMMPPVEEATPAPKFDYKERAAKRQAGFEKATITPMIEIETEENRIVFEGMVFDVERKTTRTGRHIINFKMTDYTSSFALQKWAKDDEELRKFDMIAKGAWLRVQGNIETNPFTKSLTMNVQQVKEIVRHERKDLMPEGQKRVELHAHTNMSTMDALPTVESLIDTAAKWGHKAIAITDHANVQSFPHGYHRARKAGIKAIFGLEANIVEDKVPISYEPVDMDLHEATYVVFDVETTGLSAMNNDLIQIAASKMFKGNIVEQFDEFIDPGHPLSAFTTELTGITDKHLQGAKPLVTVLKAFQDFCKDSILVAHNASFDVGFMNANYERHDLPKITQPVIDTLEFARNLYPEYKRHGLGPLTKRFQVSLDHHHMANYDAEATGRLLFIFLKDAREKHGIKNLLQLNTDLVAEDSYKKARIKHATIYVQNQVGLKNMFKLVSLSNIKYFEGVPRIPRTVLDAHREGLLLGTACSDGEVFDAVLTKGIDAAVDLARYYDFIEIMPPAIYQPLVVRELIKDQAGIEQVIRDLIEVGKRAKKPVLATGNVHYLEPEEEIYREIIVRSLGQGAMINRTIGRGEGAQPAPLPKAHFRTTNEMLDEFAFLGKDLAYQVVVQNTQDFADRIEEVEVVKGDLYTPYIDKAEETVAELTYQKAFEIYGNPLPDIIDLRIEKELTSILGNGFAVIYLASQMLVNRSNERGYLVGSRGSVGSSFVATMIGITEVNPMPPHYVCPSCQHSEFITDGSVGSGYDLPNKPCPKCGTPYQKDGQDIPFETFLGFDGDKVPDIDLNFSGDDQPSAHLDVRDIFGDEYAFRAGTVGTVAEKTAYGFVKGYERDYGKFYRDAEVDRLAAGAAGVKRTTGQHPGGIVVIPNYMDVYDFTPVQYPADDVTASWQTTHFNFHDIDENVLKLDILGHDDPTMIRKLQDLSGIDPITIPADDPGVMALFSGTEVLGVTPEQIGTPTGMLGIPEFGTNFVRGMVNETHPTTFAELLQLSGLSHGTDVWLGNAQDLIKEGIATLKTVIGCRDDIMVYLMHAGLEPKMAFTIMERVRKGLWLKISEEERNGYIDAMRENNVPDWYIESCGKIKYMFPKAHAAAYVLMALRVAYFKVHHPIMYYCAYFSIRAKAFELKTMSGGLDAVKARMEDITIKRKNNEATNVENDLFTTLEIVNEMLERGFKFGKLDLYKSDAIEFQIKGDTLIPPFIALEGLGENVAKQIVKARQEGEFLSKMELRKRGGASSTLVEKMDEMGILGNMPEDNQLSLFDDFF

SEQ ID NO: 84 - DNA, 4398 bp, Streptococcus pyogenes
atgtcagatttattcgctaaattgatggaccagatagaaatgccacttgacatgagacgttcaagtgccttttcatctgctgatattatcgaggtaaaggtacattcggtgtcacgcttgtgggaatttcattttgcctttgcagcggttttaccgattgcaacttatcgtgaattgcatgatcgtttgataagaacttttgaggcggctgacattaaggtaacctttgacatccaagctgctcaggtggattattcagatgatctgcttcaagcttattaccaagaagcttttgagcatgcaccgtgtaatagtgctagttttaaatcttctttctcaaagctcaaagtgacttatgaggatgacaaactcattattgcagcgccaggttttgtgaataacgatcattttagaaacaatcatctgcctaatctggtcaagcaattagaagcctttggctttggcatcttgaccatagatatggtgtcagatcaggaaatgactgagcatttgaccaagaattttgtttccagtcgtcaggctcttgtgaaaaaggctgtgcaggataatttggaagcccaaaaatctcttgaagccatgatgccaccagttgaggaagccacacctgctcctaagtttgactacaaggaacgagcagctaagcgtcaggcagggtttgaaaaagcaaccatcacaccaatgattgagattgagaccgaagaaaaccggattgtctttgagggtatggtttttgacgtggagcgtaaaacgactaggacaggtcgccatatcatcaactttaaaatgacagactatacctcctcgtttgctctccaaaaatgggctaaagacgatgaggagctccgtaaatttgatatgattgctaagggagcttggttacgggtacaagggaatattgagaccaatccttttacgaagagtctcaccatgaatgtccagcaggtcaaagaaattgtccgtcatgagcgcaaagacctgatgccagaagggcaaaagcgggtcgaacttcatgcccacaccaatatgtctaccatggatgccttaccgacagtagaaagcttgattgatacggcagccaagtggggacacaaggcgattgctatcaccgaccatgctaatgtgcaaagttttcctcatggctaccatagggctcgcaaagctgggattaaggctatttttggcctagaagccaatattgttgaggacaaggtgcctatttcttatgaacctgttgatatggatttgcacgaagctacctatgtggtctttgacgtggaaaccacaggtctatctgctatgaataatgacctgattcagattgcggcttccaaaatgtttaaaggaaatattgtagagcagtttgatgaattcattgatcctgggcatcctctttcagcctttaccaccgaattgacaggaattaccgataagcatttgcagggcgccaagccattggttactgtcctaaaagcttttcaggacttttgcaaagatagtatcttggttgcccacaacgccagttttgacgtgggctttatgaacgccaattatgaacgccacgacttgcccaaaatcacacagcctgtgattgataccttagaatttgctagaaacttgtatcctgagtacaagcgtcacggtttgggaccgctcaccaagcgtttccaagtgagtctagaccaccatcatatggccaattacgacgcggaagccacaggacgtcttttgtttatttttctaaaagatgccagagaaaagcatggcatcaaaaatcttttgcaactcaatacagatttggtggctgaggattcttacaaaaaagcgcggattaagcatgcgactatctatgtgcaaaatcaggttggtcttaaaaatatgtttaagttggtcagcctttccaatatcaaatattttgaaggggtgccgcgtattccaagaaccgtcttagatgctcacagagagggtttgttacttggtacagcttgttctgacggcgaggtttttgatgccgttctgactaaaggaattgatgcagcggttgatttggctaggtattatgattttatcgaaatcatgccaccagccatttaccagccattggttgtccgtgaattaatcaaagatcaagcaggtattgagcaggtgattcgtgacctcattgaagtagggaaacgagctaagaaacctgtgcttgccactgggaatgtgcattatctagagcctgaagaagagatttaccgtgaaattattgtgcgtagtcttggtcagggtgccatgattaatagaacaatcggccgtggggaaggggcacagcctgctcctctacctaaagcgcactttagaacaaccaatgaaatgctggatgagtttgcctttcttggaaaagacctcgcttatcaagtagttgtgcaaaatactcaggattttgcggaccgtattgaggaagtggaagtggttaagggcgatctttacaccccgtatattgataaggccgaagagacggttgccgaattaacctatcaaaaagcctttgaaatttatggtaatcctctcccagatattattgatttacgcattgaaaaagagttaacctctatcttggggaacggttttgctgtgatttatctcgcttcccaaatgcttgttaaccggtcaaatgagcgaggctacctagttggttctaggggatctgtagggtctagctttgtcgccaccatgattgggattactgaggttaatcctatgccgcctcactacgtttgcccgtcctgccaacattctgaatttatcacagatgggtcagttggatctggctatgatttgcctaataaaccctgtccgaaatgtggcaccccttatcaaaaagatgggcaagacattccctttgagacctttcttgggtttgatggggataaggtgcccgatattgatttgaacttctctggtgatgaccagcccagtgcccatttggatgtccgagatatttttggtgacgaatacgcctttcgtgctggaacagttggtaccgtagcagaaaaaacagcttatggatttgtcaaaggctatgaacgcgactatggcaagttctatcgtgatgctgaggtggatcgtctagcagcaggtgctgctggtgtgaaacgaacgactgggcagcaccctggggggattgttgttattcctaattacatggatgtttatgattttacccccgtgcaatatccagccgatgatgtaacggcttcttggcagacaactcactttaacttccatgatattgatgaaaacgtcttgaaacttgatatcctagggcatgatgatccgaccatgattcgtaaacttcaggatttatcgggcattgatcctattactattcctgctgatgatccgggagttatggctctcttttctgggacagaggttttgggcgttaccccggaacaaattgggacaccgactggtatgctaggcattccagaatttggaaccaactttgttcgcggcatggttaatgagacgcatccgaccacttttgcggagcttttgcagttgtctggactatctcatggaaccgatgtttggcttggtaatgcacaagatttgattaaagaaggcattgcaaccctaaaaaccgttatcggttgtcgtgacgacatcatggtttacctcatgcacgcaggcttagaaccaaaaatggcctttaccattatggagcgtgtgcgtaagggattatggctaaaaatttctgaggaagaacgtaatggctatattgatgccatgcgagaaaacaatgtgcccgactggtacattgaatcgtgtggaaaaatcaagtacatgttccctaaagcccatgcggcagcttatgttttgatggcccttcgggtggcttatttcaaggtgcaccaccccattatgtattattgtgcttatttctctattcgtgcgaaggcttttgaattaaaaaccatgagtggtggtttagatgctgttaaagcaagaatggaagatattactataaaacgtaaaaataatgaagccaccaatgtggaaaatgacctctttacaaccttggagattgtcaacgaaatgttagaacgcggctttaagtttggcaaattagacctttacaaaagtgatgctatagaattccaaatcaaaggagatacccttatccctccatttatagcgctagaaggtctgggtgaaaacgtggccaagcaaatcgttaaagctcgtcaagaaggcgaattcctctctaaaatggaattgcgtaaacgaggcggggcatcgtcaacgctcgttgagaaaatggatgagatgggtattttaggaaatatgccagaagataatcaattaagtctttttgatgactttttctag

SEQ ID NO: 85 - DNA, 4398 bp, Streptococcus pyogenes
ctagaaaaagtcatcaaaaagacttaattgattatcttctggcatatttcctaaaatacccatctcatccattttctcaacgagcgttgacgatgccccgcctcgtttacgcaattccattttagagaggaattcgccttcttgacgagctttaacgatttgcttggccacgttttcacccagaccttctagcgctataaatggagggataagggtatctcctttgatttggaattctatagcatcacttttgtaaaggtctaatttgccaaacttaaagccgcgttctaacatttcgttgacaatctccaaggttgtaaagaggtcattttccacattggtggcttcattatttttacgttttatagtaatatcttccattcttgctttaacagcatctaaaccaccactcatggtttttaattcaaaagccttcgcacgaatagagaaataagcacaataatacataatggggtggtgcaccttgaaataagccacccgaagggccatcaaaacataagctgccgcatgggctttagggaacatgtacttgatttttccacacgattcaatgtaccagtcgggcacattgttttctcgcatggcatcaatatagccattacgttcttcctcagaaatttttagccataatcccttacgcacacgctccataatggtaaaggccatttttggttctaagcctgcgtgcatgaggtaaaccatgatgtcgtcacgacaaccgataacggtttttagggttgcaatgccttctttaatcaaatcttgtgcattaccaagccaaacatcggttccatgagatagtccagacaactgcaaaagctccgcaaaagtggtcggatgcgtctcattaaccatgccgcgaacaaagttggttccaaattctggaatgcctagcataccagtcggtgtcccaatttgttccggggtaacgcccaaaacctctgtcccagaaaagagagccataactcccggatcatcagcaggaatagtaataggatcaatgcccgataaatcctgaagtttacgaatcatggtcggatcatcatgccctaggatatcaagtttcaagacgttttcatcaatatcatggaagttaaagtgagttgtctgccaagaagccgttacatcatcggctggatattgcacgggggtaaaatcataaacatccatgtaattaggaataacaacaatccccccagggtgctgcccagtcgttcgtttcacaccagcagcacctgctgctagacgatccacctcagcatcacgatagaacttgccatagtcgcgttcatagcctttgacaaatccataagctgttttttctgctacggtaccaactgttccagcacgaaaggcgtattcgtcaccaaaaatatctcggacatccaaatgggcactgggctggtcatcaccagagaagttcaaatcaatatcgggcaccttatccccatcaaacccaagaaaggtctcaaagggaatgtcttgcccatctttttgataaggggtgccacatttcggacagggtttattaggcaaatcatagccagatccaactgacccatctgtgataaattcagaatgttggcaggacgggcaaacgtagtgaggcggcataggattaacctcagtaatcccaatcatggtggcgacaaagctagaccctacagatcccctagaaccaactaggtagcctcgctcatttgaccggttaacaagcatttgggaagcgagataaatcacagcaaaaccgttccccaagatagaggttaactctttttcaatgcgtaaatcaataatatctgggagaggattaccataaatttcaaaggctttttgataggttaattcggcaaccgtctcttcggccttatcaatatacggggtgtaaagatcgcccttaaccacttccacttcctcaatacggtccgcaaaatcctgagtattttgcacaactacttgataagcgaggtcttttccaagaaaggcaaactcatccagcatttcattggttgttctaaagtgcgctttaggtagaggagcaggctgtgccccttccccacggccgattgttctattaatcatggcaccctgaccaagactacgcacaataatttcacggtaaatctcttcttcaggctctagataatgcacattcccagtggcaagcacaggtttcttagctcgtttccctacttcaatgaggtcacgaatcacctgctcaatacctgcttgatctttgattaattcacggacaaccaatggctggtaaatggctggtggcatgatttcgataaaatcataatacctagccaaatcaaccgctgcatcaattcctttagtcagaacggcatcaaaaacctcgccgtcagaacaagctgtaccaagtaacaaaccctctctgtgagcatctaagacggttcttggaatacgcggcaccccttcaaaatatttgatattggaaaggctgaccaacttaaacatatttttaagaccaacctgattttgcacatagatagtcgcatgcttaatccgcgcttttttgtaagaatcctcagccaccaaatctgtattgagttgcaaaagatttttgatgccatgcttttctctggcatcttttagaaaaataaacaaaagacgtcctgtggcttccgcgtcgtaattggccatatgatggtggtctagactcacttggaaacgcttggtgagcggtcccaaaccgtgacgcttgtactcaggatacaagtttctagcaaattctaaggtatcaatcacaggctgtgtgattttgggcaagtcgtggcgttcataattggcgttcataaagcccacgtcaaaactggcgttgtgggcaaccaagatactatctttgcaaaagtcctgaaaagcttttaggacagtaaccaatggcttggcgccctgcaaatgcttatcggtaattcctgtcaattcggtggtaaaggctgaaagaggatgcccaggatcaatgaattcatcaaactgctctacaatatttcctttaaacattttggaagccgcaatctgaatcaggtcattattcatagcagatagacctgtggtttccacgtcaaagaccacataggtagcttcgtgcaaatccatatcaacaggttcataagaaataggcaccttgtcctcaacaatattggcttctaggccaaaaatagccttaatcccagctttgcgagccctatggtagccatgaggaaaactttgcacattagcatggtcggtgatagcaatcgccttgtgtccccacttggctgccgtatcaatcaagctttctactgtcggtaaggcatccatggtagacatattggtgtgggcatgaagttcgacccgcttttgcccttctggcatcaggtctttgcgctcatgacggacaatttctttgacctgctggacattcatggtgagactcttcgtaaaaggattggtctcaatattcccttgtacccgtaaccaagctcccttagcaatcatatcaaatttacggagctcctcatcgtctttagcccatttttggagagcaaacgaggaggtatagtctgtcattttaaagttgatgatatggcgacctgtcctagtcgttttacgctccacgtcaaaaaccataccctcaaagacaatccggttttcttcggtctcaatctcaatcattggtgtgatggttgctttttcaaaccctgcctgacgcttagctgctcgttccttgtagtcaaacttaggagcaggtgtggcttcctcaactggtggcatcatggcttcaagagatttttgggcttccaaattatcctgcacagcctttttcacaagagcctgacgactggaaacaaaattcttggtcaaatgctcagtcatttcctgatctgacaccatatctatggtcaagatgccaaagccaaaggcttctaattgcttgaccagattaggcagatgattgtttctaaaatgatcgttattcacaaaacctggcgctgcaataatgagtttgtcatcctcataagtcactttgagctttgagaaagaagatttaaaactagcactattacacggtgcatgctcaaaagcttcttggtaataagcttgaagcagatcatctgaataatccacctgagcagcttggatgtcaaaggttaccttaatgtcagccgcctcaaaagttcttatcaaacgatcatgcaattcacgataagttgcaatcggtaaaaccgctgcaaaggcaaaatgaaattcccacaagcgtgacaccgaatgtacctttacctcgataatatcagcagatgaaaaggcacttgaacgtctcatgtcaagtggcatttctatctggtccatcaatttagcgaataaatctgacat